SEQUENCE LISTING (15 sequences)

SEQ ID NO: 1 - protein, 23 aa, ARTIFICIAL SEQUENCE
  MCL-1 binding domain
RPEIWMTQGLRRLGDEINAYYAR

SEQ ID NO: 2 - protein, 23 aa, ARTIFICIAL SEQUENCE
  MCL-1 binding domain
RPEIWLTQSLQRLGDEINAYYAR

SEQ ID NO: 3 - protein, 23 aa, ARTIFICIAL SEQUENCE
  MCL-1 binding domain
RPEIWLTQHLQRLGDEINAYYAR

SEQ ID NO: 4 - protein, 23 aa, ARTIFICIAL SEQUENCE
  MCL-1 binding domain
RPEIWMGQGLRRLGDEINAYYAR

SEQ ID NO: 5 - protein, 23 aa, ARTIFICIAL SEQUENCE
  MCL-1 binding domain
RPEIWLGQSLQRLGDEINAYYAR

SEQ ID NO: 6 - protein, 23 aa, ARTIFICIAL SEQUENCE
  MCL-1 binding domain
RPEIWLGQHLQRLGDEINAYYAR

SEQ ID NO: 7 - protein, 23 aa, ARTIFICIAL SEQUENCE
  MCL-1 binding domain
RPEIWITQELRRIGDEFNAYYAR

SEQ ID NO: 8 - protein, 23 aa, ARTIFICIAL SEQUENCE
  MCL-1 binding domain
RPEIWMTQELRRIGDEFNAYYAR

SEQ ID NO: 9 - protein, 23 aa, ARTIFICIAL SEQUENCE
  MCL-1 binding domain
RPEIWITQGLRRIGDEFNAYYAR

SEQ ID NO: 10 - protein, 23 aa, ARTIFICIAL SEQUENCE
  MCL-1 binding domain
RPEIWITQELRRLGDEFNAYYAR

SEQ ID NO: 11 - protein, 23 aa, ARTIFICIAL SEQUENCE
  MCL-1 binding domain
RPEIWITQELRRIGDEINAYYAR

SEQ ID NO: 12 - protein, 11 aa, ARTIFICIAL SEQUENCE
  TAT translocation domain
YGRKKRRQRRR

SEQ ID NO: 13 - protein, 16 aa, ARTIFICIAL SEQUENCE
  ANT translocation domain
RQIKIWFQNRRMKWKK

SEQ ID NO: 14 - protein, 37 aa, ARTIFICIAL SEQUENCE
  MCL-1 profiling peptide
YGRKKRRQRRRGGGRPEIWMTQGLRRLGDEINAYYAR

SEQ ID NO: 15 - protein, 37 aa, ARTIFICIAL SEQUENCE
  MCL-1 profiling peptide
RPEIWMTQGLRRLGDEINAYYARGGGYGRKKRRQRRR

(19) United States
(12) Patent Application Publication
(10) Pub. No.: US 2021/0277037 A1
(43) Pub. Date: Sep. 9, 2021

(54) DEUTERATED ALVOCIDIB AND ALVOCIDIB PRODRUGS

(71) Applicant: SUMITOMO DAINIPPON PHARMA ONCOLOGY, INC., Cambridge, MA (US)

(72) Inventor: Adam SIDDIQUI-JAIN, South Jordan, UT (US)

(21) Appl. No.: 17/253,603

(22) PCT Filed: Jun. 20, 2019
(86) PCT No.: PCT/US2019/038283

§ 371 Date: Dec. 17, 2020

Related U.S. Application Data
(60) Provisional application No. 62/688,140, filed on Jun. 21, 2018.

(51) Int. Cl. C07F 9/141; C07D 405/04; A61K 45/06; A61K 31/706

(52) U.S. Cl. CPC C07F 9/1411; C07D 405/04; C07B 2200/07; A61K 31/706; A61K 45/06

(57) ABSTRACT

Provided herein are compounds having the following structure (I) or a stereoisomer, tautomer or pharmaceutically acceptable salt thereof, wherein R¹, R², R³, R⁴, R⁵, R^{a}, R^{b}, and R^{c} are as defined herein. Also provided are pharmaceutical compositions comprising compounds of structure (I), or a stereoisomer, tautomer or pharmaceutically acceptable salt thereof, and methods for use of compounds of structure (I), or a stereoisomer, tautomer or pharmaceutically acceptable salt thereof, for treating diseases associated with overexpression of a cyclin-dependent kinase (CDK).

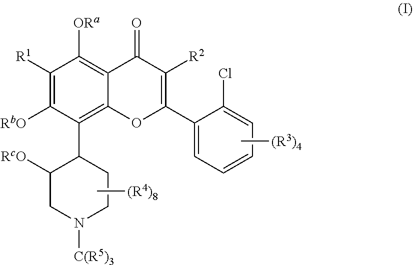

### RELATED APPLICATIONS

This application claims the benefit of U.S. Provisional Application No. 62/688,140, filed on Jun. 21, 2018. The entire teachings of this application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 56361002001_Sequence_Listing.txt; created Jun. 5, 2019, 9 KB in size.

### BACKGROUND

Cyclin-dependent kinases (CDKs) are important regulators that control the timing and coordination of the cell cycle. CDKs form reversible complexes with their obligate cyclin partners to control transition through key junctures in the cell cycle. For example, the activated CDK4-cyclin D1 complex controls progression through the G1 phase of the cell cycle, while the CDK1-cyclin B1 complex controls entry into the mitotic phase of the cell cycle. Endogenous CDK inhibitory proteins (CDKIs) are known to bind either the CDK or cyclin component and inhibit the kinase activity of the complex. In many tumors, such as melanomas, and pancreatic and esophageal cancers, these natural CDKIs are either absent or mutated.

Alvocidib, also known as flavopiridol, is a potent and selective inhibitor of the CDKs, has antitumor activity against various tumor cells lines, such as human lung carcinoma and breast carcinoma, and also inhibits tumor growth in xenograft models. Alvocidib has been shown to induce arrest in both the G1 and G2 phases of the cell cycle and also inhibit polymerase II driven transcription by inhibiting CDK9. By inhibiting CDK9, which forms part of the complex known as the positive transcription elongation factor or P-TEFb, alvocidib reduces the expression of key oncogenes such MYC and key anti-apoptotic proteins such as MCL1. Accordingly, alvocidib is an attractive therapeutic agent for cancer and is currently undergoing clinical trials in relapsed/refractory AML patients.

Oral administration of alvocidib has been limited by gastrointestinal toxicity and limited oral bioavailability. Further, preclinical studies suggest that prolonged exposure may be important for maximizing alvocidib's activity, prompting extensive exploration of continuous intravenous infusion of alvocidib in human trials. Alternative hybrid dosing, including an intravenous bolus dose followed by a slow infusion, has also been explored.

Nonetheless, there remains a need for new forms of alvocidib that increase oral bioavailability of alvocidib and/or require less frequent and/or prolonged dosing for efficacy.

### SUMMARY

In one embodiment is provided a compound having the following structure (I):

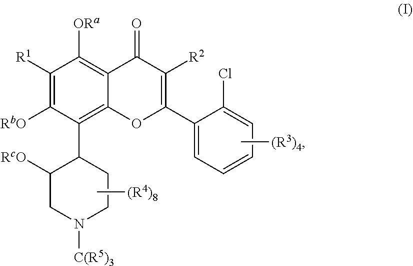

or a stereoisomer, tautomer or pharmaceutically acceptable salt thereof, wherein:
R^{a}, R^{b} and R^{c} are each independently H, D or —P(═O)(OR)₂; and
R, R¹, R², R³, R⁴ and R⁵ are, at each occurrence, independently H or D,
wherein at least one occurrence of R, R^{a}, R^{b}, R^{c}, R¹, R², R³, R⁴ and R⁵ is D.

Other embodiments are directed to a pharmaceutical composition comprising a pharmaceutically acceptable carrier or excipient and a compound of structure (I), or a stereoisomer, tautomer or pharmaceutically acceptable salt thereof. Methods for use of the compound of structure (I), or a stereoisomer, tautomer or pharmaceutically acceptable salt thereof, and pharmaceutical compositions comprising the same, for treatment of a disease associated with overexpression of a cyclin-dependent kinase (CDK) in a mammal in need thereof are also provided.

These and other aspects of the disclosure will be apparent upon reference to the following detailed description. To this end, various references are set forth herein which describe in more detail certain background information, procedures, compounds and/or compositions, and are each hereby incorporated by reference in their entirety.

### DETAILED DESCRIPTION

A description of example embodiments follows.

In the following description, certain specific details are set forth in order to provide a thorough understanding of various embodiments of the disclosure. However, one skilled in the art will understand that these embodiments may be practiced without these details.

It will be recognized that some variation of natural isotopic abundance occurs in a synthesized compound depending upon the origin of chemical materials used in the synthesis. Thus, preparation of alvocidib and prodrugs thereof (e.g., phosphate prodrugs) will inherently contain small amounts of deuterated isotopologues. The concentration of naturally abundant stable hydrogen and carbon isotopes, notwithstanding this variation, is small and immaterial as compared to the degree of stable isotopic substitution of compounds of this disclosure.

Unless the context requires otherwise, throughout the present specification and claims, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Embodiments of the present disclosure include phosphate prodrugs of alvocidib. “Phosphate” refers to a —OP(═O)(OR)₂ moiety, wherein R is, at each occurrence, independently H or D. For ease of illustration, the phosphate moieties herein are often depicted in the di-protonated/di-deuterated form, but also exist in the mono-protonated (—OP(═O)(OH)(O⁻)) or mono-deuterated (—OP(═O)(OD)(O⁻)) and unprotonated forms (—OP(═O)(O⁻)₂), depending on pH. The mono-protonated, mono-deuterated and unprotonated forms will typically be associated with a counterion, such that the compounds are in the form of a pharmaceutically acceptable salt. Such mono-protonated, mono-deuterated and unprotonated forms, and their pharmaceutically acceptable salts, are encompassed within the scope of the disclosure, even if not specifically illustrated in a chemical structure.

“Prodrug” is meant to indicate a compound that may be converted under physiological conditions or by solvolysis to a biologically active compound described herein (e.g., compound of structure (I) wherein R^{a}, R^{b} and R^{c} are each independently H or D, or a monoprotonated, mono-deuterated or unprotonated form thereof, or a pharmaceutically acceptable salt thereof). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

A “compound of the disclosure” refers to a compound of structure (I), and its substructures, as defined herein, as well as its stereoisomers, tautomers and prodrugs, and salts, particularly pharmaceutically acceptable salts, of any of the foregoing. As used herein, “compound” refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. The relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

Embodiments of the disclosure as disclosed herein are also meant to encompass all pharmaceutically acceptable compounds of structure (I) being isotopically-labelled by having one or more non-deuterium atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radio-labelled compounds could be useful to help determine or measure the effectiveness of the compounds, by characterizing, for example, the site or mode of action, or binding affinity to a pharmacologically important site of action. Certain isotopically-labelled compounds of structure (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H or D, may afford certain therapeutic advantages resulting from greater metabolic stability, for example, increased in vivo half-life or reduced dosage requirements and, hence, may be preferred in some circumstances.

Substitution with positron emitting isotopes, such as ¹¹C, ¹⁸F, ¹⁵O and ¹³N, can be useful in positron emission topography (PET) studies for examining substrate receptor occupancy. Isotopically-labeled compounds of structure (I) can generally be prepared by conventional techniques known to those skilled in the art or by processes analogous to those described in the Preparations and Examples, as set out below using an appropriate isotopically-labeled reagent in place of the non-labeled reagent previously employed.

Molecules or compounds that differ only by their isotopic composition, such as in the foregoing examples, are referred to herein as “isotopologues.” An “isotopologue” refers to a chemical compound or chemical species that has at least one atom with a different number of neutrons than the parent (e.g., at least one ¹H of the parent is replaced by ²H, or D). Accordingly, one embodiment provides an isotopologue of alvocidib.

“Isotopic enrichment factor” refers to the ratio between the isotopic abundance and the natural abundance of a specified isotope.

Embodiments disclosed herein are also meant to encompass the in vivo metabolic products of the compounds of the disclosure. Such products may result from, for example, the oxidation, reduction, hydrolysis, amidation, esterification, and the like, of the administered compound, primarily due to enzymatic processes. Accordingly, embodiments of the disclosure include compounds produced by a process comprising administering a compound of the disclosure to a mammal for a period of time sufficient to yield a metabolic product thereof. Such products are typically identified by administering a radiolabelled compound of the disclosure in a detectable dose to an animal, such as a rat, mouse, guinea pig, monkey, or human, allowing sufficient time for metabolism to occur, and isolating its conversion products from the urine, blood or other biological samples.

“Pharmaceutically acceptable carrier, diluent or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

As used herein, the term “pharmaceutically acceptable salt” refers to those salts that are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. “Pharmaceutically acceptable salt” includes both acid and base addition salts.

“Pharmaceutically acceptable acid addition salt” refers to a pharmaceutically acceptable salt formed with an acid that is not biologically or otherwise undesirable. In some embodiments, a pharmaceutically acceptable acid addition salt retains the biological effectiveness and properties of its corresponding free base, is not biologically or otherwise undesirable, and is formed with inorganic acids such as, but not limited to, hydrochloric acid, hydrobromic acid, sulfuric acid, nitric acid, phosphoric acid and the like, and/or organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to a pharmaceutically acceptable salt formed with a base that is not biologically or otherwise undesirable. In some embodiments, a pharmaceutically acceptable base addition salt retains the biological effectiveness and properties of its corresponding free acid. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts, and the like. Preferred inorganic salts are the ammonium, sodium, potassium, calcium, and magnesium salts. Salts derived from organic bases include, but are not limited to, salts of primary, secondary, and tertiary amines, substituted amines including naturally-occurring substituted amines, cyclic amines and basic ion-exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Preferred organic bases are isopropylamine, diethylamine, ethanolamine, trimethylamine, dicyclohexylamine, choline and caffeine.

Often crystallizations produce a solvate of the compound of the disclosure. As used herein, the term “solvate” refers to an aggregate that comprises one or more molecules of a compound of the disclosure with one or more molecules of solvent. The solvent may be water, in which case the solvate is a hydrate. Alternatively, the solvent may be an organic solvent. Thus, compounds of the present disclosure may exist as a hydrate, including a monohydrate, dihydrate, hemihydrate, sesquihydrate, trihydrate, tetrahydrate and the like, as well as the corresponding solvated forms. In some cases, the compound of the disclosure may be a true solvate, while in other cases, the compound of the disclosure may merely retain adventitious water or be a mixture of water plus some adventitious solvent.

A “pharmaceutical composition” refers to a formulation of a compound of the disclosure and a medium generally accepted in the art for the delivery of a biologically active compound to mammals, e.g., humans. Such a medium includes all pharmaceutically acceptable carriers, diluents or excipients therefor.

“Mammal” includes humans and both domestic animals such as laboratory animals and household pets (e.g., cats, dogs, swine, cattle, sheep, goats, horses, rabbits), and non-domestic animals, such as wildlife and the like.

“Effective amount” and “therapeutically effective amount” are used interchangeably herein, and each refers to that amount of a compound of the disclosure which, when administered to a mammal, preferably a human, is sufficient to effect treatment, as defined below, of a disease associated with overexpression of a cyclin-dependent kinase (CDK) in the mammal, preferably a human. The amount of a compound of the disclosure which constitutes a “therapeutically effective amount” will vary depending on, inter alia, the compound, the condition and its severity, the manner of administration, and characteristics (e.g., the age) of the mammal to be treated, but can be determined routinely by one of ordinary skill in the art having regard to his own knowledge and to this disclosure.

“Treating” or “treatment,” as used herein, refers to the administration of a medication or medical care to a subject, such as a human, having a disease or condition of interest, e.g., a cancer, and includes: (i) preventing the disease or condition from occurring in a subject, in particular, when such subject is predisposed to the condition but has not yet been diagnosed as having it; (ii) inhibiting the disease or condition, e.g., arresting its development; (iii) relieving the disease or condition, e.g., causing regression of the disease or condition; or (iv) relieving the symptoms resulting from the disease or condition, (e.g., pain, weight loss, cough, fatigue, weakness, etc.).

As used herein, the terms “disease” and “condition” may be used interchangeably or may be different in that the particular malady or condition may not have a known causative agent (so that etiology has not yet been confirmed) and it is therefore not yet recognized as a disease but only as an undesirable condition or syndrome, wherein a more or less specific set of symptoms has been identified by clinicians.

The compounds of the disclosure may contain one or more centers or bonds that result in the compound having an overall asymmetry and may thus give rise to enantiomers, diastereomers, and other stereoisomeric forms that may be defined, in terms of absolute stereochemistry, as (R)- or (S)-, or as (D)- or (L)- for amino acids. Unless otherwise specified, the present disclosure is meant to include all such possible isomers, as well as their racemic and optically pure forms, and mixtures thereof. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds of the disclosure contain olefinic double bonds or other center giving rise to geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. Embodiments of the present disclosure contemplate various stereoisomers and mixtures thereof. These embodiments include “enantiomers,” which refers to two stereoisomers whose molecules are non-superimposable mirror images of one another.

A “tautomer” refers to a proton shift from one atom of a molecule to another atom of the same molecule. Embodiments of the present disclosure include tautomers of the compounds of the disclosure.

### Compounds

One embodiment provides a compound having the following structure (I):

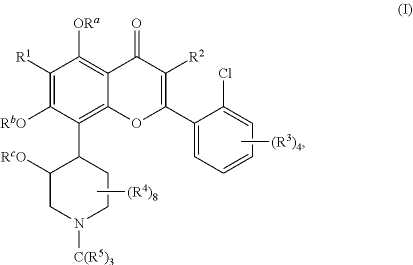

or a stereoisomer, tautomer or pharmaceutically acceptable salt thereof, wherein:

R^{a}, R^{b} and R^{c} are each independently H, D or —P(═O)(OR)₂;

R, R¹, R², R³, R⁴ and R⁵ are, at each occurrence, independently H or D; wherein at least one occurrence of R, R^{a}, R^{b}, R^{c}, R¹, R², R³, R⁴ and R⁵ is D.

In some embodiments, the compound has the following structure (I′):

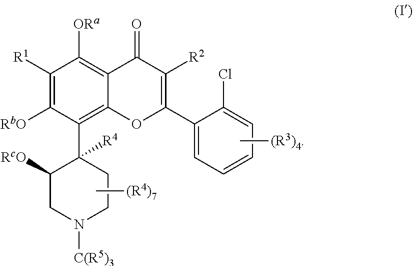

In certain more specific embodiments, the compound has the following structure (I″):

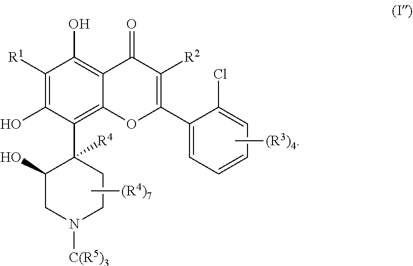

In certain other specific embodiments, the compound has the following structure (IA):

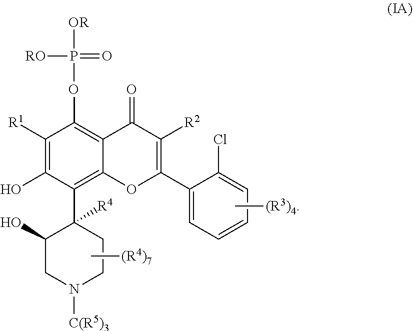

In some embodiments, the compound has the following structure (IB):

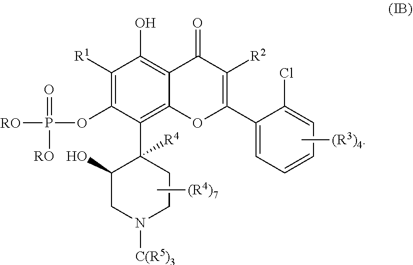

In certain embodiments, the compound has the following structure (IC):

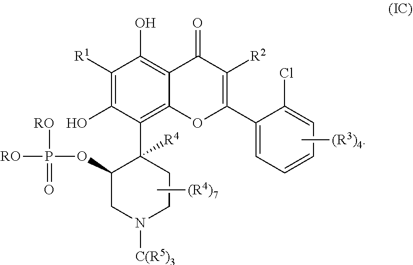

In some of the foregoing embodiments, R¹ is D. In some of the foregoing embodiments, R² is D.

In some of the foregoing embodiments, R¹ is H. In some of the foregoing embodiments, R² is H.

In some embodiments, at least one occurrence of R³ is D. In some related embodiments, R³ is D at each occurrence.

In some embodiments, at least one occurrence of R³ is H. In some related embodiments, R³ is H at each occurrence.

In certain embodiments, at least one occurrence of R⁴ is D. In more specific embodiments, R⁴ is D at each occurrence.

In certain embodiments, at least one occurrence of R⁴ is H. In more specific embodiments, R⁴ is H at each occurrence.

In some embodiments, at least one occurrence of R⁵ is D. In certain more specific embodiments, R⁵ is D at each occurrence.

In some embodiments, at least one occurrence of R⁵ is H. In certain more specific embodiments, R⁵ is H at each occurrence.

In some embodiments, at least one occurrence of R is D. In more specific embodiments, R is D at each occurrence. In some embodiments, at least one occurrence of R is H. In more specific embodiments, R is H at each occurrence.

In some embodiments, the compound is selected from Table 1 below.

TABLE 1
Exemplary compounds of Structure (I)

| No. | Structure |
|---|---|
| I-1 | 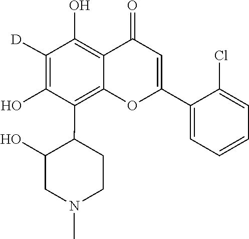 [SMILES: [2H]c1c(O)c(C2CCN(C)CC2O)c2oc(-c3ccccc3Cl)cc(=O)c2c1O] |
| I″-1 | 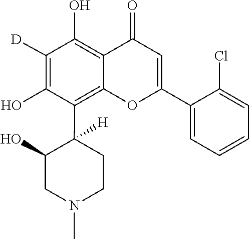 [SMILES: [2H]c1c(O)c([C@H]2CCN(C)C[C@H]2O)c2oc(-c3ccccc3Cl)cc(=O)c2c1O] |
| I-2 | 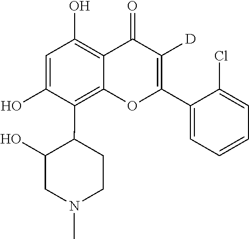 [SMILES: [2H]c1c(-c2ccccc2Cl)oc2c(C3CCN(C)CC3O)c(O)cc(O)c2c1=O] |
| I″-2 | 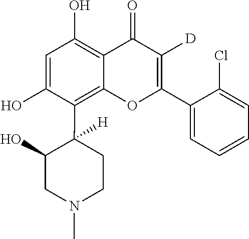 [SMILES: [2H]c1c(-c2ccccc2Cl)oc2c([C@H]3CCN(C)C[C@H]3O)c(O)cc(O)c2c1=O] |
| I-3 | 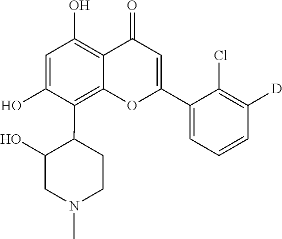 [SMILES: [2H]c1cccc(-c2cc(=O)c3c(O)cc(O)c(C4CCN(C)CC4O)c3o2)c1Cl] |
| I″-3 | 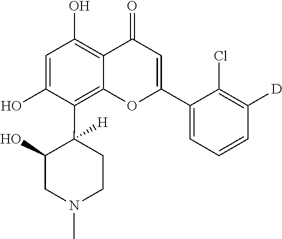 [SMILES: [2H]c1cccc(-c2cc(=O)c3c(O)cc(O)c([C@H]4CCN(C)C[C@H]4O)c3o2)c1Cl] |
| I-4 | 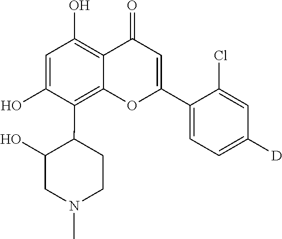 [SMILES: [2H]c1ccc(-c2cc(=O)c3c(O)cc(O)c(C4CCN(C)CC4O)c3o2)c(Cl)c1] |
| I″-4 | 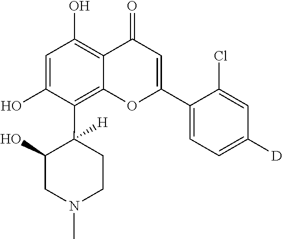 [SMILES: [2H]c1ccc(-c2cc(=O)c3c(O)cc(O)c([C@H]4CCN(C)C[C@H]4O)c3o2)c(Cl)c1] |
| I-5 | 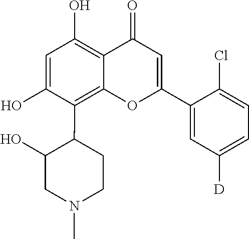 [SMILES: [2H]c1ccc(Cl)c(-c2cc(=O)c3c(O)cc(O)c(C4CCN(C)CC4O)c3o2)c1] |
| I″-5 | 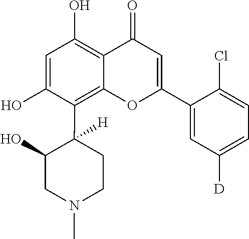 [SMILES: [2H]c1ccc(Cl)c(-c2cc(=O)c3c(O)cc(O)c([C@H]4CCN(C)C[C@H]4O)c3o2)c1] |
| I-6 | 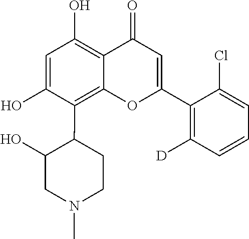 [SMILES: [2H]c1cccc(Cl)c1-c1cc(=O)c2c(O)cc(O)c(C3CCN(C)CC3O)c2o1] |
| I″-6 | 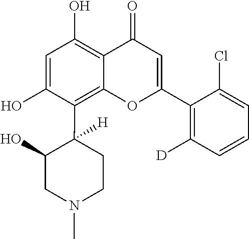 [SMILES: [2H]c1cccc(Cl)c1-c1cc(=O)c2c(O)cc(O)c([C@H]3CCN(C)C[C@H]3O)c2o1] |
| I-7 | 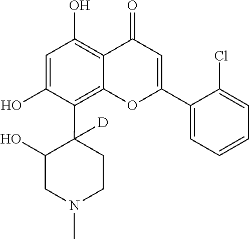 [SMILES: [2H]C1(c2c(O)cc(O)c3c(=O)cc(-c4ccccc4Cl)oc23)CCN(C)CC1O] |
| I″-7 | 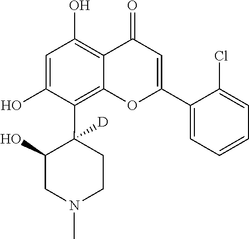 [SMILES: [2H][C@]1(c2c(O)cc(O)c3c(=O)cc(-c4ccccc4Cl)oc23)CCN(C)C[C@H]1O] |
| I-8 | 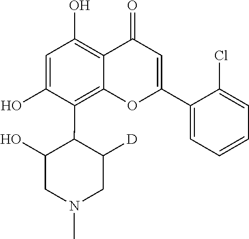 [SMILES: [2H]C1CN(C)CC(O)C1c1c(O)cc(O)c2c(=O)cc(-c3ccccc3Cl)oc12] |
| I″-8 | 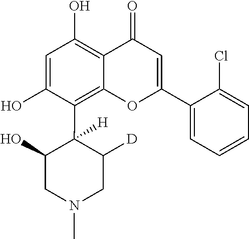 [SMILES: [2H]C1CN(C)C[C@@H](O)[C@H]1c1c(O)cc(O)c2c(=O)cc(-c3ccccc3Cl)oc12] |
| I-9 | 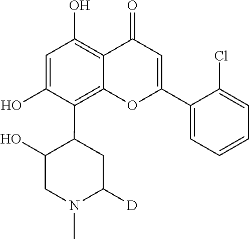 [SMILES: [2H]C1CC(c2c(O)cc(O)c3c(=O)cc(-c4ccccc4Cl)oc23)C(O)CN1C] |
| I″-9 | 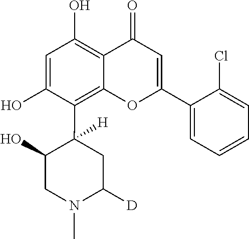 [SMILES: [2H]C1C[C@H](c2c(O)cc(O)c3c(=O)cc(-c4ccccc4Cl)oc23)[C@H](O)CN1C] |
| I-10 | 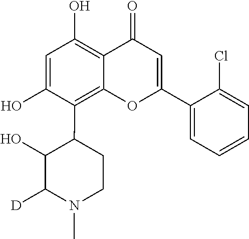 [SMILES: [2H]C1C(O)C(c2c(O)cc(O)c3c(=O)cc(-c4ccccc4Cl)oc23)CCN1C] |
| I″-10 | 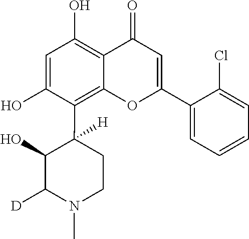 [SMILES: [2H]C1[C@@H](O)[C@@H](c2c(O)cc(O)c3c(=O)cc(-c4ccccc4Cl)oc23)CCN1C] |
| I-11 | 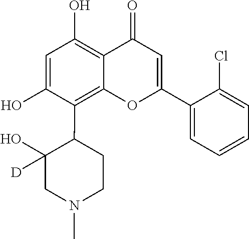 [SMILES: [2H]C1(O)CN(C)CCC1c1c(O)cc(O)c2c(=O)cc(-c3ccccc3Cl)oc12] |
| I″-11 | 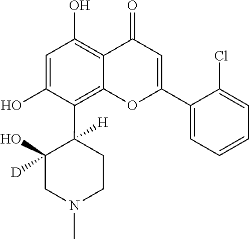 [SMILES: [2H][C@@]1(O)CN(C)CC[C@@H]1c1c(O)cc(O)c2c(=O)cc(-c3ccccc3Cl)oc12] |
| I-12 | 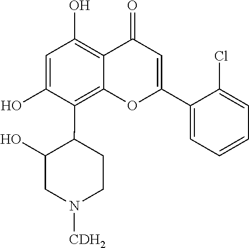 |
| I″-12 | 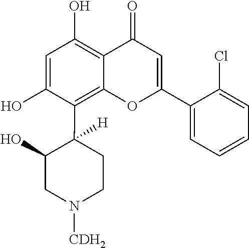 |
| I-13 | 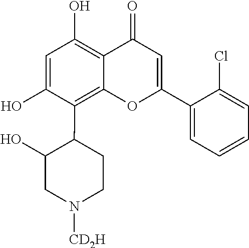 |
| I″-13 | 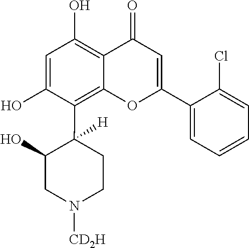 |
| I-14 | 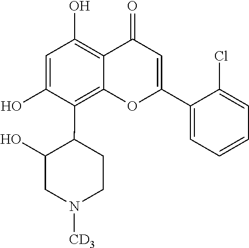 [SMILES: [2H]C([2H])([2H])N1CCC(c2c(O)cc(O)c3c(=O)cc(-c4ccccc4Cl)oc23)C(O)C1] |
| I″-14 | 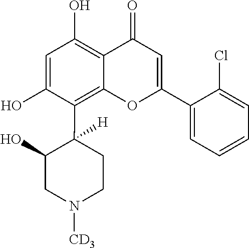 [SMILES: [2H]C([2H])([2H])N1CC[C@H](c2c(O)cc(O)c3c(=O)cc(-c4ccccc4Cl)oc23)[C@H](O)C1] |

In some other embodiments, the compound is selected from Table 2 below.

TABLE 2
Exemplary compounds of Structure (I)

| No. | Structure |
|---|---|
| II-1 | 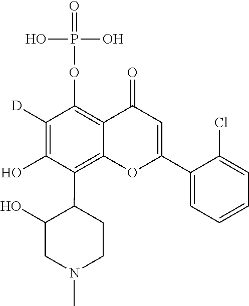 [SMILES: [2H]c1c(O)c(C2CCN(C)CC2O)c2oc(-c3ccccc3Cl)cc(=O)c2c1OP(=O)(O)O] |
| IA-1 | 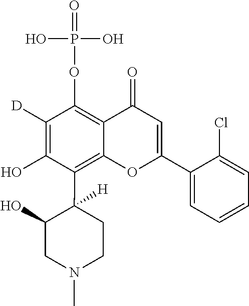 [SMILES: [2H]c1c(O)c([C@H]2CCN(C)C[C@H]2O)c2oc(-c3ccccc3Cl)cc(=O)c2c1OP(=O)(O)O] |
| II-2 | 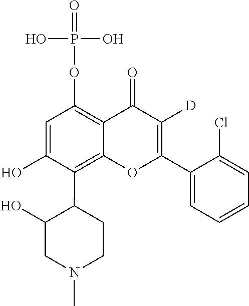 [SMILES: [2H]c1c(-c2ccccc2Cl)oc2c(C3CCN(C)CC3O)c(O)cc(OP(=O)(O)O)c2c1=O] |
| IA-2 | 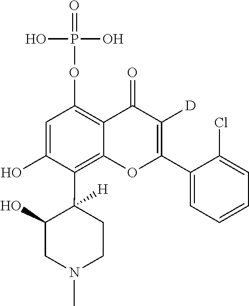 [SMILES: [2H]c1c(-c2ccccc2Cl)oc2c([C@H]3CCN(C)C[C@H]3O)c(O)cc(OP(=O)(O)O)c2c1=O] |
| II-3 | 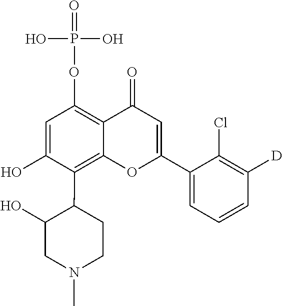 [SMILES: [2H]c1cccc(-c2cc(=O)c3c(OP(=O)(O)O)cc(O)c(C4CCN(C)CC4O)c3o2)c1Cl] |
| IA-3 | 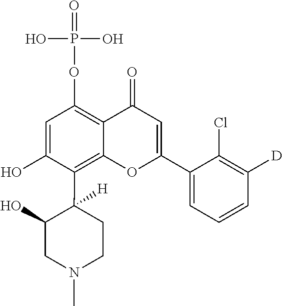 [SMILES: [2H]c1cccc(-c2cc(=O)c3c(OP(=O)(O)O)cc(O)c([C@H]4CCN(C)C[C@H]4O)c3o2)c1Cl] |
| II-4 | 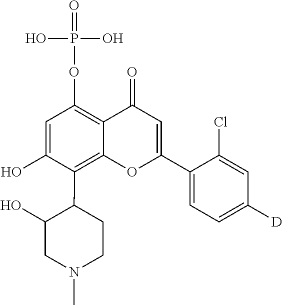 [SMILES: [2H]c1ccc(-c2cc(=O)c3c(OP(=O)(O)O)cc(O)c(C4CCN(C)CC4O)c3o2)c(Cl)c1] |
| IA-4 | 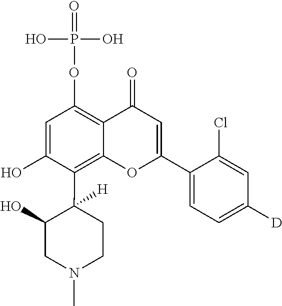 [SMILES: [2H]c1ccc(-c2cc(=O)c3c(OP(=O)(O)O)cc(O)c([C@H]4CCN(C)C[C@H]4O)c3o2)c(Cl)c1] |
| II-5 | 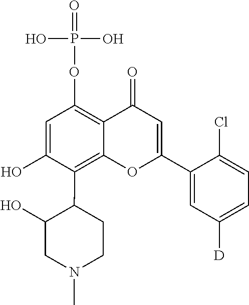 [SMILES: [2H]c1ccc(Cl)c(-c2cc(=O)c3c(OP(=O)(O)O)cc(O)c(C4CCN(C)CC4O)c3o2)c1] |
| IA-5 | 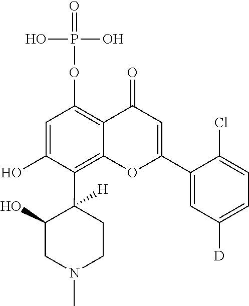 [SMILES: [2H]c1ccc(Cl)c(-c2cc(=O)c3c(OP(=O)(O)O)cc(O)c([C@H]4CCN(C)C[C@H]4O)c3o2)c1] |
| II-6 | 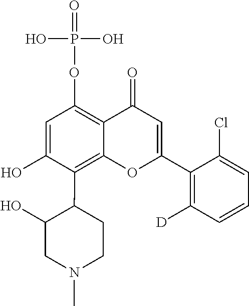 [SMILES: [2H]c1cccc(Cl)c1-c1cc(=O)c2c(OP(=O)(O)O)cc(O)c(C3CCN(C)CC3O)c2o1] |
| IA-6 | 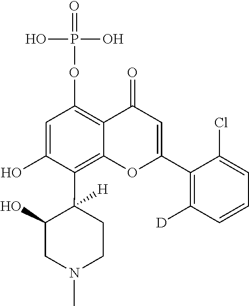 [SMILES: [2H]c1cccc(Cl)c1-c1cc(=O)c2c(OP(=O)(O)O)cc(O)c([C@H]3CCN(C)C[C@H]3O)c2o1] |
| II-7 | 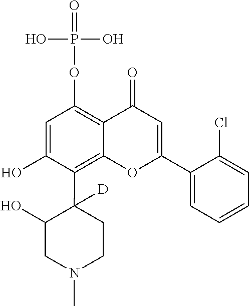 [SMILES: [2H]C1(c2c(O)cc(OP(=O)(O)O)c3c(=O)cc(-c4ccccc4Cl)oc23)CCN(C)CC1O] |
| IA-7 | 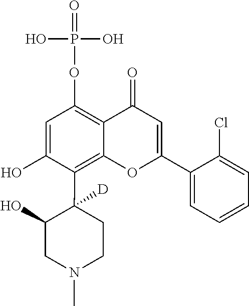 [SMILES: [2H][C@]1(c2c(O)cc(OP(=O)(O)O)c3c(=O)cc(-c4ccccc4Cl)oc23)CCN(C)C[C@H]1O] |
| II-8 | 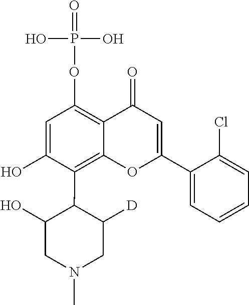 [SMILES: [2H]C1CN(C)CC(O)C1c1c(O)cc(OP(=O)(O)O)c2c(=O)cc(-c3ccccc3Cl)oc12] |
| IA-8 | 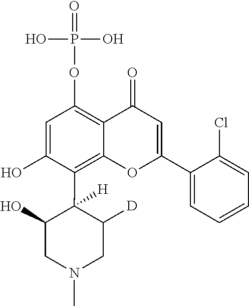 [SMILES: [2H]C1CN(C)C[C@@H](O)[C@H]1c1c(O)cc(OP(=O)(O)O)c2c(=O)cc(-c3ccccc3Cl)oc12] |
| II-9 | 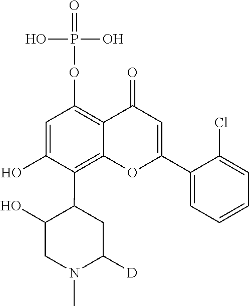 [SMILES: [2H]C1CC(c2c(O)cc(OP(=O)(O)O)c3c(=O)cc(-c4ccccc4Cl)oc23)C(O)CN1C] |
| IA-9 | 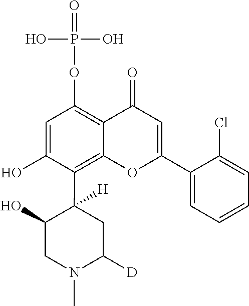 [SMILES: [2H]C1C[C@H](c2c(O)cc(OP(=O)(O)O)c3c(=O)cc(-c4ccccc4Cl)oc23)[C@H](O)CN1C] |
| II-10 | 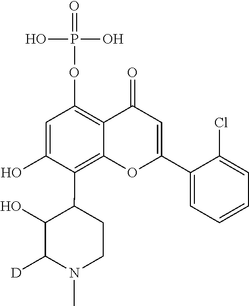 [SMILES: [2H]C1C(O)C(c2c(O)cc(OP(=O)(O)O)c3c(=O)cc(-c4ccccc4Cl)oc23)CCN1C] |
| IA-10 | 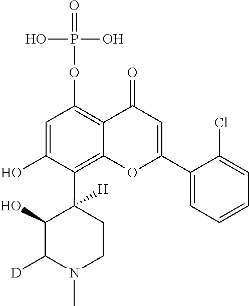 [SMILES: [2H]C1[C@@H](O)[C@@H](c2c(O)cc(OP(=O)(O)O)c3c(=O)cc(-c4ccccc4Cl)oc23)CCN1C] |
| II-11 | 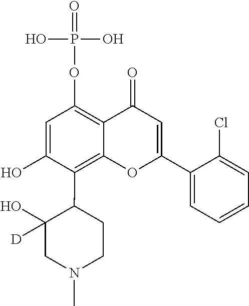 [SMILES: [2H]C1(O)CN(C)CCC1c1c(O)cc(OP(=O)(O)O)c2c(=O)cc(-c3ccccc3Cl)oc12] |
| IA-11 | 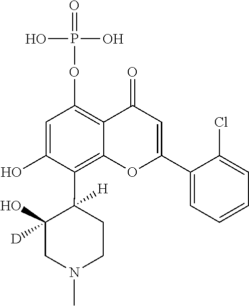 [SMILES: [2H][C@@]1(O)CN(C)CC[C@@H]1c1c(O)cc(OP(=O)(O)O)c2c(=O)cc(-c3ccccc3Cl)oc12] |
| II-12 | 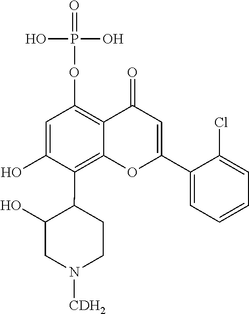 |
| IA-12 | 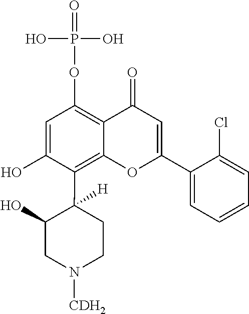 |
| II-13 | 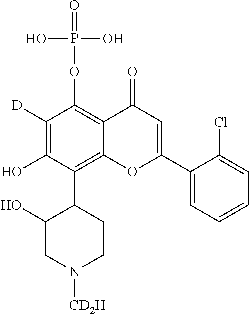 |
| IA-13 | 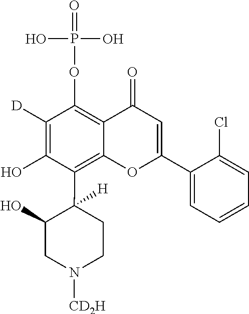 |
| II-14 | 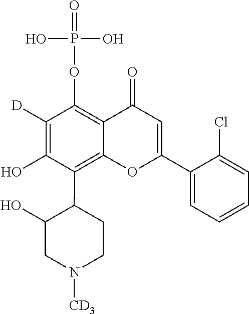 [SMILES: [2H]c1c(O)c(C2CCN(C([2H])([2H])[2H])CC2O)c2oc(-c3ccccc3Cl)cc(=O)c2c1OP(=O)(O)O] |
| IA-14 | 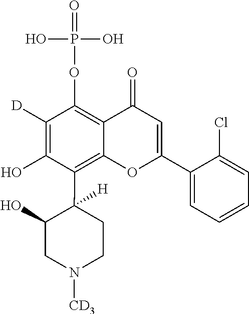 [SMILES: [2H]c1c(O)c([C@H]2CCN(C([2H])([2H])[2H])C[C@H]2O)c2oc(-c3ccccc3Cl)cc(=O)c2c1OP(=O)(O)O] |
| II-15 | 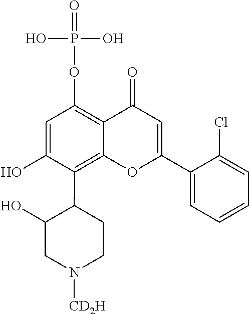 |
| IA-15 | 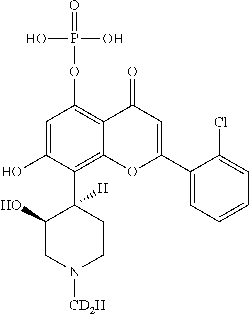 |
| II-16 | 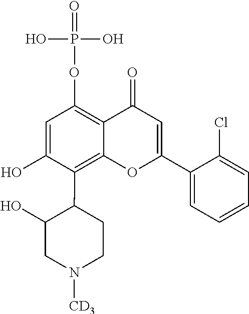 [SMILES: [2H]C([2H])([2H])N1CCC(c2c(O)cc(OP(=O)(O)O)c3c(=O)cc(-c4ccccc4Cl)oc23)C(O)C1] |
| IA-16 | 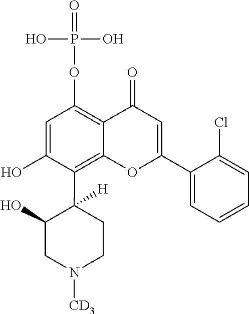 [SMILES: [2H]C([2H])([2H])N1CC[C@H](c2c(O)cc(OP(=O)(O)O)c3c(=O)cc(-c4ccccc4Cl)oc23)[C@H](O)C1] |

In certain embodiments, the compound is selected from Table 3 below.

TABLE 3
Exemplary compounds of Structure (I)

| No. | Structure |
|---|---|
| III-1 | 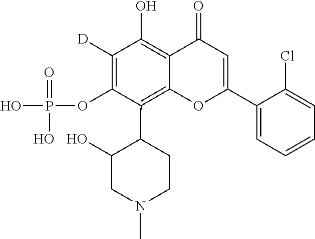 [SMILES: [2H]c1c(OP(=O)(O)O)c(C2CCN(C)CC2O)c2oc(-c3ccccc3Cl)cc(=O)c2c1O] |
| IB-1 | 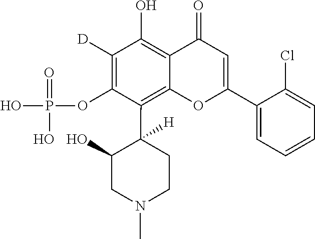 [SMILES: [2H]c1c(OP(=O)(O)O)c([C@H]2CCN(C)C[C@H]2O)c2oc(-c3ccccc3Cl)cc(=O)c2c1O] |
| III-2 | 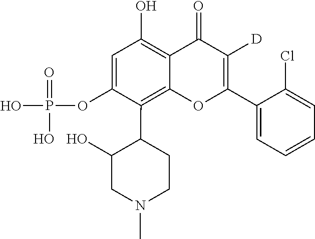 [SMILES: [2H]c1c(-c2ccccc2Cl)oc2c(C3CCN(C)CC3O)c(OP(=O)(O)O)cc(O)c2c1=O] |
| IB-2 | 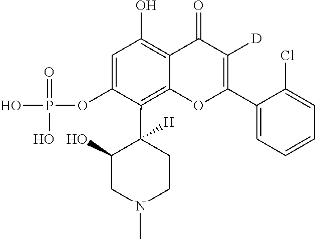 [SMILES: [2H]c1c(-c2ccccc2Cl)oc2c([C@H]3CCN(C)C[C@H]3O)c(OP(=O)(O)O)cc(O)c2c1=O] |
| III-3 | 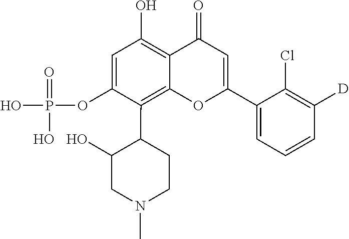 [SMILES: [2H]c1cccc(-c2cc(=O)c3c(O)cc(OP(=O)(O)O)c(C4CCN(C)CC4O)c3o2)c1Cl] |
| III′-3 | 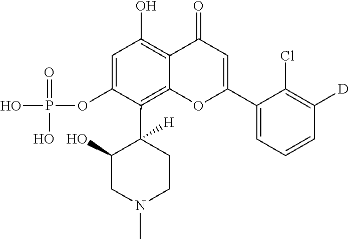 [SMILES: [2H]c1cccc(-c2cc(=O)c3c(O)cc(OP(=O)(O)O)c([C@H]4CCN(C)C[C@H]4O)c3o2)c1Cl] |
| III-4 | 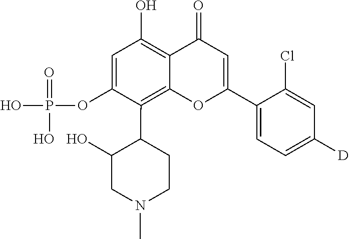 [SMILES: [2H]c1ccc(-c2cc(=O)c3c(O)cc(OP(=O)(O)O)c(C4CCN(C)CC4O)c3o2)c(Cl)c1] |
| III′-4 | 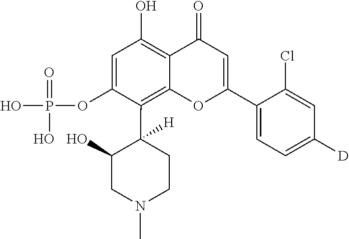 [SMILES: [2H]c1ccc(-c2cc(=O)c3c(O)cc(OP(=O)(O)O)c([C@H]4CCN(C)C[C@H]4O)c3o2)c(Cl)c1] |
| III-5 | 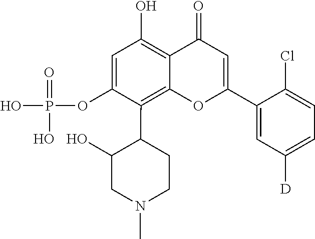 [SMILES: [2H]c1ccc(Cl)c(-c2cc(=O)c3c(O)cc(OP(=O)(O)O)c(C4CCN(C)CC4O)c3o2)c1] |
| III′-5 | 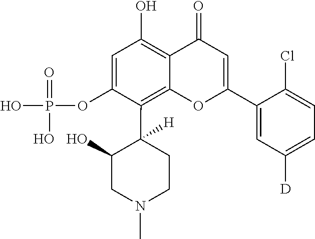 [SMILES: [2H]c1ccc(Cl)c(-c2cc(=O)c3c(O)cc(OP(=O)(O)O)c([C@H]4CCN(C)C[C@H]4O)c3o2)c1] |
| III-6 | 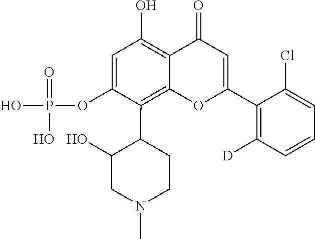 [SMILES: [2H]c1cccc(Cl)c1-c1cc(=O)c2c(O)cc(OP(=O)(O)O)c(C3CCN(C)CC3O)c2o1] |
| III′-6 | 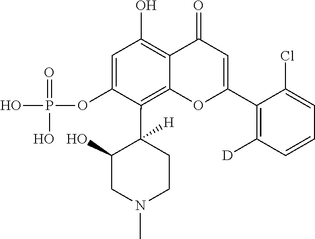 [SMILES: [2H]c1cccc(Cl)c1-c1cc(=O)c2c(O)cc(OP(=O)(O)O)c([C@H]3CCN(C)C[C@H]3O)c2o1] |
| III-7 | 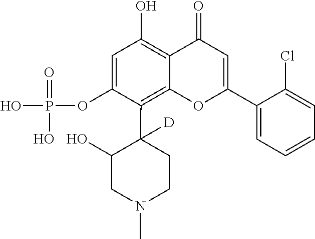 [SMILES: [2H]C1(c2c(OP(=O)(O)O)cc(O)c3c(=O)cc(-c4ccccc4Cl)oc23)CCN(C)CC1O] |
| III′-7 | 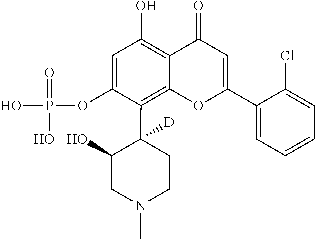 [SMILES: [2H][C@]1(c2c(OP(=O)(O)O)cc(O)c3c(=O)cc(-c4ccccc4Cl)oc23)CCN(C)C[C@H]1O] |
| III-8 | 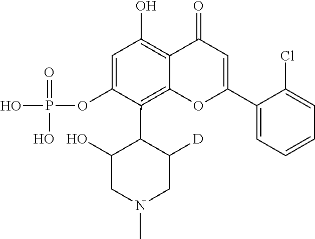 [SMILES: [2H]C1CN(C)CC(O)C1c1c(OP(=O)(O)O)cc(O)c2c(=O)cc(-c3ccccc3Cl)oc12] |
| III′-8 | 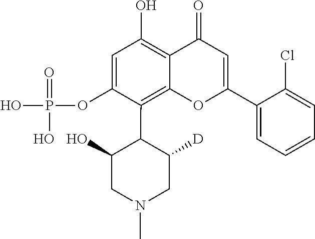 [SMILES: [2H][C@@H]1CN(C)C[C@@H](O)C1c1c(OP(=O)(O)O)cc(O)c2c(=O)cc(-c3ccccc3Cl)oc12] |
| III-9 | 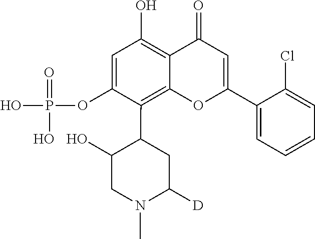 [SMILES: [2H]C1CC(c2c(OP(=O)(O)O)cc(O)c3c(=O)cc(-c4ccccc4Cl)oc23)C(O)CN1C] |
| III′-9 | 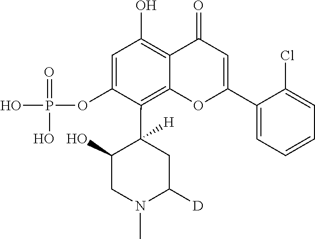 [SMILES: [2H]C1C[C@H](c2c(OP(=O)(O)O)cc(O)c3c(=O)cc(-c4ccccc4Cl)oc23)[C@H](O)CN1C] |
| III-10 | 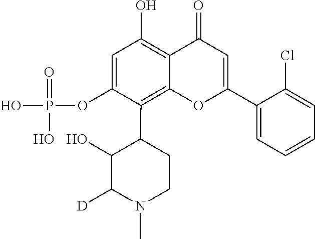 [SMILES: [2H]C1C(O)C(c2c(OP(=O)(O)O)cc(O)c3c(=O)cc(-c4ccccc4Cl)oc23)CCN1C] |
| III′-10 | 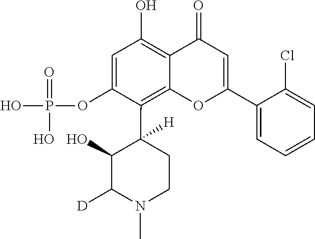 [SMILES: [2H]C1[C@@H](O)[C@@H](c2c(OP(=O)(O)O)cc(O)c3c(=O)cc(-c4ccccc4Cl)oc23)CCN1C] |
| III-11 | 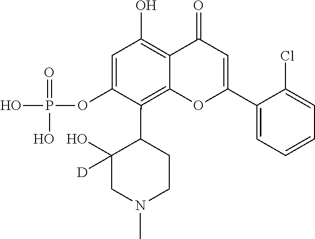 [SMILES: [2H]C1(O)CN(C)CCC1c1c(OP(=O)(O)O)cc(O)c2c(=O)cc(-c3ccccc3Cl)oc12] |
| IB-11 | 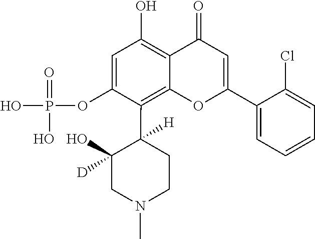 [SMILES: [2H][C@@]1(O)CN(C)CC[C@@H]1c1c(OP(=O)(O)O)cc(O)c2c(=O)cc(-c3ccccc3Cl)oc12] |
| III-12 | 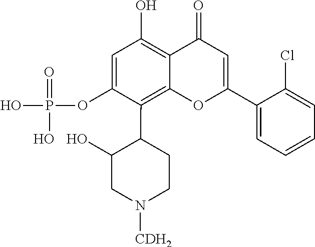 |
| IB-12 | 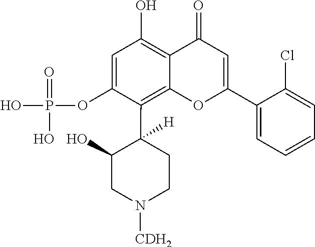 |
| III-13 | 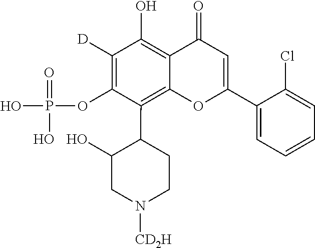 |
| IB-13 | 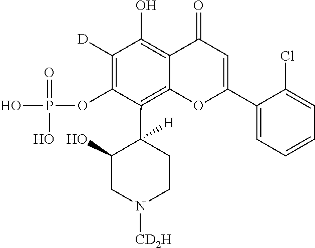 |
| III-14 | 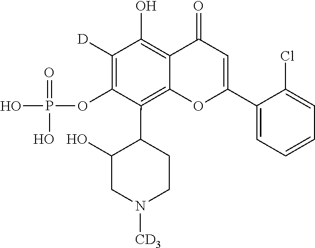 [SMILES: [2H]c1c(OP(=O)(O)O)c(C2CCN(C([2H])([2H])[2H])CC2O)c2oc(-c3ccccc3Cl)cc(=O)c2c1O] |
| IB-14 | 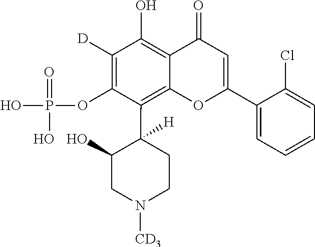 [SMILES: [2H]c1c(OP(=O)(O)O)c([C@H]2CCN(C([2H])([2H])[2H])C[C@H]2O)c2oc(-c3ccccc3Cl)cc(=O)c2c1O] |
| III-15 | 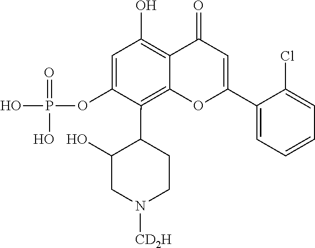 |
| IB-15 | 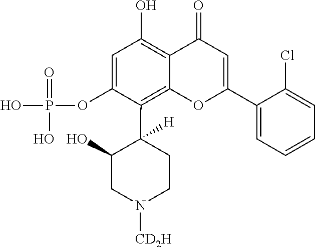 |
| III-16 | 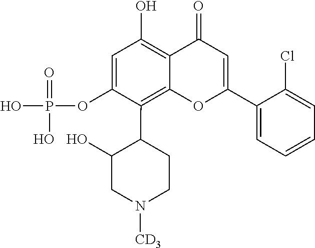 [SMILES: [2H]C([2H])([2H])N1CCC(c2c(OP(=O)(O)O)cc(O)c3c(=O)cc(-c4ccccc4Cl)oc23)C(O)C1] |
| IB-16 | 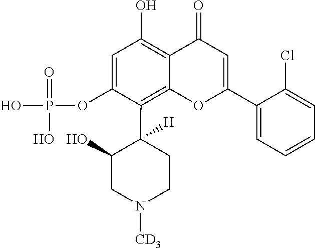 [SMILES: [2H]C([2H])([2H])N1CC[C@H](c2c(OP(=O)(O)O)cc(O)c3c(=O)cc(-c4ccccc4Cl)oc23)[C@H](O)C1] |

In certain other embodiments, the compound is selected from Table 4 below.

TABLE 4
Exemplary compounds of Structure (I)

| No. | Structure |
|---|---|
| IV-1 | 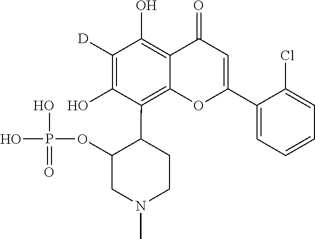 [SMILES: [2H]c1c(O)c(C2CCN(C)CC2OP(=O)(O)O)c2oc(-c3ccccc3Cl)cc(=O)c2c1O] |
| IC-1 | 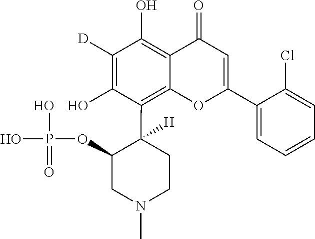 [SMILES: [2H]c1c(O)c([C@H]2CCN(C)C[C@H]2OP(=O)(O)O)c2oc(-c3ccccc3Cl)cc(=O)c2c1O] |
| IV-2 | 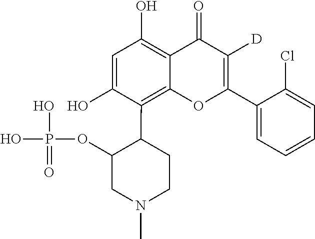 [SMILES: [2H]c1c(-c2ccccc2Cl)oc2c(C3CCN(C)CC3OP(=O)(O)O)c(O)cc(O)c2c1=O] |
| IC-2 | 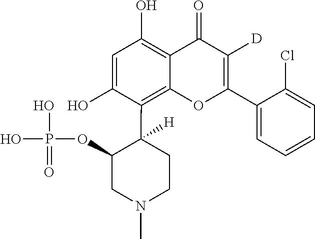 [SMILES: [2H]c1c(-c2ccccc2Cl)oc2c([C@H]3CCN(C)C[C@H]3OP(=O)(O)O)c(O)cc(O)c2c1=O] |
| IV-3 | 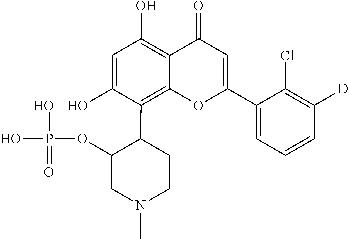 [SMILES: [2H]c1cccc(-c2cc(=O)c3c(O)cc(O)c(C4CCN(C)CC4OP(=O)(O)O)c3o2)c1Cl] |
| IC-3 | 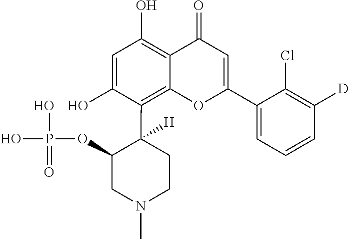 [SMILES: [2H]c1cccc(-c2cc(=O)c3c(O)cc(O)c([C@H]4CCN(C)C[C@H]4OP(=O)(O)O)c3o2)c1Cl] |
| IV-4 | 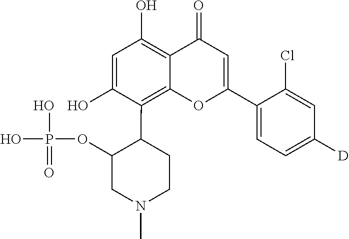 [SMILES: [2H]c1ccc(-c2cc(=O)c3c(O)cc(O)c(C4CCN(C)CC4OP(=O)(O)O)c3o2)c(Cl)c1] |
| IC-4 | 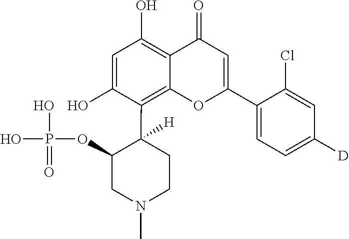 [SMILES: [2H]c1ccc(-c2cc(=O)c3c(O)cc(O)c([C@H]4CCN(C)C[C@H]4OP(=O)(O)O)c3o2)c(Cl)c1] |
| IV-5 | 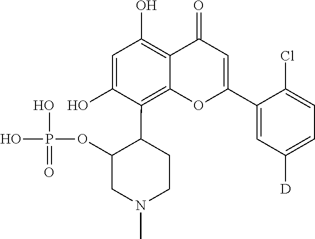 [SMILES: [2H]c1ccc(Cl)c(-c2cc(=O)c3c(O)cc(O)c(C4CCN(C)CC4OP(=O)(O)O)c3o2)c1] |
| IC-5 | 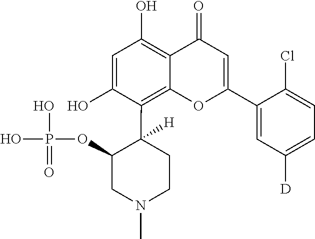 [SMILES: [2H]c1ccc(Cl)c(-c2cc(=O)c3c(O)cc(O)c([C@H]4CCN(C)C[C@H]4OP(=O)(O)O)c3o2)c1] |
| IV-6 | 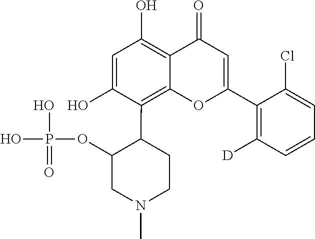 [SMILES: [2H]c1cccc(Cl)c1-c1cc(=O)c2c(O)cc(O)c(C3CCN(C)CC3OP(=O)(O)O)c2o1] |
| IC-6 | 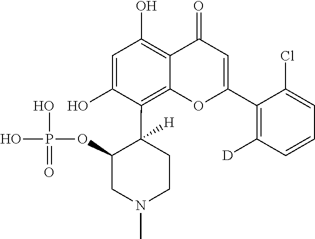 [SMILES: [2H]c1cccc(Cl)c1-c1cc(=O)c2c(O)cc(O)c([C@H]3CCN(C)C[C@H]3OP(=O)(O)O)c2o1] |
| IV-7 | 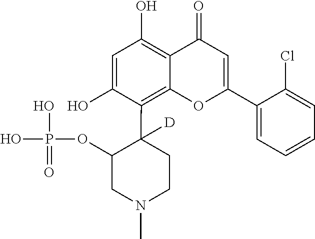 [SMILES: [2H]C1(c2c(O)cc(O)c3c(=O)cc(-c4ccccc4Cl)oc23)CCN(C)CC1OP(=O)(O)O] |
| IC-7 | 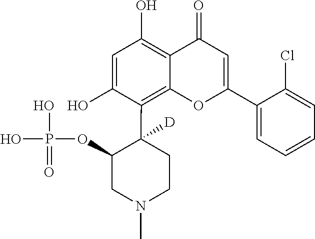 [SMILES: [2H][C@]1(c2c(O)cc(O)c3c(=O)cc(-c4ccccc4Cl)oc23)CCN(C)C[C@H]1OP(=O)(O)O] |
| IV-8 | 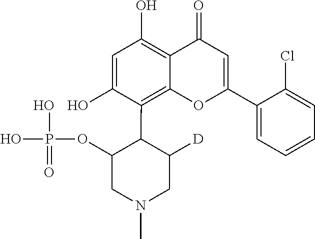 [SMILES: [2H]C1CN(C)CC(OP(=O)(O)O)C1c1c(O)cc(O)c2c(=O)cc(-c3ccccc3Cl)oc12] |
| IC-8 | 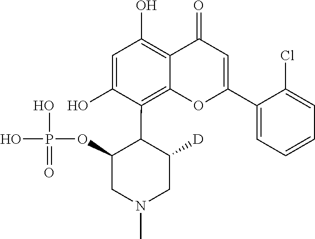 [SMILES: [2H][C@@H]1CN(C)C[C@@H](OP(=O)(O)O)C1c1c(O)cc(O)c2c(=O)cc(-c3ccccc3Cl)oc12] |
| IV-9 | 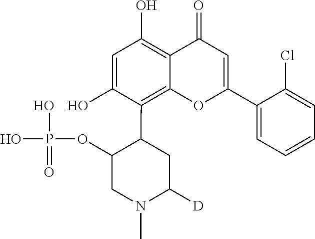 [SMILES: [2H]C1CC(c2c(O)cc(O)c3c(=O)cc(-c4ccccc4Cl)oc23)C(OP(=O)(O)O)CN1C] |
| IC-9 | 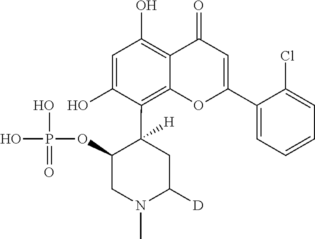 [SMILES: [2H]C1C[C@H](c2c(O)cc(O)c3c(=O)cc(-c4ccccc4Cl)oc23)[C@H](OP(=O)(O)O)CN1C] |
| IV-10 | 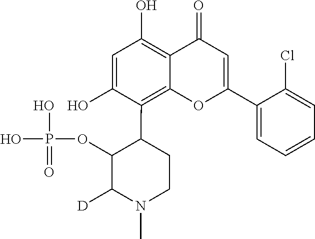 [SMILES: [2H]C1C(OP(=O)(O)O)C(c2c(O)cc(O)c3c(=O)cc(-c4ccccc4Cl)oc23)CCN1C] |
| IC-10 | 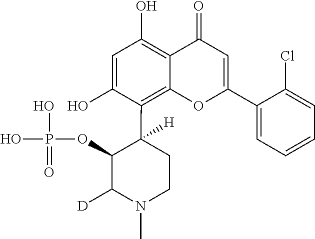 [SMILES: [2H]C1[C@@H](OP(=O)(O)O)[C@@H](c2c(O)cc(O)c3c(=O)cc(-c4ccccc4Cl)oc23)CCN1C] |
| IV-11 | 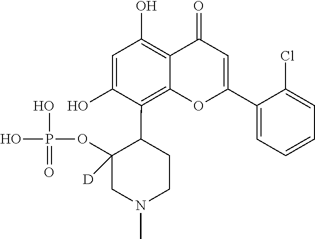 [SMILES: [2H]C1(OP(=O)(O)O)CN(C)CCC1c1c(O)cc(O)c2c(=O)cc(-c3ccccc3Cl)oc12] |
| IC-11 | 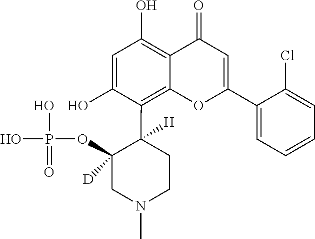 [SMILES: [2H][C@@]1(OP(=O)(O)O)CN(C)CC[C@@H]1c1c(O)cc(O)c2c(=O)cc(-c3ccccc3Cl)oc12] |
| IV-12 | 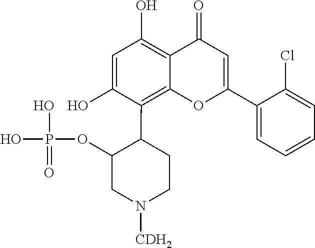 |
| IC-12 | 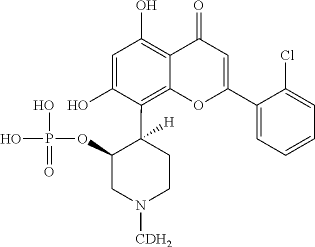 |
| IV-13 | 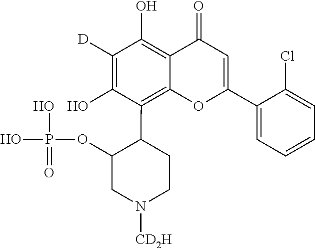 |
| IC-13 | 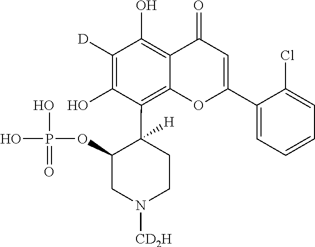 |
| IV-14 | 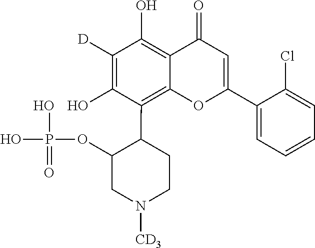 [SMILES: [2H]c1c(O)c(C2CCN(C([2H])([2H])[2H])CC2OP(=O)(O)O)c2oc(-c3ccccc3Cl)cc(=O)c2c1O] |
| IC-14 | 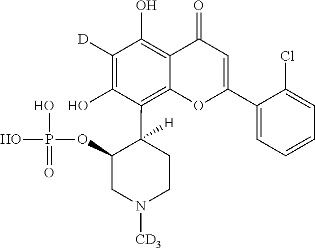 [SMILES: [2H]c1c(O)c([C@H]2CCN(C([2H])([2H])[2H])C[C@H]2OP(=O)(O)O)c2oc(-c3ccccc3Cl)cc(=O)c2c1O] |
| IV-15 | 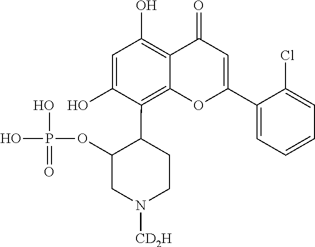 |
| IC-15 | 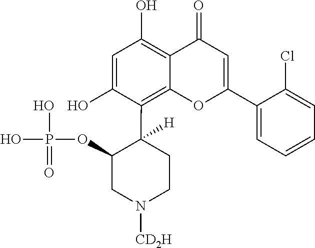 |
| IV-16 | 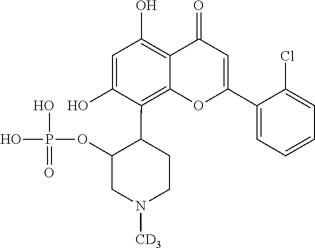 [SMILES: [2H]C([2H])([2H])N1CCC(c2c(O)cc(O)c3c(=O)cc(-c4ccccc4Cl)oc23)C(OP(=O)(O)O)C1] |
| IC-16 | 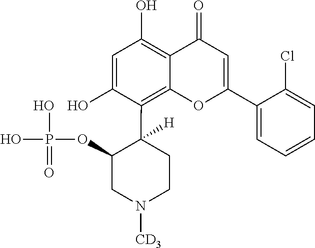 [SMILES: [2H]C([2H])([2H])N1CC[C@H](c2c(O)cc(O)c3c(=O)cc(-c4ccccc4Cl)oc23)[C@H](OP(=O)(O)O)C1] |

In further embodiments, the compound has the structure (IA), (IB) or (IC), wherein each R is H; R³ is the same at each occurrence; R⁴ is the same at each occurrence; R⁵ is the same at each occurrence; and the values for R′, R², R³, R⁴ and R⁵ are as listed in Table 5 below.

TABLE 5

| No. | R¹ | R² | R³ | R⁴ | R⁵ |
|---|---|---|---|---|---|
| 100 | D | D | H | H | H |
| 101 | D | D | D | H | H |
| 102 | D | D | D | D | H |
| 103 | D | D | D | D | D |
| 104 | H | D | D | H | H |
| 105 | H | D | D | D | H |
| 106 | H | D | D | D | D |
| 107 | H | H | D | H | H |
| 108 | H | H | D | D | H |
| 109 | H | H | D | D | D |
| 110 | D | H | D | D | D |
| 111 | H | H | H | D | H |
| 112 | H | H | H | D | D |
| 113 | D | H | H | D | D |
| 114 | D | D | H | D | D |
| 115 | D | D | H | H | D |
| 116 | D | D | D | H | D |

In certain aspects of these embodiments, the compound has the structure (IA). The compound having the structure of (IA) and the combination of substituents denoted “100” in Table 5 is referred to herein as IA-100. Thus, compound IA-100 has the following structure:

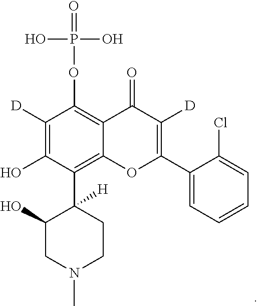 [SMILES: [2H]c1c(O)c([C@H]2CCN(C)C[C@H]2O)c2oc(-c3ccccc3Cl)c([2H])c(=O)c2c1OP(=O)(O)O]

In certain aspects of these embodiments, the compound has the structure (IB). In certain other aspects, the compound has the structure (IC). A similar naming convention to that described above for compounds having the structure (IA) and the combination of substituents denoted in Table 5 can be applied to compounds having the structure (IB) and (IC).

Atoms of this disclosure not specifically designated as a particular isotope are meant to represent any stable isotope of that atom. Unless otherwise stated, when a position is designated specifically as “H” or “hydrogen,” the position is understood to have hydrogen at its natural isotopic abundance. Unless stated otherwise, when a position is designated specifically as “D” or “deuterium,” the position is understood to have deuterium at an abundance that is at least about 3000 times greater than the natural abundance of deuterium, which is 0.015% (i.e., compounds have at least about 45% incorporation of deuterium at the indicated position).

In other embodiments, a compound of this disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Certain embodiments provide a mixture of the compounds of the foregoing embodiments. For example, one embodiment provides a mixture comprising a compound of structure (I) according to the foregoing embodiments and lighter isotopologues of the compound of structure (I), wherein at least 50% of the mixture is the compound of structure (I). In some more specific embodiments, at least 55% of the mixture is the compound of structure (I). In other specific embodiments, at least 57%, at least 60%, at least 62%, at least 65%, at least 67%, at least 70%, at least 72%, at least 75%, at least 77%, at least 80%, at least 82%, at least 85%, at least 87%, at least 90%, at least 92%, or at least 95% of the mixture is the compound of structure (I). In some other specific embodiments, at least 7%, at least 10%, at least 12%, at least 15%, at least 17%, at least 20%, at least 22%, at least 25%, at least 27%, at least 30%, at least 32%, at least 35%, at least 37%, at least 40%, at least 42%, or at least 45% of the mixture is the compound of structure (I).

In some embodiments, any of the foregoing compounds is in the form of a pharmaceutically acceptable salt. The salt may be an acid addition salt or a base addition salt, as those terms are described herein. For example, the salt may be an amine salt formed by protonation of the N-methyl piperazine moiety (e.g., HCl salt and the like). In other embodiments, the salt is formed at a phosphate group, and the compound is in the form of a mono- or di-salt of the phosphate group (e.g., mono- or disodium phosphate salt, and the like). All pharmaceutically acceptable salts of the foregoing compounds are included in the scope of the disclosure.

Also provided are pharmaceutical compositions comprising a pharmaceutically acceptable carrier or excipient and any of the foregoing compounds (i.e., a compound of structure (I), (I′), (I″), (IA), (IB) or (IC)).

Any of the carriers and/or excipients known in the art for oral formulation may be used in these embodiments, in addition to other carriers and/or excipients derivable by one of ordinary skill in the art (e.g., carriers and/or excipients known in the art for intravenous formulations).

For the purposes of administration, the compounds of the present disclosure may be administered as a raw chemical or may be formulated as pharmaceutical compositions. Embodiments of the pharmaceutical compositions of the present disclosure comprise a compound of structure (I), or a stereoisomer, tautomer or pharmaceutically acceptable salt thereof, and a pharmaceutically acceptable carrier or excipient. The compound of the disclosure (e.g., the compound of structure (I), or a stereoisomer, tautomer or pharmaceutically acceptable salt thereof) is present in the composition in an amount which is effective to treat a particular disease or condition of interest—that is, typically in an amount sufficient to treat a disease associated with overexpression of a CDK and, preferably, with acceptable toxicity to the patient. Bioavailability of compounds of the disclosure can be determined by one skilled in the art. Appropriate concentrations and dosages can be readily determined by one skilled in the art.

Administration of the compounds of the disclosure in pure form or in an appropriate pharmaceutical composition, can be carried out via any of the accepted modes of administration of agents for serving similar utilities (e.g., via intravenous administration, oral administration).

The pharmaceutical compositions disclosed herein can be prepared by combining a compound of the disclosure with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term “parenteral,” as used herein, includes subcutaneous injections, intravenous, intramuscular, intrasternal injections and/or infusion techniques. Pharmaceutical compositions described herein are formulated so as to allow the active ingredients contained therein to be bioavailable upon administration of the composition to a mammal. Compositions that will be administered to a mammal take the form of one or more dosage units, where, for example, a tablet may be a single dosage unit, and a container of a compound of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the disclosure for treatment of a disease or condition of interest in accordance with the teachings of this disclosure.

A pharmaceutical composition of some embodiments of the disclosure may be in the form of a solid or liquid. In one aspect, the carrier(s) are particulate, so that the compositions are, for example, in tablet or powder form. The carrier(s) may be liquid, with the compositions being, for example, oral syrup, injectable liquid or aerosol, which is useful in, for example, inhalatory administration.

Some embodiments provide a pharmaceutical composition formulated for oral delivery. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and/or a coloring agent.

When the pharmaceutical composition is in the form of a capsule, for example, a gelatin capsule, it may contain, in addition to materials of the above type, a liquid carrier, such as polyethylene glycol or oil.

The pharmaceutical composition may be in the form of a liquid, for example, an elixir, syrup, solution, emulsion or suspension. The liquid may be for oral administration or for delivery by injection, for example. When intended for oral administration, preferred compositions contain, in addition to a compound of the disclosure, one or more of a sweetening agent, preservative, dye/colorant and/or flavor enhancer. In a composition intended to be administered by injection, one or more of a surfactant, preservative, wetting agent, dispersing agent, suspending agent, buffer, stabilizer and/or isotonic agent may be included.

The liquid pharmaceutical compositions of some embodiments of the disclosure, whether they be solutions, suspensions or other like form, may include one or more of the following adjuvants: sterile diluents, such as water for injection, saline solution, preferably, physiological saline, Ringer's solution, isotonic sodium chloride, fixed oils, such as synthetic mono- or diglycerides, which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of certain embodiments of the disclosure intended for either parenteral or oral administration should contain an amount of a compound of the disclosure such that a suitable dosage will be obtained upon administration to a mammal.

In some embodiments, the pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents, such as water and alcohol, and/or emulsifiers and/or stabilizers. Thickening agents may be present in a pharmaceutical composition for topical administration. If intended for transdermal administration, the composition may include a transdermal patch or iontophoresis device.

The pharmaceutical composition of various embodiments of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

Embodiments of the pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of some embodiments of the disclosure in solid or liquid form may include an agent that binds to the compound of the disclosure and thereby assists in the delivery of the compound. Suitable agents that may act in this capacity include a monoclonal or polyclonal antibody, a protein or a liposome.

The pharmaceutical composition of other embodiments of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the disclosure may be delivered in single phase, bi-phasic, or tri-phasic systems in order to deliver the active ingredient(s). Delivery of the aerosol includes the necessary container, activators, valves, sub-containers, and the like, which together may form a kit. One skilled in the art, without undue experimentation may determine preferred aerosols.

In some embodiments, the pharmaceutical compositions of the disclosure may be prepared by methodology well known in the pharmaceutical art. For example, a pharmaceutical composition intended to be administered by injection can be prepared by combining a compound of the disclosure with sterile, distilled water so as to form a solution. A surfactant may be added to facilitate the formation of a homogeneous solution or suspension. Surfactants are compounds that non-covalently interact with the compound of the disclosure so as to facilitate dissolution or homogeneous suspension of the compound of the disclosure in the aqueous delivery system.

The compounds of the disclosure are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the disclosure may also be administered simultaneously with, prior to, or after administration of one or more other therapeutic agents (e.g., an effective amount of one or more other therapeutic agents). Such combination therapy includes administration of a single pharmaceutical dosage formulation which contains a compound of the disclosure and one or more additional therapeutic agents, as well as administration of the compound of the disclosure and each additional therapeutic agent in its own separate pharmaceutical dosage formulation. For example, a compound of the disclosure and the additional therapeutic agent can be administered to a mammal together in a single oral dosage composition, such as a tablet or capsule, or each agent can be administered in separate oral dosage formulations. Alternatively, a compound of the disclosure can be administered in an intravenous or oral dosage formulation and the other therapeutic agent can be administered in a separate oral dosage formulation. Where separate dosage formulations are used, the compounds of the disclosure and one or more additional therapeutic agents can be administered at essentially the same time, i.e., concurrently, or at separately staggered times, i.e., sequentially; combination therapy is understood to include all these regimens.

Toxicity and therapeutic efficacy of compounds of the disclosure, and compositions and methods described herein can be determined by standard pharmaceutical procedures in cell cultures or experimental animals, e.g., by determining the IC₅₀ and the LD₅₀ for the administered compound. For administration, effective amounts (also referred to as doses) can be initially estimated based on results from in vitro assays and/or animal model studies. For example, a dose can be formulated in animal models to achieve a circulating concentration range that includes an IC₅₀ as determined in cell culture against a particular target. The dosage may vary depending upon the dosage form employed and the route of administration utilized. The exact formulation, route of administration and dosage can be chosen by the individual physician in view of the mammal's condition. (See, e.g., Goodman & Gilman's The Pharmacological Basis Of Therapeutics, Ch. 3, 9^{th} ed., Ed. by Hardman, J., and Limbard, L., McGraw-Hill, New York City, 1996, p. 46.).

In some embodiments, the concentration of the compound of the disclosure (e.g., the compound of structure (I)) provided in the pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the compound of the disclosure (e.g., the compound of structure (I)) provided in the pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the compound of the disclosure (e.g., the compound of structure (I)) provided in the pharmaceutical compositions of the present disclosure is in the range of from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of the compound of the disclosure (e.g., the compound of structure (I)) provided in the pharmaceutical composition is in the range of from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount the compound of the disclosure (e.g., the compound of structure (I)) provided in the pharmaceutical composition is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the compound of the disclosure (e.g., the compound of structure (I)) provided in the pharmaceutical composition is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of the compound of the disclosure (e.g., the compound of structure (I)) provided in the pharmaceutical composition is in the range of 0.0001-10 g, 0.0005-9 g, 0.001-8 g, 0.005-7 g, 0.01-6 g, 0.05-5 g, 0.1-4 g, 0.5-4 g, or 1-3 g. In some embodiments, the amount of the compound of the disclosure (e.g., the compound of structure (I)) provided in the pharmaceutical composition is in the range of 0.0001-1 g, 0.0005-0.5 g, 0.0005-0.01 g, or 0.001-0.01 g.

It will also be appreciated by those skilled in the art that, in the processes for preparing compounds of structure (I) described herein, the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” All prodrugs of compounds of this disclosure are included within the scope of the disclosure.

Furthermore, all compounds of the disclosure which exist in free base or acid form can be converted to their pharmaceutically acceptable salts by treatment with the appropriate inorganic or organic base or acid by methods known to one skilled in the art. Salts of the compounds of the disclosure can also be converted to their free base or acid form by standard techniques.

For example, certain embodiments of compounds of structure (I) can be prepared by addition of a phosphate group to one of three free hydroxyls of deuterated alvocidib. A suitable method for synthesizing compounds of structure (I) by addition of a phosphate group to one of three free hydroxyls is disclosed in U.S. Patent Appln. Publication No. US 2016/0340376, and depicted in Scheme 1.

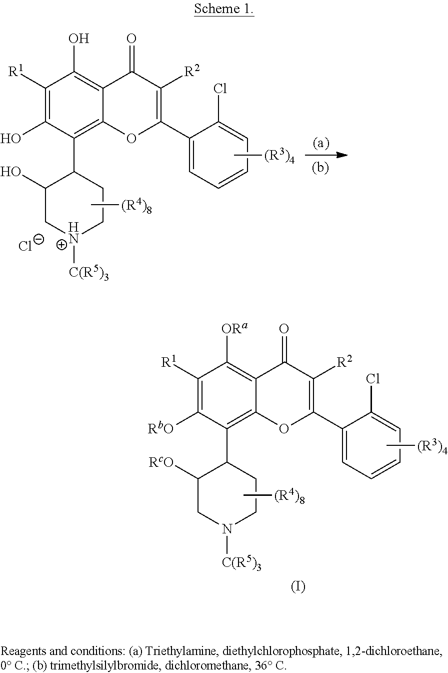

In a manner analogous to that disclosed in US 2016/0340376, an appropriately deuterated alvocidib hydrochloride salt is reacted with an appropriately protected chlorophosphate, such as diethylchlorophosphate. Alternatively, in a manner analogous to that disclosed in International Publication No. WO 2018/094275 appropriately deuterated alvocidib (as opposed to the hydrochloride salt depicted in Scheme 1) can be reacted with (EtO)₂POH and iodine instead of diethylchlorophosphate and triethylamine in the reaction corresponding to step (a) in Scheme 1, to obtain an appropriately deuterated compound of structure (I), wherein at least one (e.g., one, two or three) of R^{a}, R^{b} and R^{c} is —P(═O)(OH)₂, and the remaining of R^{a}, R^{b} and R^{c} (if any) are each H.

Deprotection of the resulting phosphate(s) then provides the appropriately deuterated compound of structure (I), wherein at least one (e.g., one, two or three) of R^{a}, R^{b} and R^{c} is —P(═O)(OH)₂, and the remaining of R^{a}, R^{b} and R^{c} (if any) are each H. It will be apparent to one of ordinary skill in the art that compounds of structure (I) having a variety of combinations of phosphate (e.g., a single phosphate) at the three hydroxyl groups of appropriately deuterated alvocidib can be prepared according to Scheme 1, and the desired regioisomer(s) separated by usual techniques, such as chromatography.

Protecting group strategies for optimizing the yield of the desired regioisomer will also be apparent to one of ordinary skill in the art. A convenient method for protecting the oxygen atom at the position corresponding to —OR^{c} in structure (I), for example, is described in International Publication No. WO 2018/094275, and depicted in Scheme 2.

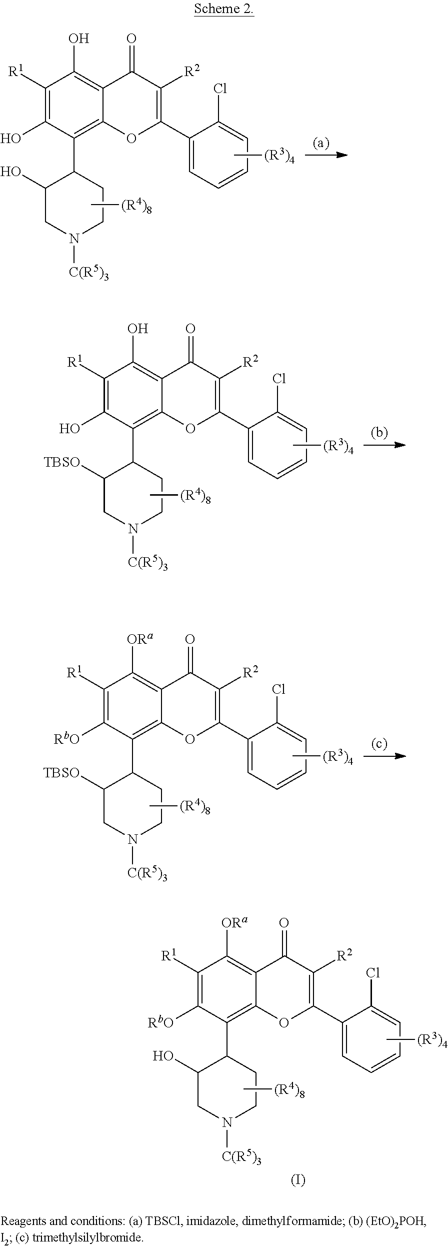

In a manner analogous to that disclosed in International Publication No. WO 2018/094275, appropriately deuterated alvocidib is reacted with tert-butyldimethylsilyl chloride (TBSCl) in the presence of an amine base, such as imidazole, to obtain an appropriately deuterated, protected alvocidib. The appropriately deuterated, protected alvocidib is then treated with (EtO)₂POH and iodine to install one or more phosphate groups. Deprotection of the protected hydroxyl and the protected phosphate group(s) in a manner analogous to that described in US 2016/0340376 yields appropriately deuterated compound of structure (I), wherein at least one (e.g., one or two) of R^{a} and R^{b} is —P(═O)(OH)₂, and the remaining of R^{a} and R^{b} (if any) are each H.

A representative synthesis of I-1 and I″-1 is depicted in Scheme 3. Steps 1-3 and 5 in Scheme 3 can be carried out in a manner analogous to that described in ChemBioChem, 10(12), 2072-2080, 2009. It is expected that deuterium can be introduced at the position corresponding to R¹ in structure I by halogen exchange, either by lithiation, palladium catalysis or a transfer Grignard reaction. Such transformations are known in the art.

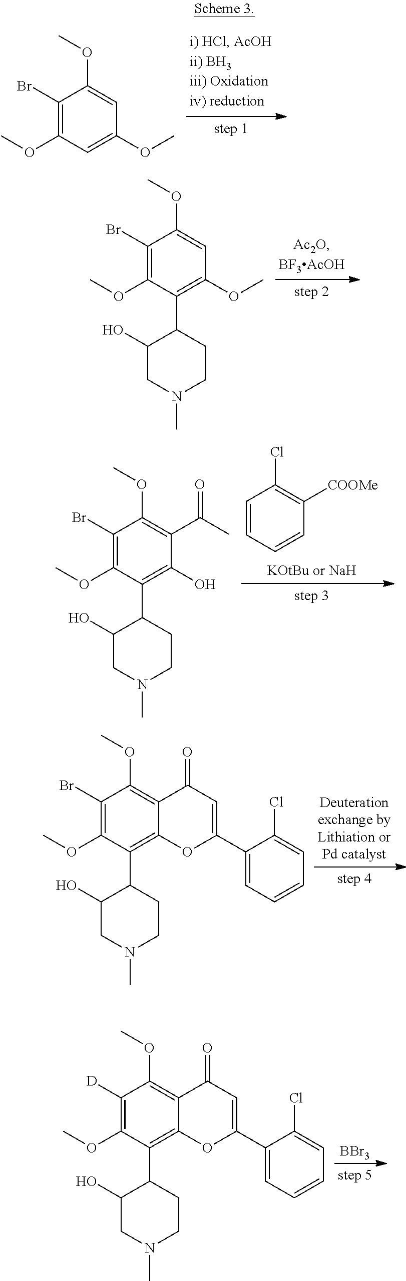

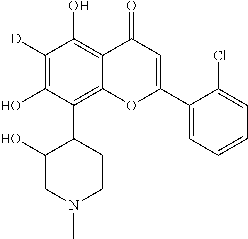 [SMILES: [2H]c1c(O)c(C2CCN(C)CC2O)c2oc(-c3ccccc3Cl)cc(=O)c2c1O]

A representative synthesis of 2-(2-chlorophenyl-3,4,5,6-d₄)-5,7-dihydroxy-8-(3-hydroxy-1-methylpiperidin-4-yl)-4H-chromen-4-one is depicted in Scheme 4. 1-(2-Hydroxy-3-(3-hydroxy-1-methylpiperidin-4-yl)-4,6-dimethoxyphenyl)ethan-1-one can be synthesized using the procedure described in ChemBioChem, 10(12), 2072-2080; 2009. Appropriately deuterated ester intermediate methyl 2-chlorobenzoate-3,4,5,6-d₄ can be synthesized using the procedure for step 5 described in the Journal of American Society for Mass Spec., 29(8), 1601-01610; 2018, and the procedure for step 6 described in Angew. Chem. Int. Ed., 52(16), 4440-4444; 2013. It is expected that treatment of 1-(2-hydroxy-3-(3-hydroxy-1-methylpiperidin-4-yl)-4,6-dimethoxyphenyl)ethan-1-one with the appropriately deuterated ester intermediate in the presence of a base, such as potassium tert-butoxide or sodium hydride, will form 2-(2-chlorophenyl-3,4,5,6-d₄)-8-(3-hydroxy-1-methylpiperidin-4-yl)-5,7-dimethoxy-4H-chromen-4-one. Removal of the methyl groups of 2-(2-chlorophenyl-3,4,5,6-d₄)-8-(3-hydroxy-1-methylpiperidin-4-yl)-5,7-dimethoxy-4H-chromen-4-one with BBr₃ is expected to yield 2-(2-chlorophenyl-3,4,5,6-d₄)-5,7-dihydroxy-8-(3-hydroxy-1-methylpiperidin-4-yl)-4H-chromen-4-one.

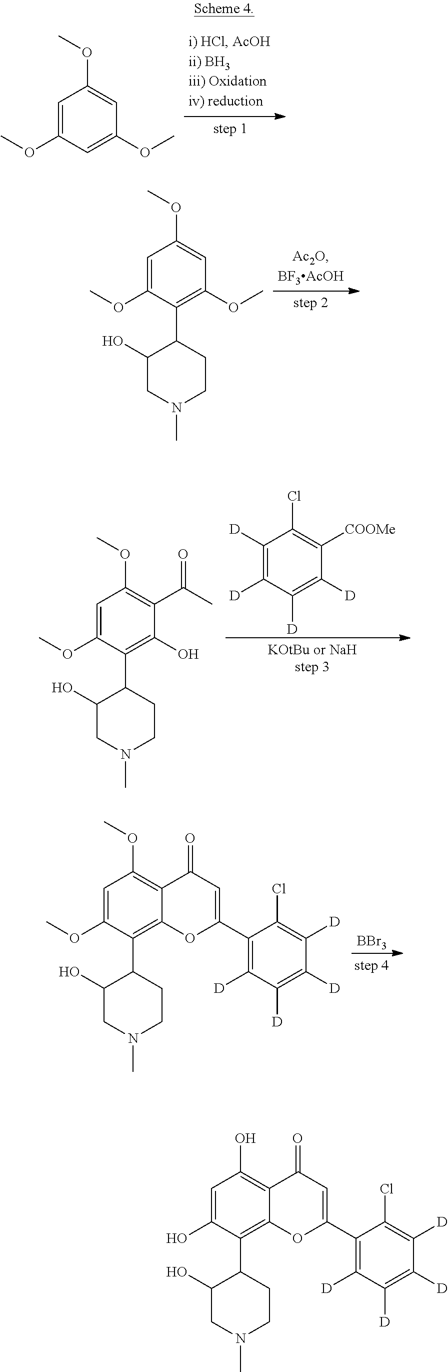

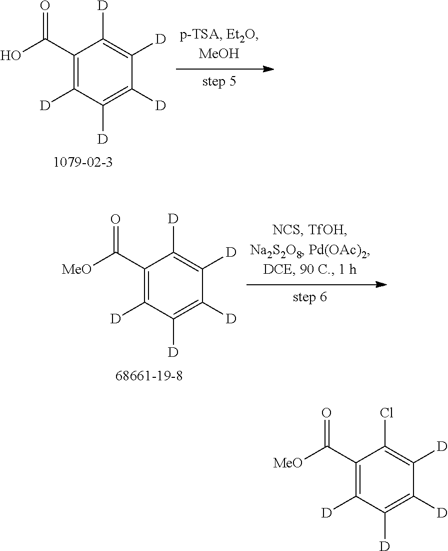 [SMILES: [2H]c1c([2H])c([2H])c(C(=O)O)c([2H])c1[2H] ; [2H]c1c([2H])c([2H])c(C(=O)OC)c([2H])c1[2H] ; [2H]c1c([2H])c([2H])c(C(=O)OC)c(Cl)c1[2H]]

A representative synthesis of 2-(2-chlorophenyl)-5,7-dihydroxy-8-(3-hydroxy-1-methylpiperidin-4-yl)-4H-chromen-4-one-3-d is depicted in Scheme 5. 1-(2-Hydroxy-3-(3-hydroxy-1-methylpiperidin-4-yl)-4,6-dimethoxyphenyl)ethan-1-one-2,2,2-d₃ can be synthesized in a manner analogous to the procedure used for the deuteration of acetophenone reported in ChemComm, 50(25), 3299-3302; 2014 by treating 1-(2-hydroxy-3-(3-hydroxy-1-methylpiperidin-4-yl)-4,6-dimethoxyphenyl)ethan-1-one with NaOD in the presence of D₂O. Treatment of 1-(2-hydroxy-3-(3-hydroxy-1-methylpiperidin-4-yl)-4,6-dimethoxyphenyl)ethan-1-one-2,2,2-d₃ and methyl 2-chlorobenzoate with a base, such as potassium tert-butoxide or sodium hydride, is expected to yield 2-(2-chlorophenyl)-8-(3-hydroxy-1-methylpiperidin-4-yl)-5,7-dimethoxy-4H-chromen-4-one-3-d, which, upon deprotection with BBr₃, is expected to yield 2-(2-chlorophenyl)-5,7-dihydroxy-8-(3-hydroxy-1-methylpiperidin-4-yl)-4H-chromen-4-one-3-d.

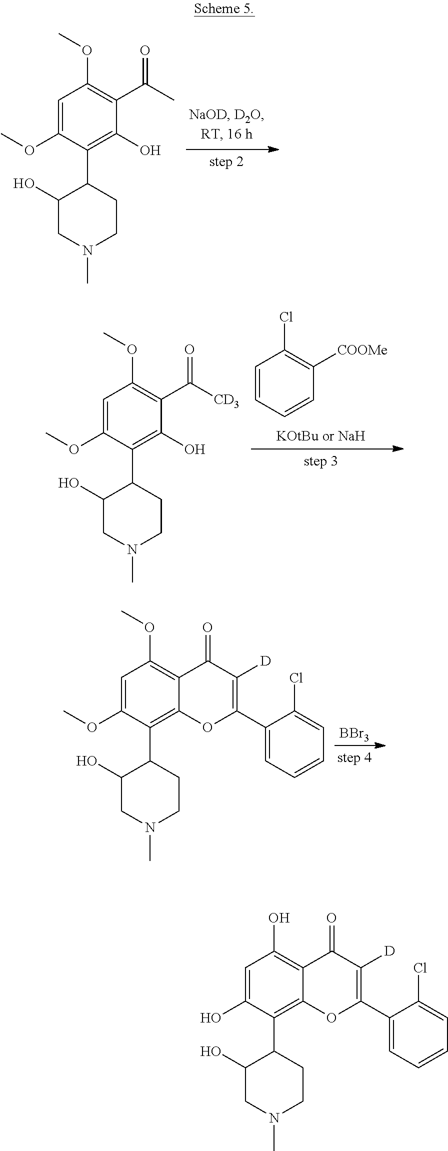

A representative synthesis of 2-(2-chlorophenyl)-5,7-dihydroxy-8-(3-hydroxy-1-methylpiperidin-4-yl-2,2,3,4,5,5,6,6-d₈)-4H-chromen-4-one is depicted in Scheme 6. 1-Methylpiperidin-4-one-2,2,3,3,5,5,6,6-d₈ can be synthesized using the procedure for steps 1-4 disclosed in U.S. Patent Appln. Publication No. US 2008/0280886. 1-Methylpiperidin-4-one-2,2,3,3,5,5,6,6-d₈ can then be used in step 5 in a manner analogous to that reported in ChemBioChem, 10(12), 2072-2080; 2009. Step 6 can also be performed in a manner analogous to that reported in ChemBioChem, 10(12), 2072-2080; 2009. Treatment of 1-(2-hydroxy-3-(3-hydroxy-1-methylpiperidin-4-yl-2,2,3,4,5,5,6,6-d₈)-4,6-dimethoxyphenyl)ethan-1-one and methyl 2-chlorobenzoate with a base, such as potassium tert-butoxide or sodium hydride, is expected to yield 2-(2-chlorophenyl)-8-(3-hydroxy-1-methylpiperidin-4-yl-2,2,3,4,5,5,6,6-d₈)-5,7-dimethoxy-4H-chromen-4-one, which, upon deprotection with BBr₃, is expected to yield 2-(2-chlorophenyl)-5,7-dihydroxy-8-(3-hydroxy-1-methylpiperidin-4-yl-2,2,3,4,5,5,6,6-d₈)-4H-chromen-4-one.

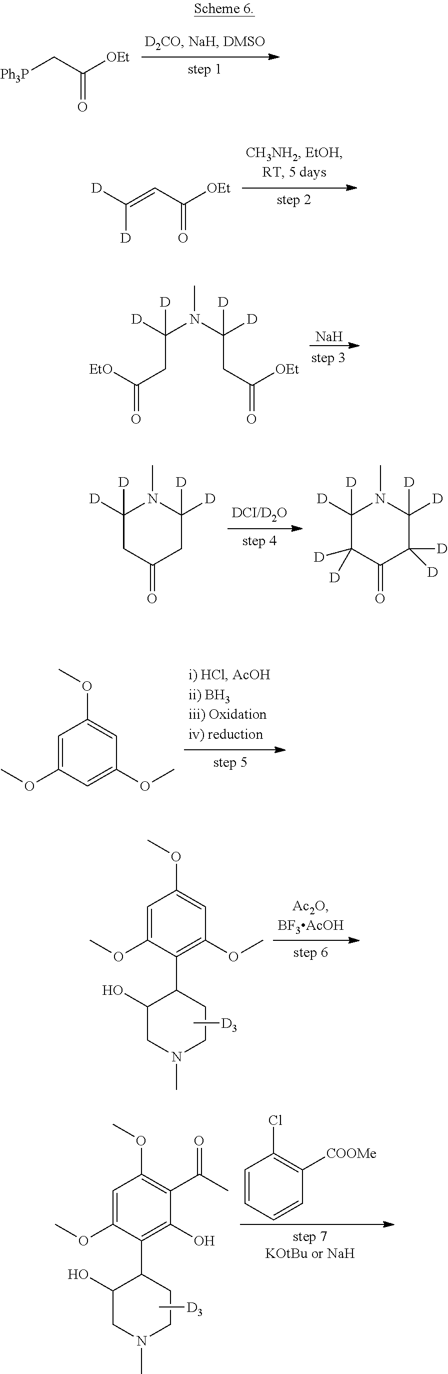

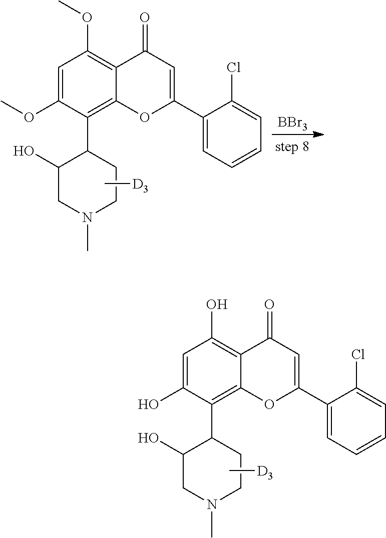

In certain embodiments, appropriately deuterated alvocidib (and salts and solvates thereof) can be purchased from commercial sources or prepared according to methods known in the art, for example, as described in U.S. Pat. Nos. 6,136,981; 6,225,473; 6,406,912; 6,576,647; 6,821,990 and 5,908,934, the full disclosures of which are herein incorporated by reference in their entireties. Such methods can be carried out using corresponding deuterated reagents and/or intermediates to synthesize the compounds of the disclosure or by invoking standard synthetic protocols known in the art for introducing isotopic atoms to a chemical structure. For example, compounds of the disclosure can be synthesized in a manner analogous to the methods presented in U.S. Pat. Nos. 6,136,981; 6,225,473; 6,406,912; 6,576,647; 6,821,990 and 5,908,934 using appropriately deuterated starting material(s).

### 1. Methods

In various embodiments, the disclosure provides a method for treating a disease in a mammal in need thereof by administration of a compound of structure (I), or a pharmaceutical composition comprising the same, to the mammal. In some specific embodiments, the method is for treating a disease associated with overexpression of a cyclin-dependent kinase (CDK) in a mammal in need thereof, the method comprising administering a therapeutically effective amount of a compound of the disclosure (e.g., any of the foregoing compounds of structure (I)), or a pharmaceutical composition comprising the same, to the mammal.

In some more embodiments, the disease is cancer, for example a hematologic cancer. In some of these embodiments, the hematologic cancer is selected from acute myelogenous leukemia (AML), multiple myeloma, follicular lymphoma, acute lymphoblastic leukemia (ALL), chronic lymphocytic leukemia (CLL) and non-Hodgkin's lymphoma. In other embodiments, the hematological cancer is acute myelogenous leukemia (AML). In other different embodiments, the hematologic cancer is chronic lymphocytic leukemia (CLL). In still more different embodiments, the hematologic cancer is myelodysplasic syndrome (MDS). In some embodiments, the hematologic cancer is acute lymphocytic leukemia, chronic lymphogenous leukemia, mantle cell lymphoma or diffuse large B-cell lymphoma.

In embodiments, the hematologic cancer is MDS. In some embodiments, the MDS is high-risk MDS. In some embodiments, the hematologic cancer is MDS, and the subject also has secondary AML (e.g., AML derived from MDS or AML arising from prior chemotherapeutic treatment(s)).

In some embodiments, the cancer is breast cancer, pancreatic cancer, renal carcinoma, colon cancer, thyroid carcinoma, colorectal cancer (e.g., BRAF, KRAS, or NRAS-mutated colorectal carcinoma), ovarian cancer, melanoma (e.g., BRAF-mutated melanoma) or lung cancer (e.g., EGFR+ non-small cell lung cancer). In some embodiments, the cancer is an advanced solid tumor (e.g., an advanced solid tumor that has progressed despite immunotherapy).

In some other specific embodiments of the foregoing methods, the method comprises orally administering the compound of the disclosure (e.g., the compound of structure (I)), or the pharmaceutical composition comprising the same, to the mammal.

In some embodiments, the method further comprises administering an additional therapeutic agent (e.g., an effective amount of an additional therapeutic agent) to the mammal.

In certain embodiments, the additional therapeutic agent is a tetracyclic anthracycline.

In some embodiments, the tetracyclic anthracycline is daunorubicin, idarubicin, or a combination thereof. In certain embodiments, the tetracyclic anthracycline is daunorubicin.

In some embodiments, the compound of the disclosure (e.g., the compound of structure (I), or a pharmaceutically acceptable salt thereof) or pharmaceutical composition thereof, and the tetracyclic anthracycline are administered sequentially. In certain of these embodiments, the tetracyclic anthracycline is administered after the compound of the disclosure (e.g., the compound of structure (I), or a pharmaceutically acceptable salt thereof) or pharmaceutical composition thereof, is administered. The treatment methods may optionally include a treatment break between administering the compound of the disclosure (e.g., the compound of structure (I), or a pharmaceutically acceptable salt thereof) or pharmaceutical composition thereof, and administering the tetracyclic anthracycline.

In some embodiments, the methods further comprise administering to the subject an effective amount of a nucleoside analog. For example, in some embodiments the nucleoside analog is a pyrimidine analog, such as cytarabine.

In some of the foregoing embodiments, the tetracyclic anthracycline and the nucleoside analog are administered sequentially. For example, in some aspects, the compound of the disclosure (e.g., the compound of structure (I), or a pharmaceutically acceptable salt thereof) or pharmaceutical composition thereof, the tetracyclic anthracycline, and the nucleoside analog are administered sequentially. In some specific aspects, the nucleoside analog is administered after the tetracyclic anthracycline is administered. In some of the foregoing embodiments, the tetracyclic anthracycline and the nucleoside analog are co-administered.

In some embodiments, a treatment regimen comprises 11 days of treatment. For example, some embodiments comprise administering the compound of the disclosure (e.g., the compound of structure (I)) to the subject daily on a first, second and third day. Optionally, the subject receives no treatment (i.e., no compound of the disclosure or composition thereof, tetracyclic anthracycline, or nucleoside analog) on a fourth day. On a fifth day of treatment, embodiments include administering the nucleoside analog daily for 7 days (days 5-11), and administering the tetracyclic anthracycline daily for 3 days (days 5-7). In various embodiments, effective amounts of these therapeutic agents (e.g., compound of or composition comprising structure (I), tetracyclic anthracycline, and/or nucleoside analog) can decrease the number of tumor cells, decrease the number of metastases, decrease tumor volume, induce apoptosis of cancer cells, induce cancer cell death, induce radio-sensitivity in cancer cells, inhibit angiogenesis near cancer cells, inhibit cancer cell proliferation, inhibit tumor growth, prevent metastasis, reduce the number of metastases, increase life expectancy, prolong a mammal's life, reduce cancer-associated pain, and/or reduce relapse or re-occurrence of the cancer following treatment.

Accordingly, in certain embodiments the disclosure provides a method for treating a cancer in a mammal, the method comprising: administering to the mammal an effective amount of each of:

a compound of the disclosure or composition thereof (e.g., a compound of or composition comprising a compound of structure (I));

daunorubicin or idarubicin; and

cytarabine,

thereby treating the cancer in the subject.

In some embodiments the compound of the disclosure or composition thereof (e.g., compound of or composition comprising a compound of structure (I)), daunorubicin or idarubicin, and cytarabine are administered sequentially. For example, some embodiments comprise administering the compound of the disclosure (e.g., compound of or composition comprising a compound of structure (I)), to the subject before the daunorubicin or idarubicin and the cytarabine.

In particular embodiments, administration of the therapeutic agents of the disclosure (e.g., a compound of or composition comprising a compound of structure (I) and tetracyclic anthracycline) to a plurality of subjects results in an increase in one or more of progression free survival, complete remission rate, event free survival, overall survival, and bridge to bone marrow transplant. A subject is considered to be in “complete remission” if less than 5% leukemic blasts are present in the subject's bone marrow. In one particular embodiment, administration of these therapeutic agents (e.g., a compound of or composition comprising a compound of structure (I) and tetracyclic anthracycline) to a mammal results in minimal residual disease (MRD; i.e., no detectable cells being present in the subject's bone marrow).

Methods of the present disclosure include methods for treating a hematologic cancer in a mammal in need thereof, the method comprising: administering a treatment regimen (e.g., an effective amount of a treatment regimen) to a mammal that received a first-line therapy that resulted in complete remission for less than one year, the mammal having a predetermined MCL-1 dependency percentage of at least 30%, the predetermined MCL-1 dependency percentage having been obtained by an in vitro method comprising:

contacting a first portion of a plurality of cancer cells with a profiling peptide comprising a cellular uptake moiety and an MCL-1 binding domain, the MCL-1 binding domain having the sequence of RPEIWMTQGLRRLGDEINAYYAR (SEQ ID NO:1) with 0-8 modifications,

wherein the treatment regimen comprises:
a compound of the disclosure (e.g., a compound of or composition comprising a compound of structure (I));
a tricyclic dione; and
a nucleoside analog.

In certain embodiments, the methods of the present disclosure include methods for treating a hematologic cancer in a mammal in need thereof, the method comprising:

selecting a mammal that received a first-line therapy that resulted in complete remission for less than one year, the mammal having a predetermined MCL-1 dependency percentage of at least 30%, the predetermined MCL-1 dependency percentage having been obtained by an in vitro method comprising:

contacting a first portion of a plurality of cancer cells with a profiling peptide comprising a cellular uptake moiety and an MCL-1 binding domain, the MCL-1 binding domain having the sequence of SEQ ID NO:1 with 0-8 modifications; and
administering a treatment regimen (e.g., an effective amount of a treatment regimen) to the subject, the treatment regimen comprising:
   a compound of the disclosure (e.g., a compound of or composition comprising a compound of structure (I));
   a tricyclic dione; and
   a nucleoside analog.

In some more specific embodiments, the tricyclic dione is mitoxantrone, and the nucleoside analog is a pyrimidine analog, such as cytarabine. In some embodiments, the tricyclic dione is mitoxantrone, pixantrone, or a combination thereof. In certain embodiments, the tricyclic dione is mitoxantrone.

In some embodiments, the nucleoside analog is a pyrimidine analog. Any suitable pyrimidine analog may be administered, such as UFT, capecitabine, gemcitabine and cytarabine, an alkyl sulfonate, e.g., busulfan, improsulfan and piposulfan. In particular embodiments, the pyrimidine analog is cytarabine. As would be understood by one of skill in the art, a pyrimidine analog is a non-naturally occurring molecule.

A “tricyclic dione” refers to a class of compounds comprising the following core structure, which is optionally substituted at all available positions:

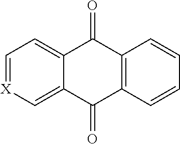

wherein X is C or N. Exemplary tricyclic diones include mitoxantrone and pixantrone.

In some such embodiments, the compound of the disclosure or composition thereof (e.g., compound of or composition comprising a compound of structure (I)) and the tricyclic dione are administered sequentially. In embodiments, the tricyclic dione and the nucleoside analog (e.g., pyrimidine analog) are administered sequentially. In embodiments, the compound of the disclosure or composition thereof (e.g., compound of or composition comprising a compound of structure (I)), the tricyclic dione, and the nucleoside analog (e.g., pyrimidine analog) are administered sequentially.

In some embodiments, the tricyclic dione is administered after the compound of the disclosure or composition thereof (e.g., compound of or composition comprising a compound of structure (I)) is administered. In other embodiments, the tricyclic dione is administered to the subject prior to administration of the compound of the disclosure or composition thereof (e.g., compound of or composition comprising a compound of structure (I)). In some embodiments, the pyrimidine analog is administered before the tricyclic dione is administered. In some embodiments, the compound of the disclosure or composition thereof (e.g., compound of or composition comprising a compound of structure (I)) is administered before the pyrimidine analog, which is administered before the tricyclic dione. In some embodiments, the administration of the pyrimidine analog and the tricyclic dione overlaps. In embodiments, the compound of the disclosure or composition thereof (e.g., compound of or composition comprising a compound of structure (I)) is administered to the subject before the tricyclic dione and the pyrimidine analog. In some embodiments, the tricyclic dione is co-administered with the pyrimidine analog.

In some embodiments, there is a treatment break between administering the compound of the disclosure or composition thereof (e.g., compound of or composition comprising a compound of structure (I)) and administering the tricyclic dione. In more specific embodiments, the treatment break between administration of the compound of the disclosure or composition thereof (e.g., compound of or composition comprising a compound of structure (I)) and the pyrimidine analog is at least about 24 to about 48 hours. In more specific embodiments, the tricyclic dione is administered more than about 24 to about 48 hours after administration of the compound of the disclosure or composition thereof (e.g., compound of or composition comprising a compound of structure (I)). In one particular embodiment, the pyrimidine analog is administered about 48 hours after administration of the compound of the disclosure or composition thereof (e.g., compound of or composition comprising a compound of structure (I)). In some embodiments, the tricyclic dione is administered at least about 120 hours after administration of the compound of the disclosure or composition thereof (e.g., compound of or composition comprising a compound of structure (I)).

In some embodiments, a treatment regimen comprises nine days of treatment. For example, some embodiments comprise administering the compound of the disclosure or composition thereof (e.g., compound of or composition comprising a compound of structure (I)) to the subject daily on a first, second and third day. Optionally, the subject receives no treatment (i.e., the compound of the disclosure or composition thereof (e.g., compound of or composition comprising a compound of structure (I)), tricyclic dione, or pyrimidine analog) on a fourth day and fifth day. Embodiments include administering the pyrimidine analog daily for three days (days six-eight) and administering the tricyclic dione daily for one day (day nine).

In various embodiments, effective amounts of these therapeutic agents (e.g., compound of or composition comprising a compound of structure (I), tricyclic dione, and/or pyrimidine analog) can decrease the number of tumor cells, decrease the number of metastases, decrease tumor volume, induce apoptosis of cancer cells, induce cancer cell death, induce radio-sensitivity in cancer cells, inhibit angiogenesis near cancer cells, inhibit cancer cell proliferation, inhibit tumor growth, prevent metastasis, reduce the number of metastases, increase life expectancy, prolong a subject's life, reduce cancer-associated pain, and/or reduce relapse or re-occurrence of the cancer following treatment.

In particular embodiments, administration of these therapeutic agents (e.g., a compound of or composition comprising a compound of structure (I), a tricyclic dione, and a pyrimidine analog) to a plurality of mammals results in an increase in one or more of progression free survival, complete remission rate, event free survival, overall survival, and bridge to bone marrow transplant relative to an untreated plurality of subjects. In some embodiments, administration of these therapeutic agents to a mammal results in complete remission. In some embodiments, administration of these therapeutic agents (e.g., a compound of or composition comprising a compound of structure (I) and tricyclic dione) to a mammal results in minimal residual disease (MRD; i.e., no detectable cells being present in the subject's bone marrow).

In certain related embodiments, methods of the disclosure further comprise administering to the mammal an effective amount of one or more additional therapeutic agents, such as one or more chemotherapeutic agents. In various embodiments, the one or more chemotherapeutic agents are one or more of alkylating agents, such as thiotepa or CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, detorubicin, 6-diazo-5-oxo-L-norleucine, adriamycin doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C” or Cytarabine); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chlorambucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-1, Raf, H-Ras, EGFR (e.g., erlotinib (Tarceva)) and VEGF-A that reduce cell proliferation; Dacogen, Velcade; and pharmaceutically acceptable salts, acids or derivatives of any of the agents listed herein.

In embodiments, the one or more chemotherapeutic agents are selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted urea compounds such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that can be administered in conjunction with a compound of the disclosure or composition thereof in embodiments of the present methods include fluorouracil (5-FU), alone or in further combination with leukovorin; other pyrimidine analogs such as UFT, capecitabine, gemcitabine and cytarabine, the alkyl sulfonates, e.g., busulfan, improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil, cyclophosphamide, estramustine, ifosfamide, novembrichin, prednimustine and uracil mustard; and triazines, e.g., dacarbazine. In embodiments, the alkylating agent is thiotepa or CYTOXAN cyclosphosphamide.

Examples of antimetabolite chemotherapeutic agents that can be administered in conjunction with a compound of the disclosure or composition thereof in embodiments of the present methods include folic acid analogs, e.g., methotrexate and pteropterin; and the purine analogs, such as mercaptopurine and thioguanine.

Examples of natural product-based chemotherapeutic agents that may be administered in conjunction with a compound of the disclosure or composition thereof in certain embodiments of the present method include the vinca alkaloids, e.g., vinblastine, vincristine and vindesine; the epipodophyllotoxins, e.g., etoposide and teniposide; the antibiotic chemotherapeutic agents, e.g., doxorubicin, epirubicin, mitomycin, dactinomycin, temozolomide, plicamycin, bleomycin; and the enzymatic chemotherapeutic agents such as L-asparaginase.

Examples of useful COX-II inhibitors that can be administered in conjunction with a compound of the disclosure or composition thereof in embodiments of the present method include Vioxx, CELEBREX (celecoxib), valdecoxib, paracoxib, rofecoxib, and Cox 189.

Examples of useful matrix metalloproteinase inhibitors that can be administered in conjunction with a compound of the disclosure or composition thereof in embodiments of the present method are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), WO 1999/007675, European Patent Application No. 99302232.1 (filed Mar. 25, 1999), Great Britain patent application number 9912961.1 (filed Jun. 3, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference for their teachings regarding the same. Preferred MMP-2 and MMP-9 inhibitors are those that have little or no activity inhibiting MMP-1. More preferred are those that selectively inhibit MMP-2 and/or MMP-9 relative to the other matrix-metalloproteinases (e.g., MMP-1, MMP-3, MMP-4, MMP-5, MMP-6, MMP-7, MMP-8, MMP-10, MMP-11, MMP-12, and MMP-13).

Some specific examples of MMP inhibitors are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R)-3-[4-(4-chloro-phenoxy)-benzenesulfonylamino]-tetrahydro-pyran-3-carboxylic acid hydroxyamide; (2R,3R)-1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Signal transduction inhibitors can also be administered in conjunction with a compound of the disclosure or composition thereof in embodiments of the present method. Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example, WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the methods described herein.

EGFR-inhibiting agents include the monoclonal antibodies C225 and anti-EGFR 22Mab (ImClone Systems, Inc., New York, N.Y.), the compounds ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used as additional therapeutic agents in embodiments of the present disclosure. VEGF inhibitors, for example, SU-5416 and SU-6668 (Sugen Inc., South San Francisco, Calif.), can also be combined with a compound of the disclosure or composition thereof in embodiments of the present methods. VEGF inhibitors are described in, for example, WO 01/60814 A3 (published Aug. 23, 2001), WO 99/24440 (published May 20, 1999), PCT International Application PCT/IB99/00797 (filed May 3, 1999), WO 95/21613 (published Aug. 17, 1995), WO 99/61422 (published Dec. 2, 1999), U.S. Pat. No. 5,834,504 (issued Nov. 10, 1998), WO 01/60814, WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference for their teachings regarding the same. Other examples of specific VEGF inhibitors useful in the present disclosure are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present disclosure as described herein. pErbB2 receptor inhibitors, such as GW-282974 (Glaxo Wellcome plc), and the monoclonal antibodies, AR-209 (Aronex Pharmaceuticals Inc., The Woodlands, Tex.) and 2B-1 (Chiron), can furthermore be combined with a compound of the disclosure or composition thereof in embodiments of the present methods. Further examples of pErbB2 receptor inhibitors and/or monoclonal antibodies include, for example, those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference for their teachings regarding the same. ErbB2 receptor inhibitors useful in the methods of this disclosure are also described in U.S. Pat. No. 6,284,764 (issued Sep. 4, 2001), incorporated in its entirety herein by reference for its teaching regarding the same. The erbB2 receptor inhibitor compounds and substance described in the aforementioned PCT applications and U.S. patents, as well as other compounds and substances that inhibit the erbB2 receptor, can be used in conjunction with a compound of the disclosure or composition thereof in embodiments of the methods of the present disclosure.

In various embodiments, methods of the disclosure further comprise administering to the mammal an effective amount of a pro-apoptotic agent and/or an agent that operates via apoptosis and/or an agent that operates via apoptosis driven by direct protein modulation. Examples of such agents include ABT-263 (Navitoclax), and obatoclax, WEP, bortezomib, and carfilzomib.

In any of the above embodiments, an effective amount of one or more of the following may also be administered to the mammal: (i) a bromodomain inhibitor (e.g., a Brd2 inhibitor, a Brd3 inhibitor, a Brd4 inhibitor and/or a BrdT inhibitor); (ii) a histone methyltransferase inhibitor (e.g., a DOT1-like histone methyltransferase (Dot1L) inhibitor); (iii) a histone deacetylase (HDAC) inhibitor (e.g., a Class I HDAC (e.g., HDAC1, HDAC2, HDAC3 and HDAC8) inhibitor, a Class IIa HDAC (e.g., HDAC4, HDAC5, HDAC7, and HDAC9) inhibitor; a Class IIb HDAC (e.g., HDAC6 and HDAC10) inhibitor; and a Class IV HDAC (e.g., HDAC11) inhibitor); and (iv) a histone demethylase inhibitor (e.g. an inhibitor of a lysine-specific demethylase, such as lysine-specific demethylase 1A (Lsd1)).

In embodiments, an effective amount of one or more therapeutic agents that inhibit bromodomain proteins such as Brd2, Brd3, Brd4 and/or BrdT, is also administered to a mammal. In some embodiments, the bromodomain inhibitor is a Brd4 inhibitor. In some embodiments, the bromodomain inhibitor is JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct. 10; 56(19):7498-500; GlaxoSmithKline), IBET151 (Bioorg. Med. Chem. Lett. 2012 Apr. 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium), or CPI-0610 (Constellation Pharmaceuticals).

In yet further embodiments, an effective amount of one or more therapeutic agents that inhibit histone methyltransferase proteins such as the DOT1-like histone methyltransferase (Dot1L) is also administered to a mammal. In some embodiments, the histone methyltransferase inhibitor is EPZ004777, EPZ-5676 (Blood. 2013 Aug. 8; 122(6):1017-25) or SGC0946 (Nat. Commun. 2012; 3:1288). In specific embodiments, the histone methyltransferase inhibitor is EPZ-5676.

In embodiments, an effective amount of one or more therapeutic agents that inhibit histone deacetylase (HDAC) proteins is also administered to the mammal. HDAC proteins may be grouped into classes based on homology to yeast HDAC proteins with Class I made up of HDAC1, HDAC2, HDAC3 and HDAC 8; Class IIa made up of HDAC4, HDAC5, HDAC7 and HDAC 9; Class IIb made up of HDAC6 and HDAC10; and Class IV made up of HDAC11. In some embodiments, the HDAC inhibitor is trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct. 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr. 13; 96(8):4592-7), mocetinostat (Bioorg. Med. Chem. Lett. 2008 Feb. 1; 18(3):1067-71) or valproic acid (EMBO J. 2001 Dec. 17; 20(24):6969-78). For example, in some embodiments, the HDAC inhibitor is panobinostat. In some embodiments, the HDAC inhibitor is panobinostat or SAHA.

In some embodiments, an effective amount of one or more therapeutic agents that inhibit histone demethylases, for example, lysine-specific demethylases, such as the lysine-specific demethylase 1A (Lsd1) is also administered to the mammal. In some embodiments, the histone demethylase inhibitor is HCl-2509 (BMC Cancer. 2014 Oct. 9; 14:752), tranylcypromine, or ORY-1001 (J. Clin. Oncol 31, 2013 (suppl; abstr e13543).

In embodiments, methods of the disclosure further comprise administering to the mammal an effective amount of a Brd4 inhibitor, a DNA methyltransferase inhibitor, or both. In some embodiments, the method further comprises administering a DNA methyltransferase inhibitor. In some more specific embodiments, the DNA methyltransferase inhibitor is azacitidine or decitabine. In certain related embodiments, the DNA methyltransferase inhibitor is azacitidine. In some other embodiments, the DNA methyltransferase inhibitor is decitabine. In still other embodiments, the DNA methyltransferase inhibitor is azacitidine and decitabine.

In embodiments, an effective amount of an intercalation agent is also administered to the mammal. In some such embodiments, the intercalation agent is mitoxanthrone. In embodiments, an effective amount of a Bcl-2 inhibitor is also administered to the mammal. In some such embodiments, the Bcl-2 inhibitor is venetoclax.

In embodiments, an effective amount of an fms-like tyrosine kinase 3 (FLT3) inhibitor is also administered to the mammal. In some embodiments, the FLT3 inhibitor is midostaurin. In embodiments, an effective amount of an Isocitrate dehydrogenase (IDH)1 or IDH2 inhibitor is also administered to the mammal. In some embodiments, the IDH1 or IDH2 inhibitor is gemtuzumab ozogamicin, enasidenib, or a combination thereof.

In some embodiments, an effective amount of an immunotherapy is also administered to the mammal. In some embodiments, an effective amount of a tyrosine kinase inhibitor is also administered to the mammal. In some embodiments, an effective amount of an immune checkpoint inhibitor (e.g., PD-L1-, PD-1-, CTLA-4-, LAG-3- or Tim-3-targeted agents) is also administered to the mammal.

A wide variety of cancers, including solid tumors and leukemias (e.g., acute myeloid leukemia and chronic lymphocytic leukemia) are amenable to the methods disclosed herein. Types of cancer that may be treated in various embodiments include, but are not limited to, adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell); histiocytic disorders; leukemia; histiocytosis malignant; Hodgkin's disease; immunoproliferative small; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor; adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma non-chromaffin; angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

In a still further aspect, the cancer is selected from cancers of the brain, genitourinary tract, endocrine system, gastrointestinal tract, blood, rectum, kidney, lymphatic system, stomach, and skin.

In some embodiments, the cancer is selected from cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cancer in adolescents, cervical cancer, childhood cancers, chordoma, cardiac tumors, chronic myeloproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal stromal tumors (GIST), gastrointestinal carcinoid tumor, germ cell tumor, gestational trophoblastic tumor, hairy cell leukemia, head and neck cancer, heart cancer, liver cancer, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, such as myelofibrosis, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, Viral-Induced cancer, acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, choroid plexus carcinoma, choroid plexus papilloma, craniopharyngioma, dysembryoplastic neuroepithelial tumor, gangliocytoma, germinoma, hemangioblastoma, hemangiopercytoma, metastatic brain tumor cell, and glioma (e.g., glioblastoma multiforme, ependymoma, astrocytoma, oligodendroglioma, oligoastrocytoma, juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, paraganglioma, or ganglioglioma cell).

In some embodiments, compound of the disclosure or compositions thereof (e.g., compounds of or compositions comprising a compound of structure (I)) are effective over a wide dosage range. For example, in the treatment of adult humans, dosages from 0.01 to 1000 mg, from 0.5 to 100 mg, from 1 to 50 mg, and from 5 to 40 mg per day are examples of dosages that are used in some embodiments. Other dosages that are used in some embodiments, for example, in the treatment of adult humans, include dosages from 0.1 to 100 mg, from 0.5 to 50 mg, from 0.5 to 10 mg and from 1 to 10 mg per day. An exemplary dosage is 10 to 30 mg per day. The exact dosage will depend upon the route of administration, the form in which the compound is administered, the subject to be treated, the body weight of the subject to be treated, and the preference and experience of the attending physician.

In some embodiments, the compound or composition is administered in a single dose. A single dose may also be used for treatment of an acute condition.

In some embodiments, a compound of the disclosure or composition thereof (e.g., a compound of or composition comprising a compound of structure (I)) is administered in multiple doses. In some embodiments, dosing is about once, twice, three times, four times, five times, six times, or more than six times per day. In other embodiments, dosing is about once a month, once every two weeks, once a week, or once every other day. In another embodiment a compound of the disclosure or composition thereof (e.g., a compound of or composition comprising a compound of structure (I)) and another agent(s) are administered together about once per day to about 6 times per day. In another embodiment, the administration of a compound of the disclosure (e.g., a compound of or composition comprising a compound of structure (I)) and another agent(s) continues for less than about 7 days. In yet another embodiment, the administration continues for more than about 6, about 10, about 14, about 28 days, about two months, about six months, or about one year. In some cases, continuous dosing is achieved and maintained as long as necessary.

Administration of the compound of the disclosure or composition thereof (e.g., the compound of or composition comprising a compound of structure (I)) may continue as long as necessary. In some embodiments, a compound of the disclosure or composition thereof (e.g., a compound of or composition comprising a compound of structure (I)) is administered for more than 1, 2, 3, 4, 5, 6, 7, 14, or 28 days. In some embodiments, a compound of the disclosure or composition thereof (e.g., a compound of or composition comprising a compound of structure (I)) is administered for less than 28, 14, 7, 6, 5, 4, 3, 2, or 1 day. In some embodiments, a compound of the disclosure or composition thereof (e.g., a compound of or composition comprising a compound of structure (I)) is administered chronically on an ongoing basis, e.g., for the treatment of chronic effects.

In some embodiments, the compound of the disclosure or composition thereof (e.g., the compound of or composition comprising a compound of structure (I)) are administered in dosages. Due to intersubject variability in compound pharmacokinetics, individualization of dosing regimen is provided in certain embodiments. Dosing for a compound of the disclosure or composition thereof (e.g., a compound of or composition comprising a compound of structure (I)) may be found by routine experimentation in light of the instant disclosure and/or can be derived by one of ordinary skill in the art.

More specifically, the therapeutic agents (e.g., a compound of or composition comprising a compound of structure (I), a tricyclic dione, and/or a nucleoside analog) described herein are effective over a wide dosage range. For example, in the treatment of adult humans, dosages from about 0.01 mg to about 1000 mg, from about 0.5 mg to about 100 mg, from about 1 mg to about 50 mg, and from about 5 mg to about 40 mg per day are examples of dosages that are used in some embodiments. An exemplary dosage is about 10 mg to about 30 mg per day.

In embodiments, a therapeutic agent is administered in a dose ranging from about 10 mg/m² to about 150 mg/m² per day. In embodiments, a therapeutic agent is administered in a dose ranging from about 50 mg/m² to about 130 mg/m² per day. In some embodiments, a therapeutic agent is administered in a dose ranging from about 30 mg/m² to about 50 mg/m² per day. In some embodiments, a therapeutic agent is administered in a dose ranging from about 35 mg/m² to about 45 mg/m² per day. In some embodiments, a therapeutic agent is administered in a dose ranging from about 550 mg/m² to about 750 mg/m² per day. In some embodiments, a therapeutic agent is administered in a dose ranging from about 650 mg/m² to about 675 mg/m² per day.

In certain embodiments, the dose of the compound of the disclosure or composition thereof (e.g., the compound of or composition comprising a compound of structure (I)) ranges from about 30 mg/m² to about 120 mg/m² per day. In some such embodiments, the dose of the compound of the disclosure or composition thereof (e.g., the compound of or composition comprising a compound of structure (I)) is administered via two or more routes of administration. In certain embodiments, the dose of the tricyclic dione ranges from about 25 mg/m² to about 55 mg/m² per day. In certain embodiments, the dose of the nucleoside analog (e.g., pyrimidine analog) ranges from about 600 mg/m² to about 700 mg/m² per day.

The exact dosage will depend upon the therapeutic agent, the route of administration, the form in which the compound is administered, the subject to be treated, physical and physiological factors including target, body weight, severity of condition, type of cancer, previous or concurrent therapeutic interventions, idiopathy of the subject, and the preference and experience of the attending physician.

In some embodiments, an effective amount of a therapeutic agent is administered in a single dose. Typically, such administration will be by injection, e.g., intravenous injection, in order to introduce the agent quickly. However, other routes are used as appropriate. A single dose of a compound of the disclosure may also be used for treatment of an acute condition.

In embodiments, a therapeutic agent is administered via a bolus injection in a dose ranging from about 15 mg/m² to about 35 mg/m² per day. In some embodiments, a therapeutic agent is administered via a bolus injection in a dose ranging from about 20 mg/m² to about 30 mg/m² per day. In some embodiments, a therapeutic agent is administered via intravenous infusion in a dose ranging from about 30 mg/m² to about 60 mg/m² per day. In embodiments, a therapeutic agent is administered via bolus injection in a dose ranging from about 45 mg/m² to about 90 mg/m² per day. In embodiments, a therapeutic agent is administered via intravenous infusion in a dose ranging from about 90 mg/m² to about 110 mg/m² per day.

In some embodiments, a compound of the disclosure or composition thereof (e.g., a compound of or composition comprising a compound of structure (I)) is administered via a bolus injection in a dose ranging from about 20 mg/m² to about 40 mg/m² per day. In some embodiments, a compound of the disclosure or composition thereof (e.g., a compound of or composition comprising a compound of structure (I)) is administered via intravenous infusion in a dose ranging from about 40 mg/m² to about 80 mg/m² per day. In embodiments, a tricyclic dione is administered via intravenous infusion in a dose ranging from about 30 mg/m² to about 50 mg/m² per day. In embodiments, a nucleoside analog (e.g., pyrimidine analog) is administered via intravenous infusion in a dose ranging from about 600 mg/m² to about 700 mg/m² per day.

In certain embodiments, the mammal has previously received one or more treatment regimens comprising a therapeutic agent for treatment of the disease.

In embodiments, methods of the present disclosure include administering the therapeutic agents described herein to the mammal based on mitochondrial integrity and/or MCL-1 dependency data obtained by contacting a mammal's cancer cell with a profiling peptide. In certain embodiments, the mammal has an MCL-1 dependency percentage of at least 15%, the MCL-1 dependency percentage having been obtained by an in vitro method comprising contacting a first portion of a plurality of cancer cells with a profiling peptide comprising a cellular uptake moiety and an MCL-1 binding domain, the MCL-1 binding domain having the sequence of SEQ ID NO:1 with 0-8 modifications.

Generally, profiling peptides comprise an Mcl-1 binding domain having a sequence shown in Table 6, which may be optionally modified.

TABLE 6
Exemplary Mcl-1 Binding Domains.

| SEQ ID | |
|---|---|
| NO: | Sequence |
| 1 | RPEIWMTQGLRRLGDEINAYYAR |
| 2 | RPEIWLTQSLQRLGDEINAYYAR |
| 3 | RPEIWLTQHLQRLGDEINAYYAR |
| 4 | RPEIWMGQGLRRLGDEINAYYAR |
| 5 | RPEIWLGQSLQRLGDEINAYYAR |
| 6 | RPEIWLGQHLQRLGDEINAYYAR |
| 7 | RPEIWITQELRRIGDEFNAYYAR |
| 8 | RPEIWMTQELRRIGDEFNAYYAR |
| 9 | RPEIWITQGLRRIGDEFNAYYAR |
| 10 | RPEIWITQELRRLGDEFNAYYAR |
| 11 | RPEIWITQELRRIGDEINAYYAR |

In some embodiments, a profiling peptide comprises an Mcl-1 binding domain having the sequence of any one of SEQ ID NOS:1-11 with 0-8 modifications. In some embodiments, a profiling peptide comprises an Mcl-1 binding domain having the sequence of any one of SEQ ID NOS:1-11 with 1-8 modifications.

“Modified” peptides include peptides having one or more amino acid substitutions as compared to a sequence disclosed herein. The substitution can be a conservative or a non-conservative substitution. Modified peptides also include peptides having additions of amino acids to, or deletions of amino acids from, the original peptide sequence. Therefore, modified peptides include fragments of the original peptide sequence. In some embodiments, each modification independently comprises a conservative amino acid substitution, an addition, or a deletion.

As used herein, a “modification” refers to a substitution, addition, or deletion of a single amino acid. Accordingly, when a number of modifications is referenced (e.g., an Mcl-1 binding domain having the sequence of SEQ ID NO:1 with two modifications), the number refers to the number of amino acids of the sequence that may be substituted, added, or deleted. In other words, each “substitution,” “addition,” or “deletion” replaces, adds, or removes a single amino acid, respectively, and does not refer to a single instance that replaces, adds, or removes more than one amino acid.

Modifications may be introduced by altering a polynucleotide encoding a profiling peptide, and may be performed by a variety of methods, including site-specific or site-directed mutagenesis. For example, mutations may be introduced at a particular location by synthesizing oligonucleotides containing a mutant sequence flanked by restriction sites enabling ligation to fragments of the unmodified sequence. Following ligation, the resulting sequence would encode a modified peptide having the desired amino acid addition, substitution, or deletion.

A “conservative substitution” includes a substitution found in one of the following conservative substitutions groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function or chemical structure or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Proline (Pro or P), and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

In embodiments, the modifications described herein may include the substitution of a naturally-occurring amino acid with a synthetic amino acid, amino acid analog, or amino acid mimetic, or the addition of a synthetic amino acid, amino acid analog, or amino acid mimetic. In such embodiments, modifications can include the substitution of one more L-amino acids with D-amino acids. The D-amino acid can be the same amino acid type as that found in the natural sequence or can be a different amino acid.

“Modification” also includes the substitution of a naturally-occurring amino acid with an amino acid that has been conjugated to, or otherwise associated with, a functional group. Such an amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. The presence of such amino acids may be preferred to, for example, increase polypeptide storage stability, and/or increase peptide solubility. Such modifications can be performed co-translationally or post-translationally during recombinant production, or by synthetic means.

In embodiments, the profiling peptides described herein comprise an Mcl-1 binding domain of any one of SEQ ID NOS:1-11 (e.g., SEQ ID NO:1) with 0 to 1 modifications. In some embodiments, the profiling peptides described herein comprise an Mcl-1 binding domain of any one of SEQ ID NOS:1-11 (e.g., SEQ ID NO:1) with 0 to 2 modifications. In some embodiments, the profiling peptides described herein comprise an Mcl-1 binding domain of any one of SEQ ID NOS:1-11 (e.g., SEQ ID NO:1) with 0 to 3 modifications. In some embodiments, the profiling peptides described herein comprise an Mcl-1 binding domain of any one of SEQ ID NOS:1-11 (e.g., SEQ ID NO:1) with 0 to 4 modifications. In some embodiments, the profiling peptides described herein comprise an Mcl-1 binding domain of any one of SEQ ID NOS:1-11 (e.g., SEQ ID NO:1) with 0 to 5 modifications. In some embodiments, the profiling peptides described herein comprise an Mcl-1 binding domain of any one of SEQ ID NOS:1-11 (e.g., SEQ ID NO:1) with 0 to 6 modifications. In some embodiments, the profiling peptides described herein comprise an Mcl-1 binding domain of any one of SEQ ID NOS:1-11 (e.g., SEQ ID NO:1) with 0 to 7 modifications. In some embodiments, the profiling peptides described herein comprise an Mcl-1 binding domain of any one of SEQ ID NOS:1-11 (e.g., SEQ ID NO:1) with 0 to 8 modifications. In some embodiments, the profiling peptides described herein comprise an Mcl-1 binding domain of any one of SEQ ID NOS:1-11 (e.g., SEQ ID NO:1) with 0 to 9 modifications, 0 to 10 modifications, 0 to 12 modifications, 0 to 15 modifications, or 0 to 20 modifications.

In some embodiments, the profiling peptides described herein comprise an Mcl-1 binding domain of any one of SEQ ID NOS:1-11 (e.g., SEQ ID NO:1) with 1 to 2 modifications. In some embodiments, the profiling peptides described herein comprise an Mcl-1 binding domain of any one of SEQ ID NOS:1-11 (e.g., SEQ ID NO:1) with 1 to 3 modifications. In some embodiments, the profiling peptides described herein comprise an Mcl-1 binding domain of any one of SEQ ID NOS:1-11 (e.g., SEQ ID NO:1) with 1 to 4 modifications. In some embodiments, the profiling peptides described herein comprise an Mcl-1 binding domain of any one of SEQ ID NOS:1-11 (e.g., SEQ ID NO:1) with 1 to 5 modifications. In some embodiments, the profiling peptides described herein comprise an Mcl-1 binding domain of any one of SEQ ID NOS:1-11 (e.g., SEQ ID NO:1) with 1 to 6 modifications. In some embodiments, the profiling peptides described herein comprise an Mcl-1 binding domain of any one of SEQ ID NOS:1-11 (e.g., SEQ ID NO:1) with 1 to 7 modifications. In some embodiments, the profiling peptides described herein comprise an Mcl-1 binding domain of any one of SEQ ID NOS:1-11 (e.g., SEQ ID NO:1) with 1 to 8 modifications. In some embodiments, the profiling peptides described herein comprise an Mcl-1 binding domain of any one of SEQ ID NOS:1-11 (e.g., SEQ ID NO:1) with 1 to 9 modifications. In some embodiments, the profiling peptides described herein comprise an Mcl-1 binding domain of any one of SEQ ID NOS:1-11 (e.g., SEQ ID NO:1) with 1 to 10 modifications, 1 to 12 modifications, 1 to 15 modifications, or 1 to 20 modifications.

In some embodiments, the profiling peptides described herein comprise an Mcl-1 binding domain of any one of SEQ ID NOS:1-11 (e.g., SEQ ID NO:1) with 2 to 3 modifications. In some embodiments, the profiling peptides described herein comprise an Mcl-1 binding domain of any one of SEQ ID NOS:1-11 (e.g., SEQ ID NO:1) with 2 to 4 modifications. In some embodiments, the profiling peptides described herein comprise an Mcl-1 binding domain of any one of SEQ ID NOS:1-11 (e.g., SEQ ID NO:1) with 2 to 5 modifications. In some embodiments, the profiling peptides described herein comprise an Mcl-1 binding domain of any one of SEQ ID NOS:1-11 (e.g., SEQ ID NO:1) with 2 to 6 modifications. In some embodiments, the profiling peptides described herein comprise an Mcl-1 binding domain of any one of SEQ ID NOS:1-11 (e.g., SEQ ID NO:1) with 2 to 7 modifications. In some embodiments, the profiling peptides described herein comprise an Mcl-1 binding domain of any one of SEQ ID NOS:1-11 (e.g., SEQ ID NO:1) with 2 to 8 modifications. In some embodiments, the profiling peptides described herein comprise an Mcl-1 binding domain of any one of SEQ ID NOS:1-11 (e.g., SEQ ID NO:1) with 2 to 9 modifications, 2 to 10 modifications, 2 to 12 modifications, 2 to 15 modifications, or 2 to 20 modifications.

In some embodiments, the profiling peptides described herein comprise an Mcl-1 binding domain of any one of SEQ ID NOS:1-11 (e.g., SEQ ID NO:1) with 3 to 4 modifications. In some embodiments, the profiling peptides described herein comprise an Mcl-1 binding domain of any one of SEQ ID NOS:1-11 (e.g., SEQ ID NO:1) with 3 to 5 modifications. In some embodiments, the profiling peptides described herein comprise an Mcl-1 binding domain of any one of SEQ ID NOS:1-11 (e.g., SEQ ID NO:1) with 3 to 6 modifications. In some embodiments, the profiling peptides described herein comprise an Mcl-1 binding domain of any one of SEQ ID NOS:1-11 (e.g., SEQ ID NO:1) with 3 to 7 modifications. In some embodiments, the profiling peptides described herein comprise an Mcl-1 binding domain of any one of SEQ ID NOS:1-11 (e.g., SEQ ID NO:1) with 3 to 8 modifications. In some embodiments, the profiling peptides described herein comprise an Mcl-1 binding domain of any one of SEQ ID NOS:1-11 (e.g., SEQ ID NO:1) with 3 to 9 modifications, 3 to 10 modifications, 3 to 12 modifications, 3 to 15 modifications, 3 to 20 modifications, 4 to 5 modifications, 4 to 6 modifications, 4 to 7 modifications, 4 to 8 modifications, 4 to 9 modifications, 4 to 10 modifications, 4 to 12 modifications, 4 to 15 modifications, 4 to 20 modifications, 5 to 6 modifications, 5 to 7 modifications, 5 to 8 modifications, 5 to 9 modifications, 5 to 10 modifications, 5 to 12 modifications, 5 to 15 modifications, 5 to 20 modifications, 6 to 7 modifications, 6 to 8 modifications, 6 to 9 modifications, 6 to 10 modifications, 7 to 8 modifications, 7 to 9 modifications, 7 to 10 modifications, 8 to 9 modifications, 8 to 10 modifications, or 9 to 10 modifications. In some embodiments, the profiling peptides described herein comprise a modified Mcl-1 binding domain of any one of SEQ ID NOS:1-11 (e.g., SEQ ID NO:1) with 1, 2, 3, 4, 5, 6, 7, 8, 9, 10, 11, 12, 13, 14, 15, 16, 17, 18, 19, or 20 modifications. The Mcl-1 binding domain sequence included in a profiling peptide of the present disclosure may include a modification at any position.

In embodiments where the Mcl-1 binding domain is a fragment of any one of SEQ ID NOS:1-11, the amino acid sequence can have a minimum length of 5 amino acids, 6 amino acids, 7 amino acids, 8 amino acids, 9 amino acids, 10 amino acids, 11 amino acids, 12 amino acids, 13 amino acids, or 14 amino acids. In some embodiments where the Mcl-1 binding domain is a fragment of any one of SEQ ID NOS:1-11, the amino acid sequence can have a minimum length of 15 amino acids. In some embodiments where the Mcl-1 binding domain is a fragment of any one of SEQ ID NOS:1-11, the amino acid sequence can have a minimum length of 16 amino acids. In some embodiments where the Mcl-1 binding domain is a fragment of any one of SEQ ID NOS:1-11, the amino acid sequence can have a minimum length of 17 amino acids. In some embodiments where the Mcl-1 binding domain is a fragment of any one of SEQ ID NOS:1-11, the amino acid sequence can have a minimum length of 18 amino acids. In some embodiments where the Mcl-1 binding domain is a fragment of any one of SEQ ID NOS:1-11, the amino acid sequence can have a minimum length of 19 amino acids. In some embodiments where the Mcl-1 binding domain is a fragment of any one of SEQ ID NOS:1-11, the amino acid sequence can have a minimum length of 20 amino acids. In some embodiments where the Mcl-1 binding domain is a fragment of any one of SEQ ID NOS:1-11, the amino acid sequence can have a minimum length of 21 amino acids.

In some embodiments, the Mcl-1 binding domain is a fragment of SEQ ID NO:1, and the amino acid sequence has a minimum length of 5 amino acids, 6 amino acids, 7 amino acids, 8 amino acids, 9 amino acids, 10 amino acids, 11 amino acids, 12 amino acids, 13 amino acids, or 14 amino acids. In some embodiments, the Mcl-1 binding domain is a fragment of SEQ ID NO:1, and the amino acid sequence has a minimum length of 15 amino acids. In some embodiments, the Mcl-1 binding domain is a fragment of SEQ ID NO:1, and the amino acid sequence has a minimum length of 16 amino acids. In some embodiments, the Mcl-1 binding domain is a fragment of SEQ ID NO:1, and the amino acid sequence has a minimum length of 17 amino acids. In some embodiments, the Mcl-1 binding domain is a fragment of SEQ ID NO:1, and the amino acid sequence has a minimum length of 18 amino acids. In some embodiments, the Mcl-1 binding domain is a fragment of SEQ ID NO:1, and the amino acid sequence has a minimum length of 19 amino acids. In some embodiments, the Mcl-1 binding domain is a fragment of SEQ ID NO:1, and the amino acid sequence has a minimum length of 20 amino acids. In some embodiments, the Mcl-1 binding domain is a fragment of SEQ ID NO:1, and the amino acid sequence has a minimum length of 21 amino acids.

In further embodiments, the Mcl-1 binding domain comprises at least 10 contiguous amino acids of any one of SEQ ID NOS:1-11 (e.g., SEQ ID NO:1). In some embodiments, the Mcl-1 binding domain comprises at least 11 contiguous amino acids of any one of SEQ ID NOS:1-11. In some embodiments, the Mcl-1 binding domain comprises at least 12 contiguous amino acids of any one of SEQ ID NOS:1-11. In some embodiments, the Mcl-1 binding domain comprises at least 13 contiguous amino acids of any one of SEQ ID NOS:1-11. In some embodiments, the Mcl-1 binding domain comprises at least 14 contiguous amino acids of any one of SEQ ID NOS:1-11. In some embodiments, the Mcl-1 binding domain comprises at least 15 contiguous amino acids of any one of SEQ ID NOS:1-11 (e.g., SEQ ID NO:1). In some embodiments, the Mcl-1 binding domain comprises at least 16 contiguous amino acids of any one of SEQ ID NOS:1-11. In some embodiments, the Mcl-1 binding domain comprises at least 17 contiguous amino acids of any one of SEQ ID NOS:1-11. In some embodiments, the Mcl-1 binding domain comprises at least 18 contiguous amino acids of any one of SEQ ID NOS:1-11. In some embodiments, the Mcl-1 binding domain comprises at least 19 contiguous amino acids of any one of SEQ ID NOS:1-11. In some embodiments, the Mcl-1 binding domain comprises at least 20 contiguous amino acids of any one of SEQ ID NOS:1-11 (e.g., SEQ ID NO:1). In some embodiments, the Mcl-1 binding domain comprises at least 21 contiguous amino acids of any one of SEQ ID NOS:1-11. In some embodiments, the Mcl-1 binding domain comprises at least at least 10 contiguous amino acids of SEQ ID NO:1, at least 11 contiguous amino acids of SEQ ID NO:1, at least 12 contiguous amino acids of SEQ ID NO:1, at least 13 contiguous amino acids of SEQ ID NO:1, at least 14 contiguous amino acids of SEQ ID NO:1, at least 15 contiguous amino acids of SEQ ID NO:1, at least 16 contiguous amino acids of SEQ ID NO:1, at least 17 contiguous amino acids of SEQ ID NO:1, at least 18 contiguous amino acids of SEQ ID NO:1, at least 19 contiguous amino acids of SEQ ID NO:1, at least 20 contiguous amino acids of SEQ ID NO:1, or at least 21 contiguous amino acids of SEQ ID NO:1.

In some embodiments, the Mcl-1 binding domain comprises no more than 10 contiguous amino acids of any one of SEQ ID NOS:1-11, no more than 11 contiguous amino acids of any one of SEQ ID NOS:1-11, no more than 12 contiguous amino acids of any one of SEQ ID NOS:1-11, no more than 13 contiguous amino acids of any one of SEQ ID NOS:1-11, no more than 14 contiguous amino acids of any one of SEQ ID NOS:1-11, no more than 15 contiguous amino acids of any one of SEQ ID NOS:1-11, no more than 16 contiguous amino acids of any one of SEQ ID NOS:1-11, no more than 17 contiguous amino acids of any one of SEQ ID NOS:1-11, no more than 18 contiguous amino acids of any one of SEQ ID NOS:1-11, no more than 19 contiguous amino acids of any one of SEQ ID NOS:1-11, no more than 20 contiguous amino acids of any one of SEQ ID NOS:1-11, or no more than 21 contiguous amino acids of any one of SEQ ID NOS:1-11. In some embodiments, the Mcl-1 binding domain comprises no more than 10 contiguous amino acids of SEQ ID NO:1, no more than 11 contiguous amino acids of SEQ ID NO:1, no more than 12 contiguous amino acids of SEQ ID NO:1, no more than 13 contiguous amino acids of SEQ ID NO:1, no more than 14 contiguous amino acids of SEQ ID NO:1, no more than 15 contiguous amino acids of SEQ ID NO:1, no more than 16 contiguous amino acids of SEQ ID NO:1, no more than 17 contiguous amino acids of SEQ ID NO:1, no more than 18 contiguous amino acids of SEQ ID NO:1, no more than 19 contiguous amino acids of SEQ ID NO:1, no more than 20 contiguous amino acids of SEQ ID NO:1, or no more than 21 contiguous amino acids of SEQ ID NO:1.

Embodiments of the Mcl-1 binding domains disclosed herein include amino acid sequences with at least 70% sequence identity to the sequence of any one of SEQ ID NOS:1-11. In some embodiments, the Mcl-1 binding domain has at least 75% sequence identity with the sequence of any one of SEQ ID NOS:1-11. In some embodiments, the Mcl-1 binding domain has at least 80% sequence identity with the sequence of any one of SEQ ID NOS:1-11. In some embodiments, the Mcl-1 binding domain has at least 85% sequence identity with the sequence of any one of SEQ ID NOS:1-11. In some embodiments, the Mcl-1 binding domain has at least 90% sequence identity with the sequence of any one of SEQ ID NOS:1-11. In some embodiments, the Mcl-1 binding domain has at least 95% sequence identity with the sequence of any one of SEQ ID NOS:1-11. In some embodiments, the Mcl-1 binding domain has at least 96% sequence identity with the sequence of any one of SEQ ID NOS:1-11. In some embodiments, the Mcl-1 binding domain has at least 97% sequence identity with the sequence of any one of SEQ ID NOS:1-11. In some embodiments, the Mcl-1 binding domain has at least 98% sequence identity with the sequence of any one of SEQ ID NOS:1-11. In some embodiments, the Mcl-1 binding domain has at least 99% sequence identity with the sequence of any one of SEQ ID NOS:1-11.

In some embodiments, the Mcl-1 binding domain has a sequence with at least 70% sequence identity to the sequence of SEQ ID NO:1. In some embodiments, the Mcl-1 binding domain has a sequence with at least 75% sequence identity to the sequence of SEQ ID NO:1. In some embodiments, the Mcl-1 binding domain has a sequence with at least 80% sequence identity to the sequence of SEQ ID NO:1. In some embodiments, the Mcl-1 binding domain has a sequence with at least 85% sequence identity to the sequence of SEQ ID NO:1. In some embodiments, the Mcl-1 binding domain has a sequence with at least 90% sequence identity to the sequence of SEQ ID NO:1. In some embodiments, the Mcl-1 binding domain has a sequence with at least 95% sequence identity to the sequence of SEQ ID NO:1. In some embodiments, the Mcl-1 binding domain has a sequence with at least 96% sequence identity to the sequence of SEQ ID NO:1. In some embodiments, the Mcl-1 binding domain has a sequence with at least 97% sequence identity to the sequence of SEQ ID NO:1. In some embodiments, the Mcl-1 binding domain has a sequence with at least 98% sequence identity to the sequence of SEQ ID NO:1. In some embodiments, the Mcl-1 binding domain has a sequence with at least 99% sequence identity to the sequence of SEQ ID NO:1.

“Percent sequence identity” refers to a relationship between two or more sequences, as determined by comparing the sequences. Preferred methods to determine sequence identity are designed to give the best match between the sequences tested. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. In embodiments, the length of a sequence aligned for comparison purposes is at least 70%, 80%, 90%, or 100% of the length of the reference sequence. In embodiments, the percent sequence identity referenced herein is calculated over the length of the reference sequence. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure it will be understood that where sequence analysis software is used for analysis, the results of the analysis are based on the “default values” of the program referenced. “Default values” mean any set of values or parameters which originally load with the software when first initialized.

In embodiments, a modified Mcl-1 binding domain retains the specificity and affinity for binding to Mcl-1 of the unmodified sequence (i.e., the modifications to the Mcl-1 binding domain do not alter the specificity or affinity for binding to Mcl-1 in a statistically significant, clinically significant, or biologically significant manner). In some embodiments, a modified Mcl-1 binding domain retains the specificity and affinity for binding to Mcl-1 of the unmodified sequence if the specificity and affinity of the modified Mcl-1 binding domain are at least 70%, 80%, 85%, 90%, 95%, 97%, or 99% of the specificity and affinity of the unmodified sequence. For example, a modified Mcl-1 binding domain may retain the specificity and affinity for binding to Mcl-1 of the unmodified sequence if the specificity and affinity of the modified Mcl-1 binding domain are at least at least 70%, 80%, 85%, 90%, 95%, 97%, or 99% of the specificity and affinity of any one of SEQ ID NOS:1-11.

In embodiments, the Mcl-1 binding domain binds to Mcl-1 with at least a 20-fold increased affinity over NOXA. In some embodiments, the Mcl-1 binding domain binds to Mcl-1 with at least a 22-fold increased affinity over NOXA. In some embodiments, the Mcl-1 binding domain binds to Mcl-1 with at least a 24-fold increased affinity over NOXA. In particular embodiments, the Mcl-1 binding domain binds to Mcl-1 with at least a 28-fold increased affinity over NOXA.

The effect of any amino acid modification to an Mcl-1 binding domain may be determined empirically by testing the resulting modified Mcl-1 binding domain for the ability to function in a biological assay, or to bind to a target molecule, such as a monoclonal or polyclonal antibody. For example, the ability of the modified Mcl-1 binding domain to fold into a conformation comparable to the unmodified sequence can be tested using assays known in the art, including reacting with monoclonal or polyclonal antibodies that are specific for the native or unfolded peptides, testing the retention of binding functions, and testing the sensitivity or resistance of the modified Mcl-1 binding domain to digestion with proteases.

Analysis and/or computer modeling of the primary and secondary amino acid structure of the Mcl-1 binding domain to analyze the tertiary structure of the peptide may aid in identifying specific amino acid residues that can be substituted, added, or deleted without significantly altering the structure and, as a consequence, potentially significantly reducing the binding specificity and affinity of the Mcl-1 binding domain.

In embodiments, the Mcl-1 binding domain has a sequence of any one of SEQ ID NOS:1-11. In some embodiments, the Mcl-1 binding domain has a sequence of SEQ ID NO:1.

In embodiments, profiling peptides of the present disclosure further comprise a cellular uptake moiety, which is optionally joined to the Mcl-1 binding domain by a linker. A “cellular uptake moiety” refers to an amino acid sequence or chemical compound that, when conjugated to a peptide, allows the peptide and the cellular uptake moiety to cross the outer cell membrane, thereby transferring the peptide into the cell. Additionally, in some embodiments, the cellular uptake moiety may act as a targeting moiety, such that it directs the peptide to a desired cellular location (e.g., the mitochondria).

In embodiments, the cellular uptake moiety is a peptide sequence. In such embodiments, the cellular uptake moiety peptide is at least four amino acids in length, at least five amino acids in length, at least six amino acids in length, at least seven amino acids in length, at least eight amino acids in length, or at least nine amino acids in length. In some embodiments, the cellular uptake moiety comprises an amino acid sequence of 1 to 20 amino acids, 5 to 20 amino acids, 6 to 20 amino acids, 7 to 20 amino acids, 8 to 20 amino acids, 9 to 20 amino acids, 10 to 20 amino acids, 11 to 20 amino acids, 12 to 20 amino acids, 15 to 20 amino acids, 1 to 15 amino acids, 5 to 15 amino acids, 6 to 15 amino acids, 7 to 15 amino acids, 8 to 15 amino acids, 9 to 15 amino acids, 10 to 15 amino acids, 11 to 15 amino acids, 12 to 15 amino acids, 1 to 12 amino acids, 5 to 12 amino acids, 6 to 12 amino acids, 7 to 12 amino acids, 8 to 12 amino acids, 9 to 12 amino acids, 10 to 12 amino acids, 1 to 10 amino acids, 5 to 10 amino acids, 6 to 10 amino acids, or 7 to 10 amino acids.

In embodiments, the cellular uptake moiety peptide is a transduction domain isolated from a known peptide sequence. Peptides with transduction domains are well known in the art and include, for example, human immunodeficiency virus (HIV) Trans-Activator of Transcription (TAT; described in Vives et al., J Biol Chem. 1997 Jun. 20; 272(25):16010-7), Herpes simplex virus tegument protein VP22, Atennapedia plasma membrane (ANT) translocation domain, a poly-Arg sequence, and the like. In embodiments, the cellular uptake moiety peptide is a continuous amino acid sequence from a known transduction domain. In other embodiments, the cellular uptake moiety peptide is two or more amino acid sequences from one or more known transduction domains that are not naturally present in a contiguous amino acid sequence, for example, a cellular uptake domain comprising two amino acid sequences would be separated by a third amino acid sequence in nature.

In embodiments, the cellular uptake moiety peptide is an optionally modified transduction domain from a known peptide. The modifications may be made using known techniques.

In embodiments, the cellular uptake moiety peptide is an optionally modified TAT translocation domain. The optionally modified TAT translocation domain can have 0 to 1 modifications, 0 to 2 modifications, 0 to 3 modifications, 0 to 4 modifications, 0 to 5 modifications, 0 to 6 modifications, 0 to 7 modifications, 0 to 8 modifications, 0 to 9 modifications, 1 to 2 modifications, 1 to 3 modifications, 1 to 4 modifications, 1 to 5 modifications, 1 to 6 modifications, 1 to 7 modifications, 1 to 8 modifications, 1 to 9 modifications, 2 to 3 modifications, 2 to 4 modifications, 2 to 5 modifications, 2 to 6 modifications, 2 to 7 modifications, 2 to 8 modifications, 2 to 9 modifications, 3 to 4 modifications, 3 to 5 modifications, 3 to 6 modifications, 3 to 7 modifications, 3 to 8 modifications, 3 to 9 modifications, 4 to 5 modifications, 4 to 6 modifications, 4 to 7 modifications, 4 to 8 modifications, or 4 to 9 modifications. In embodiments where the cellular uptake moiety peptide is a fragment of the TAT translocation domain, the cellular uptake moiety peptide sequence can have a minimum length of 5 amino acids, 6 amino acids, 7 amino acids, 8 amino acids, 9 amino acids, or 10 amino acids. In some embodiments where the cellular uptake moiety peptide is a fragment of the TAT translocation domain, the cellular uptake moiety peptide sequence can have a minimum of 5 contiguous amino acids of a TAT translocation domain. In some embodiments, the cellular uptake moiety peptide sequence can have a minimum of 5 contiguous amino acids, 6 contiguous amino acids, 7 contiguous amino acids, 8 contiguous amino acids, 9 contiguous amino acids, or 10 contiguous amino acids of a TAT translocation domain. Modified TAT translocation domains disclosed herein include amino acid sequences with at least 70% sequence identity, at least 75% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to the sequence of YGRKKRRQRRR (SEQ ID NO:12). In some embodiments, the cellular uptake moiety has at least 90% identity with SEQ ID NO:12. In some embodiments, the cellular uptake moiety is a modified TAT translocation domain. In other embodiments, the cellular uptake moiety is a TAT translocation domain comprising the sequence of SEQ ID NO:12.

In embodiments, the profiling peptide of the present disclosure comprises a TAT translocation domain and an Mcl-1 binding domain having a sequence of SEQ ID NOS:1-11 with 0-8 modifications. In some embodiments, the profiling peptide of the present disclosure comprises a TAT translocation domain and an Mcl-1 binding domain having a sequence of SEQ ID NOS:1-11 with 1-8 modifications. In embodiments, the profiling peptide of the present disclosure comprises a TAT translocation domain and an Mcl-1 binding domain having SEQ ID NO:1 with 0-8 modifications. In some embodiments, the profiling peptide of the present disclosure comprises a TAT translocation domain and an Mcl-1 binding domain having a sequence of any one of SEQ ID NOS:1-11. In some embodiments, the profiling peptide of the present disclosure comprises a TAT translocation domain having the sequence of SEQ ID NO:12 and an Mcl-1 binding domain having a sequence of any one of SEQ ID NOS:1-11 with 0-8 modifications. In some embodiments, the profiling peptide of the present disclosure comprises a TAT translocation domain having the sequence of SEQ ID NO:12 and an Mcl-1 binding domain having a sequence of any one of SEQ ID NOS:1-11 with 1-8 modifications. In embodiments, the profiling peptide of the present disclosure comprises a TAT translocation domain having the sequence of SEQ ID NO:12 and an Mcl-1 binding domain having the sequence of SEQ ID NO:1 with 0-8 modifications. In some embodiments, the profiling peptide of the present disclosure comprises a TAT translocation domain having the sequence of SEQ ID NO:12 and an Mcl-1 binding domain having a sequence of any one of SEQ ID NOS:1-11.

In embodiments, the cellular uptake moiety peptide is an optionally modified ANT translocation domain. The optionally modified ANT translocation domain can have 0 to 1 modifications, 0 to 2 modifications, 0 to 3 modifications, 0 to 4 modifications, 0 to 5 modifications, 0 to 6 modifications, 0 to 7 modifications, 0 to 8 modifications, 0 to 9 modifications, 1 to 2 modifications, 1 to 3 modifications, 1 to 4 modifications, 1 to 5 modifications, 1 to 6 modifications, 1 to 7 modifications, 1 to 8 modifications, 1 to 9 modifications, 2 to 3 modifications, 2 to 4 modifications, 2 to 5 modifications, 2 to 6 modifications, 2 to 7 modifications, 2 to 8 modifications, 2 to 9 modifications, 3 to 4 modifications, 3 to 5 modifications, 3 to 6 modifications, 3 to 7 modifications, 3 to 8 modifications, 3 to 9 modifications, 4 to 5 modifications, 4 to 6 modifications, 4 to 7 modifications, 4 to 8 modifications, or 4 to 9 modifications. In embodiments where the cellular uptake moiety peptide is a fragment of the ANT translocation domain, the cellular uptake moiety peptide sequence can have a minimum length of 5 amino acids, 6 amino acids, 7 amino acids, 8 amino acids, 9 amino acids, or 10 amino acids. In some embodiments where the cellular uptake moiety peptide is a fragment of the ANT translocation domain, the cellular uptake moiety peptide sequence can have a minimum of 5 contiguous amino acids of an ANT translocation domain. In some embodiments, the cellular uptake moiety peptide sequence can have a minimum of 6 contiguous amino acids, 7 contiguous amino acids, 8 contiguous amino acids, 9 contiguous amino acids, or 10 contiguous amino acids of an ANT translocation domain. Modified ANT translocation domains disclosed herein include amino acid sequences with at least 70% sequence identity, at least 75% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to the sequence of RQIKIWFQNRRMKWKK (SEQ ID NO:13). In some embodiments, the cellular uptake moiety has at least 90% identity with SEQ ID NO:13. In some embodiments, the cellular uptake moiety peptide is a modified ANT translocation domain. In other embodiments, the cellular uptake moiety peptide comprises an ANT translocation domain having the sequence of SEQ ID NO:13.

In embodiments, the profiling peptide of the present disclosure comprises an ANT translocation domain and an Mcl-1 binding domain having a sequence of any one of SEQ ID NOS:1-11 with 0-8 modifications. In some embodiments, the profiling peptide of the present disclosure comprises an ANT translocation domain and an Mcl-1 binding domain having a sequence of any one of SEQ ID NOS:1-11 with 1-8 modifications. In embodiments, the profiling peptide of the present disclosure comprises an ANT translocation domain and an Mcl-1 binding domain having the sequence of SEQ ID NO:1 with 0-8 modifications. In embodiments, the profiling peptide of the present disclosure comprises an ANT translocation domain and an Mcl-1 binding domain having a sequence of any one of SEQ ID NOS:1-11. In some embodiments, the profiling peptide of the present disclosure comprises an ANT translocation domain having the sequence of SEQ ID NO:13 and an Mcl-1 binding domain having a sequence of any one of SEQ ID NOS:1-11 with 0-8 modifications. In some embodiments, the profiling peptide of the present disclosure comprises an ANT translocation domain having the sequence of SEQ ID NO:13 and an Mcl-1 binding domain having a sequence of any one of SEQ ID NOS:1-11 with 1-8 modifications. In embodiments, the profiling peptide of the present disclosure comprises an ANT translocation domain having the sequence of SEQ ID NO:13 and an Mcl-1 binding domain having the sequence of SEQ ID NO:1 with 0-8 modifications. In some embodiments, the profiling peptide of the present disclosure comprises an ANT translocation domain having the sequence of SEQ ID NO:13 and an Mcl-1 binding domain having a sequence of any one of SEQ ID NOS:1-11.

In embodiments, the cellular uptake moiety is an arginine rich amino acid sequence, such as a poly-Arg sequence. An amino acid sequence is “arginine rich” if greater than 50% of the amino acids of the cellular uptake moiety are arginine. In some embodiments, the arginine rich amino acid sequence includes 3 to 9 Arg residues, 3 to 10 Arg residues, 3 to 11 Arg residues, 3 to 12 Arg residues, 4 to 9 Arg residues, 4 to 10 Arg residues, 4 to 11 Arg residues, 4 to 12 Arg residues, 5 to 9 Arg residues, 5 to 10 Arg residues, 5 to 11 Arg residues, 5 to 12 Arg residues, 6 to 9 Arg residues, 6 to 10 Arg residues, 6 to 11 Arg residues, 6 to 12 Arg residues, 7 to 9 Arg residues, 7 to 10 Arg residues, 7 to 11 Arg residues, 7 to 12 Arg residues, 8 to 9 Arg residues, 8 to 10 Arg residues, 8 to 11 Arg residues, 8 to 12 Arg residues, 9 to 10 Arg residues, 9 to 11 Arg residues, or 9 to 12 Arg residues. In some embodiments, the poly-Arg sequence includes 3 Arg residues, 4 Arg residues, 5 Arg residues, 6 Arg residues, 7 Arg residues, 8 Arg residues, 9 Arg residues, 10 Arg residues, 11 Arg residues, or 12 Arg residues. In some embodiments, the poly-Arg sequence includes 2 to 10 contiguous Arg residues. In some embodiments, the poly-Arg sequence includes 3 to 9 contiguous Arg residues, 3 to 10 contiguous Arg residues, 3 to 11 contiguous Arg residues, 3 to 12 contiguous Arg residues, 4 to 9 contiguous Arg residues, 4 to 10 contiguous Arg residues, 4 to 11 contiguous Arg residues, 4 to 12 contiguous Arg residues, 5 to 9 contiguous Arg residues, 5 to 10 contiguous Arg residues, 5 to 11 contiguous Arg residues, 5 to 12 contiguous Arg residues, 6 to 9 contiguous Arg residues, 6 to 10 contiguous Arg residues, 6 to 11 contiguous Arg residues, 6 to 12 contiguous Arg residues, 7 to 9 contiguous Arg residues, 7 to 10 contiguous Arg residues, 7 to 11 contiguous Arg residues, 7 to 12 contiguous Arg residues, 8 to 9 contiguous Arg residues, 8 to 10 contiguous Arg residues, 8 to 11 contiguous Arg residues, 8 to 12 contiguous Arg residues, 9 to 10 contiguous Arg residues, 9 to 11 contiguous Arg residues, or 9 to 12 contiguous Arg residues.

In embodiments, the profiling peptide of the present disclosure comprises an arginine rich amino sequence and an Mcl-1 binding domain having a sequence of any one of SEQ ID NOS:1-11 with 0-8 modifications. In some embodiments, the profiling peptide of the present disclosure comprises an arginine rich amino sequence and an Mcl-1 binding domain having a sequence of any one of SEQ ID NOS:1-11 with 1-8 modifications. In embodiments, the profiling peptide of the present disclosure comprises an arginine rich amino acid sequence and an Mcl-1 binding domain having the sequence of SEQ ID NO:1 with 0-8 modifications. In some embodiments, the profiling peptide of the present disclosure comprises a poly-Arg sequence having 2 to 10 contiguous Arg residues and an Mcl-1 binding domain having a sequence of any one of SEQ ID NOS:1-11 with 0-8 modifications. In some embodiments, the profiling peptide of the present disclosure comprises a poly-Arg sequence having 2 to 10 contiguous Arg residues and an Mcl-1 binding domain having a sequence of any one of SEQ ID NOS:1-11 with 1-8 modifications.

In embodiments, the profiling peptide of the present disclosure comprises an arginine rich amino sequence and an Mcl-1 binding domain having a sequence of any one of SEQ ID NOS:1-11. In some embodiments, the profiling peptide of the present disclosure comprises a poly-Arg sequence having 3 to 10 contiguous Arg residues and an Mcl-1 binding domain having a sequence of any one of SEQ ID NOS:1-11 with 0-8 modifications. In some embodiments, the profiling peptide of the present disclosure comprises a poly-Arg sequence having 3 to 10 contiguous Arg residues and an Mcl-1 binding domain having a sequence of any one of SEQ ID NOS:1-11 with 1-8 modifications. In embodiments, the profiling peptide of the present disclosure comprises a poly-Arg sequence having 3 to 10 Arg residues and an Mcl-1 binding domain having the sequence of SEQ ID NO:1 with 0-8 modifications. In some embodiments, the profiling peptide of the present disclosure comprises a poly-Arg sequence having 3 to 10 contiguous Arg residues and an Mcl-1 binding domain having a sequence of any one of SEQ ID NOS:1-11.

In embodiments, a modified cellular uptake moiety peptide retains the ability of the unmodified sequence to cross the cell membrane when conjugated to a peptide (i.e., the modifications to the cellular uptake moiety peptide do not alter the ability to cross the cell membrane when conjugated to a peptide in a statistically significant, clinically significant, or biologically significant manner). In some embodiments, a modified cellular uptake moiety peptide retains the ability of the unmodified sequence to cross the cell membrane when conjugated to a peptide if the internalization efficiency of the modified cellular uptake moiety peptide is at least 70%, 80%, 85%, 90%, 95%, 97%, or 99% of the internalization efficiency of the unmodified sequence.

Alternatively, the cellular uptake moiety can be a chemical compound. Chemical compounds that facilitate cellular internalization are understood by one of skill in the art, and include, for example, cholesterol moieties, octanoic acid, lithocholic acid, oleyl alcohol, lithocholic acid oleylamide, and decanoic acid.

The Mcl-1 binding domain and the cellular uptake moiety can be linked by chemical coupling in any suitable manner known in the art. The cellular uptake moiety may be linked to the Mcl-1 binding domain at any suitable location. In some embodiments, the cellular uptake moiety is conjugated to the N-terminus of the Mcl-1 binding domain. In other embodiments, the cellular uptake moiety is conjugated to the C-terminus of the Mcl-1 binding domain.

In embodiments, the cellular uptake moiety is conjugated to the Mcl-1 binding domain via a linker. In further embodiments, the cellular uptake moiety is conjugated via a linker to the N-terminus of the Mcl-1 binding domain. In still further embodiments, the cellular uptake moiety is conjugated via a linker to the C-terminus of the Mcl-1 binding domain.

Suitable linkers include peptide sequences of any length and other chemical linkers as would be understood by one of ordinary skill. Short peptide sequences are employed in certain embodiments, for example peptide sequences including uncharged amino acids, non-polar amino acids and/or small amino acids. In some embodiments, a linker is an amino acid sequence of 1-5 amino acids. For example, some exemplary linkers include Gly, Pro, Ala, Val, Leu, Met, Ile, and/or Phe amino acids. Other examples of suitable peptide sequences include two Pro residues, three Gly residues, and the like. In some embodiments, the cellular uptake moiety is linked to the Mcl-1 binding domain in such a way that the cellular uptake moiety is cleaved upon or after entry into the cell. In certain embodiments, the linker comprises three Gly residues, for example GGG.

Embodiments of the profiling peptides of the present disclosure may be 20 to 40 amino acids in length, 20 to 45 amino acids in length, 20 to 50 amino acids in length, 25 to 40 amino acids in length, 25 to 45 amino acids in length, 25 to 50 amino acids in length, 30 to 36 amino acids in length, 30 to 37 amino acids in length, 30 to 38 amino acids in length, 30 to 39 amino acids in length, 30 to 40 amino acids in length, 30 to 45 amino acids in length, 30 to 50 amino acids in length, 31 to 36 amino acids in length, 31 to 37 amino acids in length, 31 to 38 amino acids in length, 31 to 39 amino acids in length, 31 to 40 amino acids in length, 32 to 36 amino acids in length, 32 to 37 amino acids in length, 32 to 38 amino acids in length, 32 to 39 amino acids in length, 32 to 40 amino acids in length, 33 to 36 amino acids in length, 33 to 37 amino acids in length, 33 to 38 amino acids in length, 33 to 39 amino acids in length, 33 to 40 amino acids in length, 34 to 36 amino acids in length, 34 to 37 amino acids in length, 34 to 38 amino acids in length, 34 to 39 amino acids in length, 34 to 40 amino acids in length, 35 to 36 amino acids in length, 35 to 37 amino acids in length, 35 to 38 amino acids in length, 35 to 39 amino acids in length, 35 to 40 amino acids in length, 35 to 45 amino acids in length, 35 to 50 amino acids in length, 36 to 37 amino acids in length, 36 to 38 amino acids in length, 36 to 39 amino acids in length, 36 to 40 amino acids in length, 37 to 38 amino acids in length, 37 to 39 amino acids in length, 37 to 40 amino acids in length, 38 to 39 amino acids in length, 38 to 40 amino acids in length, or 39 to 40 amino acids in length.

In embodiments, a profiling peptide comprises a cellular uptake moiety, and an Mcl-1 binding domain having a sequence of any one of SEQ ID NOS:1-11 with 0-8 modifications. In embodiments, a profiling peptide comprises a cellular uptake moiety, and an Mcl-1 binding domain having a sequence of any one of SEQ ID NOS:1-11 with 1-8 modifications. In embodiments, a profiling peptide comprises a cellular uptake moiety, and an Mcl-1 binding domain having the sequence of SEQ ID NO:1 with 0-8 modifications. In embodiments, a profiling peptide comprises a cellular uptake moiety, and an Mcl-1 binding domain having a sequence of any one of SEQ ID NOS:1-11. In some embodiments, a profiling peptide comprises a cellular uptake moiety, and an Mcl-1 binding domain having the sequence of SEQ ID NO:1.

In embodiments, a profiling peptide comprises a cellular uptake moiety having the sequence of SEQ ID NO:12 conjugated to an Mcl-1 binding domain having a sequence of any one of SEQ ID NOS:1-11 with 0-8 modifications by a linker. In embodiments, a profiling peptide comprises a cellular uptake moiety having the sequence of SEQ ID NO:12 conjugated to an Mcl-1 binding domain having a sequence of any one of SEQ ID NOS:1-11 with 1-8 modifications by a linker. In embodiments, a profiling peptide comprises a cellular uptake moiety of the sequence of SEQ ID NO:12 conjugated to an Mcl-1 binding domain having the sequence of SEQ ID NO:1 with 0-8 modifications by a linker. In embodiments, a profiling peptide comprises a cellular uptake moiety having the sequence of SEQ ID NO:12 conjugated to an Mcl-1 binding domain having a sequence of any one of SEQ ID NOS:1-11 by a linker.

In embodiments, the profiling peptide has the sequence of

(SEQ ID NO: 14)
YGRKKRRQRRRGGGRPEIWMTQGLRRLGDEINAYYAR
or
(SEQ ID NO: 15)
RPEIWMTQGLRRLGDEINAYYARGGGYGRKKRRQRRR.

In certain embodiments, the profiling peptide comprises the sequence of SEQ ID NO:14. In other embodiments, the profiling peptide comprises the sequence of SEQ ID NO:15. In specific embodiments, the profiling peptide consists of the sequence of SEQ ID NO:14. In other embodiments, the profiling peptide consists of the sequence of SEQ ID NO:15. In certain embodiments, the profiling peptide comprises the sequence of SEQ ID NO:14 or SEQ ID NO:15. In specific embodiments, the profiling peptide consists of the sequence of SEQ ID NO:14 or SEQ ID NO:15.

Modified profiling peptides may be synthesized and purified by standard chemical methods. Peptides may be chemically synthesized by manual techniques or by automated procedures. Equipment for automated synthesis of peptides is commercially available from suppliers such as Perkin-Elmer, Inc. (Waltham, Mass.) and may be operated according to the manufacturer's instructions. Additionally, synthesized profiling peptides may be obtained from any number of different custom peptide synthesizing manufacturers. If required, synthesized peptides may be purified using preparative reverse phase chromatography, partition chromatography, gel filtration, gel electrophoresis, ion-exchange chromatography, or other methods used in the art.

Alternatively, modified profiling peptides may be readily prepared by genetic engineering and recombinant molecular biology methods and techniques. For example, polynucleotides encoding modified profiling peptides, or fragments thereof, may be constructed by recombinant methods or chemically synthesized (using such devices as an automatic synthesizer). Methods for purifying polynucleotides after either chemical synthesis or recombinant synthesis are known to persons skilled in the art. The constructed or synthesized polynucleotides may be incorporated into expression vectors (e.g., a plasmid, a viral particle, or a phage) for production of the profiling peptide in a host cell into which the expression vector has been introduced. Polynucleotides that encode a profiling peptide described herein may be recombinantly expressed in a variety of different host cells. Host cells may then be genetically engineered (transduced, transformed, or transfected) with the expression vectors. Selection and maintenance of culture conditions for particular host cells, such as temperature, pH and the like, will be readily apparent to the ordinarily skilled artisan. The produced peptides may then be harvested and purified using methods known in the art.

Such peptides may be used to produce a sensitivity profile for a cancer cell or a plurality of cancer cells. In certain embodiments, the cancer cell or plurality of cancer cells is from a human tumor-derived cell line. In certain embodiments, the cancer cell or plurality of cancer cells is cancer stem cells. In some embodiments, the cancer cell or plurality of cancer cells is isolated from a tumor. In certain embodiments, the cancer cell or plurality of cancer cells is derived from the biopsy of a non-solid tumor. In embodiments, the cancer cell or plurality of cancer cells is obtained from peripheral blood from the subject. In other embodiments the cancer cell or plurality of cancer cells is obtained from bone marrow of the subject.

In embodiments, the cancer cells are from a solid cancer. In some embodiments, the cancer cell or plurality of cancer cells is derived from a solid tumor. In embodiments, the cancer cell or plurality of cancer cells is derived from the biopsy of a solid tumor, such as, for example, a biopsy of a colorectal, breast, prostate, lung, pancreatic, renal, or ovarian primary tumor. In some embodiments, the cancer cell or plurality of cancer cells is a circulating tumor cell. In embodiments, the cancer cells are from a non-solid cancer. In various embodiments, the cancer cells are from a pre-metastatic cancer. In various embodiments, the cancer cells are from a metastatic cancer.

In some embodiments, the cancer cell or plurality of cancer cells is derived from a a mammal with a hematologic cancer. In some embodiments, the cancer cell or plurality of cancer cells is derived from multiple myeloma, MDS, AML, ALL, acute lymphocytic leukemia, chronic lymphogenous leukemia, CLL, mantle cell lymphoma, diffuse large B-cell lymphoma, follicular lymphoma, or non-Hodgkin's lymphoma. In specific embodiments, the cancer cell or plurality of cancer cells is derived from the biopsy of a mammal with multiple myeloma, AML, acute lymphocytic leukemia, chronic lymphogenous leukemia, mantle cell lymphoma, diffuse large B-cell lymphoma, and non-Hodgkin's lymphoma. In certain embodiments, the cancer is AML.

In a specific embodiment, the cancer cell or plurality of cancer cells is a multiple myeloma cell that is enriched by selection from a biopsy sample with an anti-CD138 antibody bound to a solid matrix or bead. In a specific embodiment, the cancer cell or plurality of cancer cells is an AML cell that is enriched by binding to a CD45-directed antibody. In a specific embodiment, the cancer cell or plurality of cancer cells is from a chronic lymphogenous leukemia or diffuse large B-cell lymphoma that is enriched by non-B cell depletion.

In various embodiments, the plurality of cancer cells is from a sample that has been frozen. In such embodiments, the sample may be warmed using a quick thaw process. The sample may then be added to culture medium and incubated.

In other embodiments, the plurality of cancer cells is from a sample that has not been frozen, i.e., that has been freshly collected. In such embodiments, the sample is added to culture medium and incubated after being isolated.

In some embodiments, such methods include isolating a cancer cell or a plurality of cancer cells from a mammal. In embodiments, a plurality of cancer cells isolated from a mammal is separated into two or more portions. In embodiments, a plurality of cancer cells isolated from a mammal is separated into three or more portions. In some embodiments, multiple replicates are tested for each portion.

In embodiments, the cancer cell, the plurality of cancer cells, or a portion thereof, is contacted with one or more profiling peptides disclosed herein and a percent polarization is determined. In embodiments, the cancer cell, the plurality of cancer cells, or a portion thereof, is contacted with one or more profiling peptides disclosed herein and a change in mitochondrial integrity of the cell(s) is detected. In various embodiments, more than one profiling peptide may be used at once. In such embodiments, a panel of profiling peptides (e.g., 2, 3, 4, 5, 10, etc. profiling peptides) may be screened on a single mammal specimen.

In some embodiments, the cancer cell, the plurality of cancer cells, or a portion thereof, is contacted with a composition comprising a profiling peptide. In such embodiments, the composition may comprise a profiling peptide in a concentration ranging from about 1.5 μM to about 2.5 μM. In embodiments, the composition comprises a profiling peptide in a concentration ranging from about 1.75 μM to about 2.25 μM. In embodiments, the composition comprises a profiling peptide in a concentration of about 2.0 μM.

In embodiments, the plurality of cancer cells, or a portion thereof, is contacted with one or more profiling peptides for about 15 minutes to about 45 minutes. In some embodiments, the plurality of cancer cells, or a portion thereof, is contacted with one or more profiling peptides for about 20 minutes to about 40 minutes. In some embodiments, the plurality of cancer cells, or a portion thereof, is contacted with one or more profiling peptides for about 30 minutes.

A percent polarization can be related to a change in mitochondrial integrity in the cell or plurality of cells. A change in mitochondrial integrity can be detected in any suitable manner, such as, for example, a change in mitochondrial membrane potential, chromatin condensation, loss of viability, Cytochrome C translocation from the mitochondrial intermembrane space to the cytosol, swelling of the mitochondria, mitochondrial fission, morphological changes (e.g., cell shrinkage, membrane blebbing, etc.), phosphatidyl serine externalization (e.g., as measured by annexin V staining) or the increase in reactive oxygen intermediates. As is understood by one of skill in the art, various methods of detection for each of the indications of a change in mitochondrial integrity may be employed. In embodiments, the change in mitochondrial integrity will be a decrease in mitochondrial integrity. In some embodiments, the decrease in mitochondrial integrity is measured by a decrease in mitochondrial membrane potential. The decrease in mitochondrial potential may be determined using any suitable method known in the art. In some embodiments, the decrease in mitochondrial integrity is measured by Cytochrome C leakage. In some embodiments, the decrease will be a statistically significant, clinically significant, or biologically significant decrease. In some embodiments, the decrease is a 2%, 5%, 10%, 15%, 20%, 25%, 30%, 35%, 40%, 45%, 50%, 60%, 70%, 80%, or 90% difference in a measurement of mitochondrial integrity, as described herein, as compared to a control.

A change in mitochondrial membrane potential may be measured using any suitable detecting agent. The detecting agent can be any suitable agent, such as a fluorescent dye, a non-fluorescent dye that can be converted to a fluorescent dye, an antibody, and the like. Fluorescent dyes include, for example, 5,5′,6,6′-tetrachloro-1,1′,3,3′-tetraethyl-imidacarbocyanine iodide (JC-1), propidium iodide (PI), 1,1′,3,3,3′,3′-hexamethylindodicarbo-cyanine iodide (DilC1), and 3,3′-dihexyloxacarbocyanine iodide (DiOC6). In various embodiments, the fluorescent dye is a potentiometric dye. Suitable potentiometric dyes include, for example, DilC1, JC-1, and rhodamine 123. In embodiments, the potentiometric dye included is JC-1 or rhodamine 123. In other embodiments, the dye is dihydrorhodamine 123, a non-fluorescent dye that can be converted via oxidation to rhodamine 123, a fluorescent dye.

In embodiments, the plurality of cancer cells, or a portion thereof, is contacted with a dye in a concentration ranging from about 0.5 nM to about 1.5 nM. In embodiments, the plurality of cancer cells, or a portion thereof, is contacted with a dye in a concentration ranging from about 0.75 nM to about 1.25 nM. In embodiments, the plurality of cancer cells, or a portion thereof, is contacted with a dye in a concentration of about 1.0 μM. In embodiments, the plurality of cancer cells, or a portion thereof, is contacted with a dye for about 60 minutes to about 120 minutes. In embodiments, the plurality of cancer cells, or a portion thereof, is contacted with a dye for about 75 minutes to about 105 minutes. In embodiments, the plurality of cancer cells, or a portion thereof, is contacted with a dye for about 80 minutes to about 100 minutes. In embodiments, the plurality of cancer cells, or a portion thereof, is contacted with a dye for about 90 minutes.

In another example, Cytochrome C translocation can be measured using immunofluorescence staining. In a further example, an increase in reactive oxygen intermediates can be measured using flow cytometric analysis after staining with carboxy-dichlorofluorescein diacetate.

In various embodiments, the plurality of cancer cells is divided into two or more portions for the purposes of profiling. In embodiments, one portion is treated with a negative control and one portion is contacted with one or more profiling peptides or a composition comprising one or more profiling peptides disclosed herein. Any suitable negative control may be used. Examples of negative controls include water and water soluble organic solvents, such as DMSO, ethanol, and methanol. In some embodiments, the negative control is water.

Accordingly, methods of the present disclosure comprise contacting a first portion of a plurality of cancer cells with a profiling peptide comprising a cellular uptake moiety and an Mcl-1 binding domain having the sequence of SEQ ID NO:1 with 0-8 modifications; contacting a second portion of the plurality of cancer cells with a negative control; and determining a percent polarization of the first portion and the second portion of the plurality of cancer cells.

In some embodiments, one portion of the plurality of cancer cells is contacted with a positive control. In such embodiments, methods of the disclosure further comprise contacting a third portion of the plurality of cancer cells with a positive control. Any suitable positive control may be used. Examples of positive controls include carbonyl cyanide-4-(trifluoromethoxy)phenylhydrazone (FCCP), carbonyl cyanide m-chlorophenyl hydrazone (CCCP), N5,N6-bis(2-fluorophenyl)-[1,2,5]oxadiazolo[3,4-b]pyrazine-5,6-diamine (BAM-15), and the like. In particular embodiments, the positive control used is CCCP.

In embodiments, the plurality of cancer cells, or a portion thereof, is contacted with a positive control in a concentration ranging from about 25 μM to about 250 μM. In embodiments, the plurality of cancer cells, or a portion thereof, is contacted with a positive control in a concentration ranging from about 25 μM to about 200 μM. In embodiments, the plurality of cancer cells, or a portion thereof, is contacted with a positive control in a concentration ranging from about 50 μM to about 150 μM. In embodiments, the plurality of cancer cells, or a portion thereof, is contacted with a positive control in a concentration of about 100 μM.

In embodiments, at least a portion of the plurality of cancer cells is contacted with a blocking buffer that blocks receptors on the cells that bind to the fragment crystallizable (FC) region of antibodies (i.e., FC receptors). In embodiments, the plurality of cancer cells is contacted with the blocking buffer before being separated into portions. In embodiments, the blocking buffer is incubated with the plurality of cancer cells at room temperature.

In some embodiments, a plurality of cancer cells is contacted with one or more labels. In embodiments, the plurality of cancer cells is contacted with a label before being separated into portions. In some embodiments that use flow cytometry, the labels are fluorophores attached to antibodies or a chemical entity with affinity for a cell membrane feature or other cellular structure. In other embodiments that use flow cytometry, the labels are quantum dots attached to antibodies or a chemical entity with affinity for a cell membrane feature or other cellular structure. In any of these embodiments, the antibodies or chemical entities may recognize any suitable cell surface marker, such as CD3, CD13, CD20, CD33, CD34, or CD45. In various embodiments, a combination of labels is used.

In embodiments, the label comprises at least one monoclonal antibody. In some embodiments, the at least one monoclonal antibody comprises an anti-CD45 antibody, an anti-CD3 antibody, an anti-CD20 antibody, an anti-CD13 antibody, an anti-CD33 antibody, an anti-CD34 antibody, an anti-CD117 antibody, an anti-HLA-DR antibody, or a combination thereof.

In some embodiments, an additive with a high affinity for calcium channels is added to the plurality of cancer cells, or a portion thereof. In some such embodiments, the additive is a diterpenoid. In particular embodiments, the additive is ryanodine. In embodiments, the additive is added in a concentration that is sufficient to significantly reduce or prevent nonspecific dye uptake. In some embodiments, the additive is added in a concentration of at least about 20 μM. In some embodiments, the additive is added in a concentration of at least about 30 μM. In embodiments, the diterpenoid is added in a concentration ranging from about 10 μM to about 50 μM. In embodiments, the diterpenoid is added in a concentration ranging from about 20 μM to about 40 μM. In embodiments, the diterpenoid is added in a concentration of about 30 μM.

In some embodiments, an ATP synthase inhibitor is added to the plurality of cancer cells, or a portion thereof. In some such embodiments, the ATP synthase inhibitor is an oligomycin. In particular embodiments, the oligomycin is oligomycin A. In some embodiments, the ATP synthase inhibitor is added in a concentration of at least 0.25 μM. In some embodiments, the ATP synthase inhibitor is added in a concentration of at least about 0.5 μM. In some embodiments, the ATP synthase inhibitor is added in a concentration of no more than about 1.0 μM. In embodiments, the ATP synthase inhibitor is added in a concentration ranging from about 0.25 μM to about 0.75 μM. In embodiments, the ATP synthase inhibitor is added in a concentration ranging from about 0.4 μM to about 0.6 μM. In embodiments, the ATP synthase inhibitor is added in a concentration of about 0.5 μM.

In embodiments, the plurality of cancer cells is then contacted with a detecting agent, as described above. In some embodiments, the detecting agent is a dye. In some embodiments, the detecting agent is a fluorescent dye. In some embodiments, the detecting agent is a potentiometric dye. In certain embodiments, the dye is JC-1, DiOC₆, or rhodamine 123. In certain embodiments, the dye is JC-1 or rhodamine 123. In particular embodiments, the dye is DiOC₆.

In some embodiments, the plurality of cancer cells, or a portion thereof, is washed prior to being contacted with the detecting agent.

In such embodiments, the plurality of cancer cells may then be analyzed using flow cytometry. In embodiments, determining the percent polarization of a first portion and a second portion of the plurality of cancer cells comprises analyzing the first portion and the second portion of the plurality of cancer cells by flow cytometry. In some embodiments, a third portion of the plurality of cancer cells is also analyzed by flow cytometry.

Any suitable gating may be used in flow cytometry analysis. In embodiments, analyzing the first portion, second portion, and third portion of the plurality of cancer cells by flow cytometry comprises gating on the positive control. In some embodiments, such gating is on the CD45 dim, CD13, CD33, and CD34 high population of blast cells. In other embodiments, such gating is the CD34 dim, CD3 and CD20 high population. Accordingly, embodiments of the present disclosure include a method of producing a sensitivity profile for a plurality of cancer cells from a subject, the method comprising: isolating the plurality of cancer cells from a sample, contacting the plurality of cancer cells with a label, treating a first portion of the plurality of cancer cells with a negative control, treating a second portion of the plurality of cancer cells with a positive control, treating a third portion of the plurality of cancer cells with a profiling peptide comprising a cellular uptake moiety and an Mcl-1 binding domain having the sequence of SEQ ID NO:1 with 0-8 modifications, or a composition comprising a profiling peptide comprising a cellular uptake moiety and an Mcl-1 binding domain having the sequence of SEQ ID NO:1 with 0-8 modifications and a carrier, contacting the first portion, the second portion, and the third portion of the plurality of cancer cells with a dye and analyzing the first portion, the second portion, and the third portion of the plurality of cancer cells by flow cytometry.

Some methods described herein further comprise determining an Mcl-1 dependency percentage for the first portion of the plurality of cancer cells based at least on the percent polarization of the first portion and the second portion of the plurality of cancer cells.

In embodiments, polarized and depolarized cells are counted. The percent polarization can then be calculated by dividing the number of polarized cells by the total number of cells and multiplying by 100. In such embodiments, blasts are gated as described above. The blast gate in then plotted on a histogram for detection agent signal. A gate is then created specifically on the polarized cells, and the percent of cells which respond to treatment with a profiling peptide can be found with the following equation:

$$Percent Priming = ( \frac{N C - P P}{N C} ) \times 100 \qquad ( 1 )$$

NC is the average percent polarization of the second portion of the plurality of cancer cells and PP is the percent polarization of the first portion of the plurality of cancer cells.

In embodiments, the Mcl-1 dependency percentage (MDP) is defined by the following equation:

MDP=C×Avg[Percent Priming] (2)

where C is a calibration factor and Avg[Percent Priming] is the average of the percent priming for one or more replicates.

In embodiments, the calibration factor ranges from about 0.1 to about 3.0. In some embodiments, the calibration factor ranges from about 0.5 to about 2.5. In some embodiments, the calibration factor ranges from about 1.0 to about 2.0. In some embodiments, the calibration factor ranges from about 1.4 to about 1.8. In some embodiments, the calibration factor is about 1.5.

Unless otherwise noted, the Mcl-1 dependency percentages calculated herein correspond to a profiling peptide concentration of 1 μM with CCCP as the positive control and water or DMSO as the negative control. In some embodiments, the time occurs over a window from between about 0 to about 300 minutes, or about 0 to about 30 minutes.

For example, in some embodiments, the mammal has an MCL-1 dependency percentage of at least 20%. In other related embodiments, the mammal has an MCL-1 dependency percentage of at least 30%. In more specific embodiments, the mammal has an MCL-1 dependency percentage of at least 40%.

In an illustrative method of the disclosure, a plurality of cancer cells prepared, and sample quality is confirmed. In the case of a frozen sample, samples are quickly thawed, for example, by placing them in a 37° C. water bath for about 60-70 seconds. After thawing the sample, the cells are transferred to a flask containing warm culture medium. After incubation, the quality (e.g., viability, cell count, etc.) is confirmed. In the case of a fresh sample, mononuclear cells are isolated from bone marrow aspirates following standard laboratory protocol (e.g., Ficoll-Paque separation). The cells are counted and the viability of the isolated cells is determined. The cells are then transferred to a flask containing warm culture medium. The cells are then pelleted and re-suspended in a buffer that blocks FC receptors. A mix of monoclonal antibodies is then added and incubated. After incubating with the label, the cells are again pelleted. The cells are again re-suspended in a mix of an assay buffer that has a pH ranging from about 7.4 to about 7.6, a diterpenoid (e.g., ryanodine), and an ATP synthase inhibitor (e.g., Oligomycin A). The suspended cells are then aliquoted into three portions. The first portion is mixed with a negative control (e.g., nuclease free water), the second portion is mixed with a positive control (e.g., CCCP), and the third portion is mixed with the profiling peptide (e.g., having the sequence of SEQ ID NO:14). The three portions are then incubated. After the incubation, the cells are pelleted and re-suspended in the assay buffer. A dye (e.g., DiOC₆) is then added to each portion of cells.

In the illustrative method, the cells are analyzed via flow cytometry. The control cell lines are analyzed using the live gate. The sample is analyzed by gating single, live cells by plotting forward versus side scatter. Outliers containing high forward and side scatter values may be assumed to be doublets and dying cells, respectively, and may be excluded from the final analysis. Events that are very low in both forward and side scatter may also be excluded as cellular debris. Generate a dot plot of channel FL5 (PC7 labeled anti-CD45 antibody) against side scatter and identify the CD45 dim population. Using only the events within the “CD45 dim” gate, generate a dot plot of channel FL4 (PC5 labeled anti-CD13, anti-CD33 and anti-CD34 antibodies) against channel FL3 (ECD/PE-Texas Red labeled anti-CD3 and anti-CD20 antibodies). In order to gate exclusively on AML blasts, gate cells that are high in channel FL4 and low in channel FL3 (live cell population). Using only events within the “Blasts” population, generate a histogram of channel FL1. Determine the cells high in channel FL1. Apply the gates created to all aliquots of all treatments of the same sample.

In other embodiments, the MDP is defined by the following equation:

$$M D P = [ 1 - ( \frac{P e p - P C}{N C - P C} ) ] * 100 \qquad ( 3 )$$

Where PC is the area under the curve (“AUC”) of the positive control, NC is the AUC of the negative control, and Pep is the AUC of the profiling peptide. Unless otherwise noted, the MCL-1 dependency percentages calculated herein correspond to a profiling peptide concentration of 1 μM with CCCP as the positive control and water or DMSO as the negative control. The AUC is either area under the curve or signal intensity. In embodiments, the AUC is the median fluorescent intensity (MFI). In some embodiments, the area under the curve is established by homogenous time-resolved fluorescence (HTRF). In some embodiments, the time occurs over a window from between about 0 to about 300 minutes, or about 0 to about 30 minutes. In some embodiments, the area under the curve is established by fluorescence activated cell sorting (FACS) or microplate assay as known in the art or described herein. In some embodiments, the signal intensity is a single time point measurement that occurs between about 5 minutes and about 300 minutes.

In an illustrative method of the disclosure, a plurality of cancer cells are isolated from a mammal sample, and sample quality is confirmed. The cells are then pelleted, blocked in BSA, and labeled. After staining, cells are pelleted and separated into three portions and treated with water or dimethyl sulfoxide (DMSO) (negative control), CCCP (positive control) or a profiling peptide of the disclosure (subject dependency). DiOC6, a cationic mitochondrial dye is added. Later, the cells are analyzed via flow cytometry.

In some embodiments, a plurality of cancer cells are isolated from primary bone marrow aspirates and sample quality is determined. Cells are then pelleted, blocked in BSA and labeled for markers specific to B- and T-cells, as well as monocyte differentiation markers and blast-specific markers. After staining, cells are pelleted and separated into three portions and treated with either water (negative control), CCCP (positive control) or SEQ ID NO:14 (subject dependency). DiOC6, a cationic mitochondrial dye is added. The cells are analyzed via flow cytometry. Blast cells are isolated by gating on the CD45 dim, CD13, CD33, and CD34 high population of each sample.

In particular embodiments, a plurality of cancer cells are isolated from primary bone marrow aspirates using density-gradient centrifugation. Sample quality is determined using trypan blue exclusion. Cells are then pelleted, blocked in BSA and labeled for markers specific to B- and T-cells, as well as monocyte differentiation markers and blast-specific markers. After staining, cells are pelleted and separated into fluorescent-activated cell sorting (FACS) tubes and treated with either water (negative control), CCCP (positive control) or the sequence of SEQ ID NO:14 (subject dependency). DiOC6, a cationic mitochondrial dye is added. The cells are then analyzed via flow cytometry. Blast cells are isolated by gating on the CD45 dim, CD13, CD33 and CD34 high population of each sample.

In any of the above embodiments, the cancer cell, the plurality of cancer cells, or a portion thereof, is not permeabilized, for example with a cell permeabilization agent such as digitonin.

Embodiments of this disclosure are further illustrated by the following non-limiting examples.

### EXAMPLES

### Example 1

### Illustrative Assay Procedure

### Controls

A single set of controls (one positive and one negative) is included in every run. The controls are treated in the same manner as frozen samples.

### Samples

The assay may be performed on fresh or frozen BMMCs.

Frozen Sample
1. Prepare a T25 flask for each sample with 10 mL of RPMI 1640 culture medium warmed for 15 to 30 minutes in a 37° C. (5% CO₂) incubator. Add 150 U of DNase I to each flask.
2. Quickly thaw the samples (frozen BMMCs) by placing them in a 37° C. water bath for ˜60-70 seconds.
3. Immediately after thawing each sample, add the entire contents of the tube (should be at least 2 million cells with at least 40% viability) to one T25 flask containing warmed culture medium, dropwise and with a constant swirling motion of the medium.
4. Incubate in a 37° C. (5% CO₂) incubator for one hour.
5. After one hour of incubation, count the cells and ensure that the sample requirements (at least 2 million cells with at least 40% viability) are still met.

Fresh Sample
1. Isolate mononuclear cells from bone marrow aspirates following standard laboratory protocol. Count the cells and determine the viability of the isolated cells. If viability is >40%, seed 2 million BMMCs in cell culture medium using the procedure below.
2. Prepare a T25 flask with 10 mL of RPMI 1640 culture medium warmed for 15 to 30 minutes in a 37° C. (5% CO2) incubator.
3. Add 2 million of the freshly prepared cells to the T25 flask containing warmed culture medium, dropwise with a constant swirling motion of the medium.
4. Incubate in a 37° C. (5% CO₂) incubator for one hour.

### Cell Blocking and Staining

1. For each sample or control being run, transfer the cells from the T25 flask with RPMI medium into a 15 mL conical tube and spin the cells down at 350×g for 5 minutes at room temperature.
2. Decant the RPMI medium and resuspend the cells in 1 mL of room temperature Blocking Buffer. Pipette gently to mix.
3. Incubate the cells at room temperature for 15 minutes to allow for blocking of the FC receptors.
4. Add 15 μL of Monoclonal Antibody Mix to the cell suspension and incubate for 30 minutes at room temperature in the dark.

### Signal Stabilizer Treatment

Assay Buffer Master Mix Calculation

Volume of Assay Buffer (mL)=number of (samples+controls)×2 mL

Equation 1: Calculation of the Assay Buffer Volume (mL)

Volume of Signal Stabilizer (in μL)=Volume (in mL) of Assay Buffer needed×1.5

### Equation 2: Calculation of Stabilizer Volume (aL)

TABLE 7
i) Volume of Assay Buffer and Stabilizer

| Number of | Volume of Assay | Volume of |
|---|---|---|
| (Sample + Control) | Buffer (mL) | Stabilizer (μL) |
| 3 | 6 | 9 |
| 4 | 8 | 12 |

### Treatment Procedure

1. Upon completion of the Cell Blocking and Staining incubation, spin down the 15 mL conical tubes at 350×g for 5 minutes at room temperature.
2. For each sample and control, eight 12×75 mm flow tubes are used. Of those eight, 3 will be used for Nuclease-Free Water, 1 will be used for Depolarizing Solution, and 4 will be used for the profiling peptide.

TABLE 8
Number of Replicates.

| | Nuclease-Free | Depolarizing | SEQ ID NO: 14 |
|---|---|---|---|
| | Water (“W”) | Solution (“D”) | (“T”) |
| Number of | 3 | 1 | 4 |
| Replicates | | | |

3. Thaw one tube of Signal Stabilizer (containing ryanodine and oligomycin). Make sure it is completely thawed and well mixed before use.
4. Calculate the appropriate volume of Assay Buffer and Signal Stabilizer per the total number of samples and controls. Use Table 7 as a reference.
5. In a separate tube, prepare the Assay Buffer and Signal Stabilizer mix based on the calculations in step 4. Vortex to mix.
6. Decant the Blocking Buffer/Antibody Mix from the pelleted cells. Re-suspend each sample and control cell pellet in 2 mL of the master mix (prepared in step 5).
7. Lightly vortex each of the cell suspensions for a few seconds.
8. For each sample and control, aliquot 250 μL of the resultant cell suspension into each of the eight tubes from step 2.
9. Add 2.5 μL Nuclease-Free Water, or 2.5 μL Depolarizing Solution, or 2.5 μL SEQ ID NO:14 Peptide each to their respective tubes.
10. Lightly vortex the tubes for a few seconds.
11. Incubate all tubes at 37° C. (5% CO₂) for 30 minutes.

### Cell Washing and Mitochondrial Staining

1. Once the incubation is complete, spin the tubes of treated cells at 350×g for 5 minutes at room temperature. Decant the supernatant.
2. Re-suspend each cell pellet in 500 μL of room temperature Assay Buffer. Spin down the cells at 350×g for 5 minutes at room temperature.
3. Thaw one tube of Mitochondrial Staining Solution. Make sure it is completely thawed and well mixed before use.
4. During centrifugation, prepare the working dye solution in Assay Buffer by mixing 1 of Mitochondrial Staining Solution stock for every 1 mL of Assay Buffer needed. Vortex to mix.

TABLE 9
Volume for Assay Buffer and
Mitochondrial Staining Solution.

| | | Volume | Volume of |
|---|---|---|---|
| Number of | Volume of | of Working | Mitochondrial |
| (Samples + | Assay Buffer | Dye | Staining |
| Controls) | (mL) | Solution (mL) | Solution (μL) |
| 3 | 12 | 12 | 12 |
| 4 | 16 | 16 | 16 |

5. After the spin is complete, decant the Assay Buffer. Resuspend each cell pellet in 500 μL of the working dye solution.
6. Lightly vortex the tubes for a few seconds.
7. Incubate all tubes at 37° C. (5% CO₂) for 90 minutes.
8. Proceed promptly to Data Acquisition when the incubation is complete.

### Data Acquisition

Data can be acquired on a Beckman Coulter FC500 Flow Cytometer using a 488-nm laser. The wavelengths detected are listed in Table 10.

TABLE 10
List of detection wavelengths and
their common fluorophores.

| | Wavelength | Common Fluorophores/ |
|---|---|---|
| Channel | Detected | Channel Names |
| FL1 | 525 nm/40 | FITC |
| FL2 | 575 nm/40 | PE |
| FL3 | 620 nm/20 | ECD/PE-Texas Red |
| FL4 | 675 nm/40 | PC5 |
| FL5 | 755 nm/40 | PC7 |

### Compensation Acquisition

Perform the compensation according to the manufacturer's protocol.

### Sample Acquisition

Acquire data on the controls before the samples.
1. For each control and sample set, load the tubes onto the carousel in the following order:
   a. Water
   b. Depolarizing Solution
   c. SEQ ID NO:14 Peptide
2. Adjust the flow speed to “Low” and begin the run.
3. Set the forward and side scatter, as well as PMT voltages using the first Nuclease-Free Water tube according to standard Beckman Coulter FC500 procedures.
4. Once the voltages have been correctly set, increase the flow speed to “High”. Acquire 25,000 total events per replicate of each condition of each sample.
   a. PMT Voltage: Once the voltages have been set for the first Nuclease-Free Water condition, these voltages should not be changed for any of the following tubes of the same sample. The voltages may be changed between different samples but should remain the same for all runs of the same sample.
5. After sample acquisition is finished, dispose of all tubes according to local disposal regulations.

### Data Analysis

Sample Analysis
1. Gate single, live cells by plotting forward versus side scatter. Outliers containing high forward and side scatter values are assumed to be doublets and dying cells, respectively, and are excluded from the final analysis. Events that are very low in both forward and side scatter may also be excluded as cellular debris.
2. Label this gate “Live” and apply this gate to samples.
3. Select events included inside the “Live” gate.
4. Generate a dot plot of channel FL5 against side scatter and identify the CD45 dim population. Label this gate “CD45 dim.”
5. Using only the events within the “CD45 dim” gate, generate a dot plot of channel FL4 against channel FL3. In order to gate exclusively on AML blasts, gate cells that are high in channel FL4 and low in channel FL3. Label this gate “Blasts.”
6. Using only events within the “Blasts” population, generate a histogram of channel FL1. Determine the cells high in channel FL1 and label this gate “Polarized Cells.”
7. Apply the gates created to all aliquots of all treatments (W, D, and T) of the same sample.
8. Once the gates have been applied to all aliquots of a sample, conduct a visual inspection of each gate on each treatment to ensure that a slight difference in staining between each treatment has not due to a gate being misplaced.

Controls
1. Analyze the controls using only the “Live” gate. No other gating strategy is required for the controls because they are a pure population of one cell line.
2. Using only events within the “Live” population, generate a histogram of channel FL1. Determine the cells high in channel FL1 and label this gate “Polarized Cells.”
3. Once the “Polarized Cells” gate has been determined, analyze the control cell lines.

Percent Priming Calculation
1. The percent priming for each sample is calculated using the percentage of cells with the “Polarized Cells” gate.
2. Calculate the percent priming for each Peptide replicate individually using Equation 3; “Average % Polarized Water” is the average percent of cells present in the “Polarized Cells” gate of the three water replicates, and “% Polarized Peptide” is the percent of cells in the “Polarized Cells” gate of one of the Peptide replicates.

$$\begin{matrix}Equation for Calculation Percent Priming & \\ \% Priming = ( \frac{( Average \% Polarized Water - \% Polarized Peptide )}{Average \% Polarized Water} ) \times 100 & Equation 3\end{matrix}$$

3. The percent priming value should range from 0% to 100% for each of the replicates. Any value less than 0% will be adjusted to 0% and any value greater 100% will be adjusted to 100%.
4. The priming values for each of the replicates is then averaged and this value is multiplied by 1.6 (Equation 4).

Calibrated % Priming=Average % Priming×1.6

Equation 4: Equation for Calibration of the Average Percent Priming Value

The calibrated percent priming value should be below 100%. Any value greater 100% will be reported as 100%.

The teachings of all patents, published applications and references cited herein, including all of the U.S. patents, U.S. patent application publications, U.S. patent applications, foreign patents, foreign patent applications and non-patent publications referred to in this specification, including U.S. Provisional Patent Application Ser. No. 62/163,188, filed May 18, 2015, are incorporated herein by reference in their entirety to the extent not inconsistent with the present description.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

1. A compound having the following structure (I): 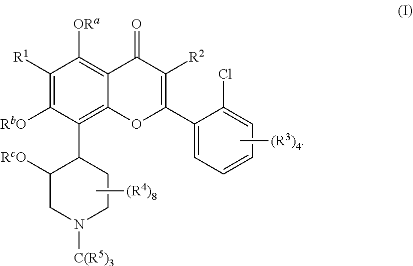
or a stereoisomer, tautomer or pharmaceutically acceptable salt thereof, wherein:
R^{a}, R^{b} and R^{c} are each independently H, D or —P(═O)(OR)₂;
R, R¹, R², R³, R⁴ and R⁵ are, at each occurrence, independently H or D;
wherein at least one occurrence of R, R^{a}, R^{b}, R^{c}, R¹, R², R³, R⁴ and R⁵ is D.

2. The compound of claim 1, wherein the compound has the following structure (I′):

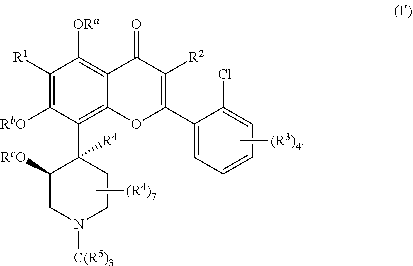

3. The compound of claim 1, wherein the compound has the following structure (I″):

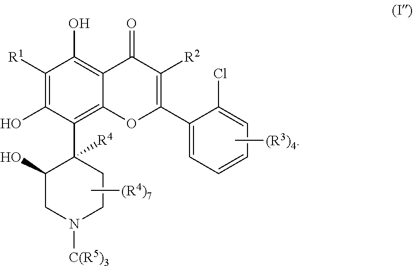

4. The compound of claim 1, wherein the compound has the following structure (IA):

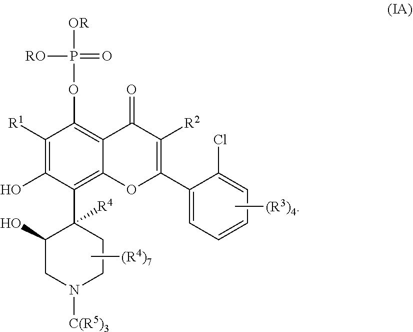

5. The compound of claim 1, wherein the compound has the following structure (IB):

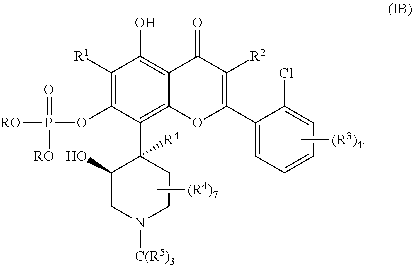

6. The compound of claim 1, wherein the compound has the following structure (IC):

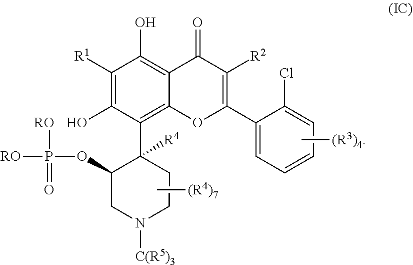

7.-27. (canceled)

28. The compound of claim 1 having one of the following structures: 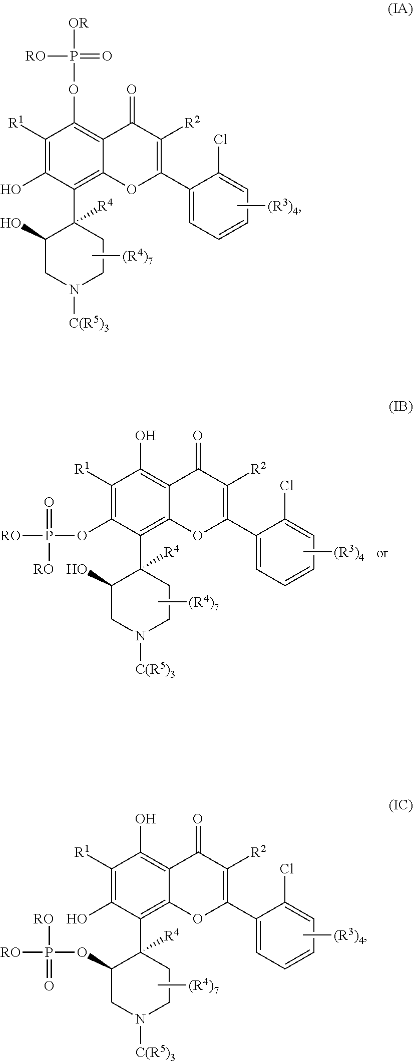
or a pharmaceutically acceptable salt thereof, wherein each R is H; R³ is the same at each occurrence; R⁴ is the same at each occurrence; R⁵ is the same at each occurrence; and the values for R¹, R², R³, R⁴ and R⁵ are as listed in the table below:

| R¹ | R² | R³ | R⁴ | R⁵ |
|---|---|---|---|---|
| D | D | H | H | H |
| D | D | D | H | H |
| D | D | D | D | H |
| D | D | D | D | D |
| H | D | D | H | H |
| H | D | D | D | H |
| H | D | D | D | D |
| H | H | D | H | H |
| H | H | D | D | H |
| H | H | D | D | D |
| D | H | D | D | D |
| H | H | H | D | H |
| H | H | H | D | D |
| D | H | H | D | D |
| D | D | H | D | D |
| D | D | H | H | D |
| D | D | D | H | D |

29. A pharmaceutically acceptable salt of a compound according to claim 1.

30.-33. (canceled)

34. A pharmaceutical composition comprising a pharmaceutically acceptable carrier or excipient, and a compound having the following structure (I): 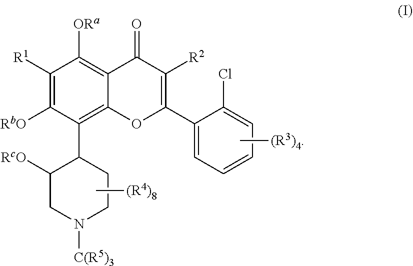
or a stereoisomer, tautomer or pharmaceutically acceptable salt thereof, wherein:
R^{a}, R^{b} and R^{c} are each independently H, D or —P(═O)(OR)₂,
R, R¹, R², R³, R⁴ and R⁵ are, at each occurrence, independently H or D,
wherein at least one occurrence of R, R^{a}, R^{b}, R^{c}, R¹, R², R³, R⁴ and R⁵ is D.

35. (canceled)

36. A method for treating a disease associated with overexpression of a cyclin-dependent kinase (CDK) in a mammal in need thereof, the method comprising administering a therapeutically effective amount of a compound to the mammal, the compound having the following structure (I): 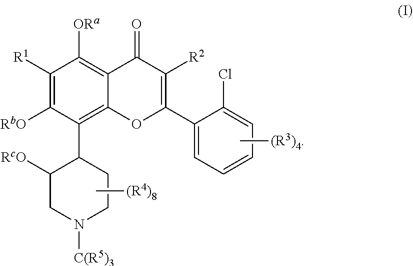
or a stereoisomer, tautomer or pharmaceutically acceptable salt thereof, wherein:
R^{a}, R^{b} and R^{c} are each independently H, D or —P(═O)(OR)₂;
R, R¹, R², R³, R⁴ and R⁵ are, at each occurrence, independently H or D;
wherein at least one occurrence of R, R^{a}, R^{b}, R^{c}, R¹, R², R³, R⁴ and R⁵ is D.

37. The method of claim 36, wherein the disease is cancer.

38. The method of claim 37, wherein the cancer is a hematologic cancer.

39. The method of claim 38, wherein the hematologic cancer is selected from acute myelogenous leukemia (AML), multiple myeloma, follicular lymphoma, acute lymphoblastic leukemia (ALL), chronic lymphocytic leukemia (CLL) and non-Hodgkin's lymphoma.

40-43. (canceled)

44. The method of claim 36, wherein the method comprises orally administering a therapeutically effective amount of a compound of claim 1, or a stereoisomer, tautomer or pharmaceutically acceptable salt thereof to the mammal.

45. The method of claim 36, further comprising administering an additional therapeutic agent to the mammal.

46. (canceled)

47. (canceled)

48. The method of claim 36, wherein the mammal has an MCL-1 dependency percentage of at least 15%, the MCL-1 dependency percentage having been obtained by an in vitro method comprising contacting a first portion of a plurality of cancer cells with a profiling peptide comprising a cellular uptake moiety and an MCL-1 binding domain, the MCL-1 binding domain having the sequence of SEQ ID NO:1 with 0-8 modifications.

49.-54. (canceled)

55. The method of claim 48, wherein the method further comprises administering a therapeutically effective amount of a DNA methyltransferase inhibitor.

56. The method of claim 55, wherein the DNA methyltransferase inhibitor is azacitidine or decitabine.

57. (canceled)

58. The method of claim 37, wherein the cancer comprises a solid tumor.
